SEQUENCE LISTING (938 sequences)

SEQ ID NO: 1 - DNA, 2390 bp, Cannabis sativa
acccatgcatgcatgcatgcatgcatgcatatagatatgtattatcgttttctgcatgcagtttctctttgagcgttgaagaaaagagcagccacaggaggaccaaggtcattttgggtagcaaagaggcgcgtgttgaaacgctctctcgatgaaggtggcattaccactgtttgtcttctcttctgtttaaacattaagaacacaaacctatgtattcctatgtttggtcgtggcatttcatagctcactatctcttctcctacatatattaattcatttaaacattaattattctcataattgattaattcattcttacaaaattaaaaacatatacatatttactatataataattaccaaatgacacatcagttgttcctggaatgtctgtcactatcctgcaattatttcaacaacaaatattaatttcaactcaaattaatttagaaactcataccattttaaattttgccttttgcttaagtttgattaaaatctctattttgttaaattataaattggattatgcatttttcaaagtataaaatatagtatatatatattctgattggctgatttttgacaagaaaaacgcaataataatttaatgtataggtgttacaaatgattacattttctgtatttatttgtgttaaaaaatcttcttcaagttggttatatgaaaaatttgtcaaaaattgaattcaaaatgctaaagtgtactatttagaaaatacaagatgtaatttattatttaacaaaataaaaattttataaaatataaagtccaactattataaccctagataatatatgtgtatacattacatattagagaaaaaatatattactaattaaattctagaaagtaatatattattgagaaagaacatgttttcatttcattcaaaagtaaaataatagggtaattaggatttttgtcccttaaaattttgatattactaaattatgctctctgaactttaagcttgttaaaaatgtctcttgaactattgagattgttaaatttaaggactttgtttaatttcattcaattttactatttcagtgattgtttatgtactaaattatgctccctaaattttaatatctaccaaatcatgcctctgaactttcctccatgttaattagtttttttactaaaattggacaaaaattcttaaatccaacaatctcaatagtttagagaacatcatttagtaaaattgatatatataatataatatatatatattggacagctgataactaattgttgtatattattattaatttaatattatttgctattatgactttaaagtgatatatataattaatcaaaaacttttttaattatttgttgaaattctgagatcttgtgatgactaattaatgatcatgtgtggtctcaccagttgcatatattcccttaagtatacttattattatactattcccctatatatatatattatatataaaaaaaaagaagaatattaataattatatatacatagattagacatattataatattaaattaatgaatattaagaccttcccccctaacgatcgatcaagatgtgtacagtgtctcaaattatacgacagcttaattagatgaaaaatatttatatttatattgaaaaaagtgaaaatagctatacacaagttgaataatattatatatatttatataagataagaattatataagaaatatgtatgatcggaattttattggataaaaaaaaattatcaatattatatatagtattaggtatatatttaaatttttagtttatatctttcaatctctctctctagtaataaaataacttatatatgaatattataggtaattaattgtctaataatatatttaataataagtgatataacccaaaaaacttttgtcatcacacattatattttaatatctatagtaatttgtagcaatgtatatatgtagtaatttgttcttaaatataatttaattattttgtagttaccagtgtaaatgctccctcaaataaggatcactagggccaggaacatcaggatcagtcatcacctaattaatattatttacaacaaaataaacaacattatatttaacaaaatgatgttatatataattaatttaagacatgtaattaattaattattaccagtgtgaaaaaggatctcatgtcaccaccatgaacctcaacttttggcttgatagtaattgtggaaggaaacaactcatgaccattgtagactttcttgtttgagttgtaagtcactgtaaattttagggttggagagaaaacatcaaccacatctcctatcactcttccaataataagaggattatctgacattcttgccattaatttt

SEQ ID NO: 2 - DNA, 531 bp, Cannabis sativa
  CDS (1)..(531): CsSP-1 coding sequence
atggcaagaatgtcagataatcctcttattattggaagagtgataggagatgtggttgatgttttctctccaaccctaaaatttacagtgacttacaactcaaacaagaaagtctacaatggtcatgagttgtttccttccacaattactatcaagccaaaagttgaggttcatggtggtgacatgagatcctttttcacactggtaatgactgatcctgatgttcctggccctagtgatccttatttgagggagcatttacactggatagtgacagacattccaggaacaactgatgtgtcatttggtggagaagagatagtgagctatgaaatgccacgaccaaacataggaatacataggtttgtgttcttaatgtttaaacagaagagaagacaaacagtggtaatgccaccttcatcgagagagcgtttcaacacgcgcctctttgctacccaaaatgaccttggtcctcctgtggctgctcttttcttcaacgctcaaagagaaactgcatgcagaaaacgataa

SEQ ID NO: 3 - protein, 176 aa, Cannabis sativa
MARMSDNPLIIGRVIGDVVDVFSPTLKFTVTYNSNKKVYNGHELFPSTITIKPKVEVHGGDMRSFFTLVMTDPDVPGPSDPYLREHLHWIVTDIPGTTDVSFGGEEIVSYEMPRPNIGIHRFVFLMFKQKRRQTVVMPPSSRERFNTRLFATQNDLGPPVAALFFNAQRETACRKR

SEQ ID NO: 4 - DNA, 1195 bp, Cannabis sativa
tatatgtcattattggcagggcatatagttaattaattgcttcttcttgatctagcagcagtttctctttgtgcattgaagtatacaatagccacggggagacccagattgttgtcttcagaaaatgtccttgtgttaaaacagtctctcaaagctggtgctctcacagtttgtcttcctctttgcttgaataagatgaatacaaatctatgaatccccaccaccggttttggagcttcataactcacaatttcttttcctgttttcgtttataaatatatatatatatatatgtatacacgtacaaaaagctccatttagatatttatttcgttatatatattagtcttatatttatctaaaagaccacactaccaaaagaaaaaaacataatgcatggttcattgtgtcacacacacatatatacgatagtgaggacatgaaatactcaccaaaggatacattagttgtacctggaatgtctgtgactatcctggaaagaatacacgtattataattaaatatccaatattaatggaaaggatcacatatatatatatatataaatatacaagaagaggttacaaaaaaaaaaagtaaaaatatataataagtattatttctttaaactaattatattacaaaccagtgaaggtgttcttttaagtagggatcactagggcttggagcatctggatctaccataatcttcaagaaataagtggaaaaaagggtaaaataaatatatgttgcttctagagacagtataaaagttagagatatatctctatatatatatatatgatattttaatgattaaacttacgagtgtgtaagattctcatatcttctccaccaatatctactcgaggtttagacaaaatattagaaggcatgaactcgtagccattacaaacttgcttatcagggttgttatatatgacagtcatttttacactaggattgaaattgtcaaccacatctcctattactcttgccacacttaaagtttgtattgctgaccttgacattattattatataaatataataatagatcagatatatgataaggatgagaggaaagagctaactatatatagtagtttaaaatttaactcaattgtagtagtagtagtagagatcgagttaggtgatcaatgggtatttataagccagctagggcaagttggaat

SEQ ID NO: 5 - DNA, 516 bp, Cannabis sativa
  CDS (1)..(516): CsSP-2 CDS
atgtcaaggtcagcaatacaaactttaagtgtggcaagagtaataggagatgtggttgacaatttcaatcctagtgtaaaaatgactgtcatatataacaaccctgataagcaagtttgtaatggctacgagttcatgccttctaatattttgtctaaacctcgagtagatattggtggagaagatatgagaatcttacacactcgtaagtttaatcatatggtagatccagatgctccaagccctagtgatccctacttaaaagaacaccttcactggtttgtaataggaaaagaaattgtgagttatgaagctccaaaaccggtggtggggattcatagatttgtattcatcttattcaagcaaagaggaagacaaactgtgagagcaccagctttgagagactgttttaacacaaggacattttctgaagacaacaatctgggtctccccgtggctattgtatacttcaatgcacaaagagaaactgctgctagatcaagaagaagcaattaa

SEQ ID NO: 6 - protein, 171 aa, Cannabis sativa
MSRSAIQTLSVARVIGDVVDNFNPSVKMTVIYNNPDKQVCNGYEFMPSNILSKPRVDIGGEDMRILHTRKFNHMVDPDAPSPSDPYLKEHLHWFVIGKEIVSYEAPKPVVGIHRFVFILFKQRGRQTVRAPALRDCFNTRTFSEDNNLGLPVAIVYFNAQRETAARSRRSN

SEQ ID NO: 7 - DNA, 956 bp, Cannabis sativa
tactgagaatatatatatatattagtggtaggtaggtagagctagcgtcttctagctgcggtttctctctgagcgttgaaataaacggcagcgacaggaagaccgaggtcgttttgagcagcgaaactttttgtgttgaaatgatcctttgagcttggtatactcaccgagtctcggcatttctgtttgaacagaacaaatacgaacctgtgaatccctatatttggctttgggctctcatagctaaccacttcccttcctgtgtacatatccattttaattttatatttataaaaataatattatatacatatataattaataatggatttaattaataatattaccaaatgtagcgtctgtggtgcctggtatgtctgtcacaatcctgtccatgtttcaaagaagaaaataattattacaatcaggatatcttaggtctatatatagaaaaacatgcatacatatattactgtctcctaaaatagtttgttaattaaaccaaattataatcttactgacatttggattatatacacataataacaactatttattataccagtgaaggtgctcccttaagtaaggatcacttgggccaggaacatctggatctgtcatgacctaaaataaaataaagagagagagaaaaaaaaattaccaaaaaaagaacaacattaattaaaacatttaaacaaaaccttgttcatcatttagagatctaacaaaacaattatcataatatatatttttttattagtgtgaagaaggatctcatatcagctccttgaatctcaaccctaggtttggagatgacaactgaaggaaagagctcatgtccattacagacttgcttcttgttataagagacagacatttttatggatggactgaaagactctagaacatctcctatcactcttccaaccacaagaggctctaacattcttgccata

SEQ ID NO: 8 - DNA, 519 bp, Cannabis sativa
  CDS (1)..(519): CsSP-3 CDS
atggcaagaatgttagagcctcttgtggttggaagagtgataggagatgttctagagtctttcagtccatccataaaaatgtctgtctcttataacaagaagcaagtctgtaatggacatgagctctttccttcagttgtcatctccaaacctagggttgagattcaaggagctgatatgagatccttcttcacactaataatgacagatccagatgttcctggcccaagtgatccttacttaagggagcaccttcactggattgtgacagacataccaggcaccacagacgctacatttggaagggaagtggttagctatgagagcccaaagccaaatatagggattcacaggttcgtatttgttctgttcaaacagaaatgccgagactcggtgagtataccaagctcaaaggatcatttcaacacaaaaagtttcgctgctcaaaacgacctcggtcttcctgtcgctgccgtttatttcaacgctcagagagaaaccgcagctagaagacgctag

SEQ ID NO: 9 - protein, 172 aa, Cannabis sativa
MARMLEPLVVGRVIGDVLESFSPSIKMSVSYNKKQVCNGHELFPSVVISKPRVEIQGADMRSFFTLIMTDPDVPGPSDPYLREHLHWIVTDIPGTTDATFGREVVSYESPKPNIGIHRFVFVLFKQKCRDSVSIPSSKDHFNTKSFAAQNDLGLPVAAVYFNAQRETAARRR

SEQ ID NO: 10 - DNA, 3107 bp, Cannabis sativa
acgaaaaacttggattcttctctctatattaatatataacaagtttggagtagtagagtatagcaatatagtgctgttgttgttctttttttatttaattaataataataataattaattaagaatatatggctagagatagggaccctcttgtggttggtagagtaataggagatgttttggatccttttacaaggtcagtctctcttagagtgagttatggtaatagagaggtcaacaatggttatgagcttaaaccttcccaaattgttaaccaacctcgtgttgatattggtggtgatgacctaaggaccttctacactttggtaaattaattaattaattaattacctcaatattcataatgatttcatctataaatatatatatatataaatatgtatattatgatcaatgtcatatattgaaaattattacaggtcatggtggatccggatgctcctagccctagcaaccccaatctaagggagtatttgcattggtaattaattaattaactaattaattaattgattatatattattattaaatagatgagggtttcatatcatgataagtagtataaaataattaattaaaattaataactttttttgagtaaatgatgatgagaaattattattattactcaaaagtatatgcttatatttttaattttgtataaaactttggaacgaatacaaaataaattatatattatatatgtttaaattatgtttttaattagtacaattaactatttattcttttttttagagaacactttttctaaattatttccaacatgtgttgtgtgcatgaaattgtttaattatgacacttatttgctaattcactacaccaaaatatgtctttactttttggtcattaattatgcattttataaagatgttagttgggcgtctgttgaaaaaatattgacgctaatattagttactgatatgttgagtataaagtgaaacgatgtgttttcgtgtaaatatataatgtgtcagttttgaaatcgataatgtcaataattgacgcttagactcgatagtgtcgattatataattaataaagtacttttagtgtcagttttgaattggaacttaccctaataattacagaattttgtaaaactataaccctaaagcttgttttaatttgttaaagtgactaggttattataatcatatttctagctaatatattaagtacccttaaattctttttacatgtttattaagccacaaattccatatatgctagctatataaatataaatatggaaaggatatatgggaatatacaatatatatgaatatataaaaagctgatgagaaagaatgtggtttggggatgaattttgaatgatgaagggatgaggctgtgtgtgtggaatcagttttaccctgctagctatagcatgtccccttttgtcccaccattaaaatggccacctcacactaagaaggttttcacaaattaatgtgtcatactctaagatcaacacatgtcaatatatatacatacacatgtatagggcattattagtaatttttatacattacttcagtaaattacacatatttcaactttgacccgaatttgaattcgaacatagactcagatttggaccctaaattcgaccctgaaccctgaaacacgaacttagactcagacctggacctggaccttgacccaaaccccgacccgaaccccgactcgaaccccgaaactagactggacctggactctggcctaggacctagctcgggtctagagttcaggtccagtccggggtccgaggtcaggatctgggttcgtgttctggggttcaggttgggttgggtctggagtctgggtctagggtccagattcaggatcgggttcaggttaaggtttgagtctgagtccaggtataggttctggtcgggatctgagttcgagagtttgaattcaaggtccaatttggagttggatgagttcatgtcatggtaaaatattatttttttagttaaaaaaaaattaaaataatcttaaaaaactgagccaaacactattaaaattttaaaataataaaaaataattttcaattctcacatttaaaacacaattttaaaaacaaaaaatgaaaaacacagtcaaatactcattattcataatttttaaattagagggaatgccctataagtaagataaaaataatatttcttatcataattttgtcttatatatactttttaactattttaatattaaattattagtttatgtagttatattcgttgtagttaccgtgaatattcggtacagtaaattaaggatggtaaaatagtgttctaaatttgttattatgattagataatagtactactttttcaatgaatttgttattaccacaatttatatttttatatatattattaatctagataatggtaatctaagagtcatttttgaactaataattgagctttgacctataatatatagtataattataataattatacatgtttggcaatttcaatttaggttggtgactgatattcctggaactaccgggccaagcttcggtgagccgttcatacttcataataatactcttatttactattttttctattatattaaattaagaacgctgtaaacaaaatagtgcggattaataattttttttaaaataatattaaataattataatatataatgttgtattacaaagtatataatttgttttttaattaattgtttattatgtgtaggacaagaggtggtgtgctacgagaacccgcggccgacggtggggattcatcggtatgtatttgtgttgttccggcaattgggaaggcaaacggtgttcgcgccggggtggcgtcagaacttcaacaccaaagactttgctgagctttataaccttggtttgcctgttgctgccctttattacaactgccaaagggagacaggatctgggggaaggaggtcctccacatagacatatatatatatatatatatcatcatcatatatcatatagatactaatatatattatatgatgattcttaatgatgatatcatggttttattatcaatgccat

SEQ ID NO: 11 - DNA, 492 bp, Cannabis sativa
  CDS (1)..(492): CsSP5G-1 CDS
atggctagagatagggaccctcttgtggttggtagagtaataggagatgttttggatccttttacaaggtcagtctctcttagagtgagttatggtaatagagaggtcaacaatggttatgagcttaaaccttcccaaattgttaaccaacctcgtgttgatattggtggtgatgacctaaggaccttctacactttggtaatggtggatccggatgctcctagccctagcaaccccaatctaagggagtatttgcattggggacaagaggtggtgtgctacgagaacccgcggccgacggtggggattcatcggtatgtatttgtgttgttccggcaattgggaaggcaaacggtgttcgcgccggggtggcgtcagaacttcaacaccaaagactttgctgagctttataaccttggtttgcctgttgctgccctttattacaactgccaaagggagacaggatctgggggaaggaggtcctccacatag

SEQ ID NO: 12 - protein, 163 aa, Cannabis sativa
MARDRDPLVVGRVIGDVLDPFTRSVSLRVSYGNREVNNGYELKPSQIVNQPRVDIGGDDLRTFYTLVMVDPDAPSPSNPNLREYLHWGQEVVCYENPRPTVGIHRYVFVLFRQLGRQTVFAPGWRQNFNTKDFAELYNLGLPVAALYYNCQRETGSGGRRSST

SEQ ID NO: 13 - DNA, 2867 bp, Cannabis sativa
taataatataattaattaagaatatatggctagagatagggaccctcttgtggttggtagagtaataggagatgttttggatccttttacaaggtcagtctctcttagagtgagttatggtaatgagaggtcaacaatggttatgagcttaaaccttcccaaattgttaaccaacctcgtgttgatattggtggtgatgacctaaggaccttctacactttggtaaattaattaattaattaattacctcaatattcataatgatttcatctataaatatatatatatataatatgtatattatgatcaatgtcatatattgaaattattacaggtcatggtggatccggatgctcctagccctagcaaccccaatctaagggagtatttgcattggtaattaattaattaactaattaattaattgattatatattattattataatagatgagggtttcatatcatgataagtagtataaaataattaattaaaattaataactttttttgagtaatgatgatgagaaattattattatattactcaaaagtatatgcttatattttaattttgtataaaaatttggaacgaatacaaaataaattatatattatatagtttaaattatgtttttaattagtacaattaactatttattcttttttttagagaacactttttctaattatttccaacatgtgttgtgtgcatgaaattgtttaattatgacacttatttgctaattcactacaccaaaatatgtctttatcttttggtcattaattatgcattttataaagatgttagttgggcgtctgttgaaaaaatattgacgctaatattagttactgatatgtgagtataagtgaaacgatgtgttttcgtgtaaatatataatgttcagttttgaaatcgataatgtcaataattgacgcttagactcgatagtgtcgattatataattaataaagtacttttagtgtcagttttgaattggaacttaccctaataattacagaattttgtaaaactataaccctaaagcttgttttaatttgttaaagtgactaggttattataatcatatttctagctaatatattaagtacccttaaattcttttacatgtttataagccacaatccatatatgctagctatataaatataaatatggaaaggatatatgggaatatacaatatatatgaatatataaaagctgatgagaaagaatgtggtttggggatgaattttgaatgatgaagggatgaggctgtgtgtgtggaatcagtttaccctgctagctatagcatgtccccttttgtcccccattaaaatggccacctcacactaagaaggttttcacaaattaatgttgtcatactctaatcaacacatgtcaatatatatacatacacatgtatagggcattattagtaatttttatacattacttcagtaaattcacatatttcaactttgacccgaatttgaattcgaacatagactcagatttggaccctaattcgaccctgaaccctgaaacacgaacttagactcagacctggactggaccttgacccaaaccccgacccgaaccccgactcgaaccccgaaactagactggacctggactctggcctaggacctagctcgggtctaggtcaggcgtccgggtccgaggtcagatctgggttcgtgttctggggttcaggttgggttgggtctggagtctgggtctagggtccagattcaggatcgggttcaggttaaggtttgagtctgagtccaggtataggttctggtcgggatctgagttcagagtttgaattcaaggtccaatttggagttggatgagttcatgtcatggtaaaatattatttttttagttaaaaaaaaattaaaataatcttaaaaaactgagccaaacactattaaaattttaaaataataaaaataattttcaattctcacatttaaaaacacaattttaaaaacaaaaaatgaaaaacacagtcaaatactcattattcataatttttaaaattagagggaatgccctataagtaaagataaaaataatatttcttatcataattttgtcttatatatactttttaactattttaatattaaattattagtttatgtagttatattcgttgtagttaccgtgaatattcggtacagtaaattaaggatggtaaaatagtgttctaaatttgttattatgattagataatagtactactttttcaatgaatttgttattaccacaatttatatttttatatattattaatctagataatggtaatctaagagtcatttttgaactaataattgagctttgacctatatattagtataattataataattatacatgtttggcaatttcaattttaggttggtgactgatattcctggaactaccgggccaagcttcggtgagccgttcatacttcataataatactcttatttactattttttctattatattaaattaagaacgctgtaaacaaaatagtgcggataataattttttttaaaataatattaaatatataatatatgttgttacaaagtatataatttgttttttaattaattgtttattatgtgtaggacaagaggtggtgtgctacgagaacccgcggccgggtggggattcatcggtatgtatttgtgttgttccggcaattgggaaggcaaacggtgttcgcgccggggtggcgtcagaacttcaacaccaaagactttgctgagctttataaccttggtttgcctgttgctgccctttattacaactgccaaagggagacaggatctgggggaaggaggtcctccacatagacata

SEQ ID NO: 14 - DNA, 531 bp, Cannabis sativa
  CDS (1)..(531): CsSP5G-2 CDS
atggctagagatagggaccctcttgtggttggtagagtaataggagatgttttggatccttttacaaggtcagtctctcttagagtgagttatggtaatgagaggtcaacagatggttatgagcttaaaccttcccaaattgttaaccaacctcgtgttgatattggtggtgatgacctaaggaccttctacactttggtaaatatggtggatccggatgctcctagccctagcaaccccaatctaagggagtatttgcattggaggttgggttgggtctggagtctgggtctagggtccagattcaggatcgggttcaggttaaggtttcgcggccgggtggggattcatcggtatgtatttgtgttgttccggcaattgggaaggcaaacggtgttcgcgccggggtggcgtcagaacttcaacaccaaagactttgctgagctttataaccttggtttgcctgttgctgccctttattacaactgccaaagggagacaggatctgggggaaggaggtcctccacatag

SEQ ID NO: 15 - protein, 176 aa, Cannabis sativa
MARDRDPLVVGRVIGDVLDPFTRSVSLRVSYGNERSTDGYELKPSQIVNQPRVDIGGDDLRTFYTLVNMVDPDAPSPSNPNLREYLHWRLGWVWSLGLGSRFRIGFRLRFRGRVGIHRYVFVLFRQLGRQTVFAPGWRQNFNTKDFAELYNLGLPVAALYYNCQRETGSGGRRSST

SEQ ID NO: 16 - DNA, 1673 bp, Cannabis sativa
attatgattataattatatataattatatttatgaagttgatgacctccttccaccacagccaagttccttttggcagttgaagtaaacgacagcaacaggcaatccaaggttgtagatctcagcaaaatctcttgtgttgaagttatgtcgccacccaggtgcatacactgtctgccttcctaactgtcgaaacagaacaaacacatagcgatgtattcccaccgtcggtctagggctctcgtagcacatcacttcttgtcctacatatatgcgtatatacacacatcattaaatcatcacacataatatatatatatctttatatatcaaaagtaattatcgaatgataattcttgattaaaattttttttaacttcttaaaataacattgttaacttatcgttaacttattggtgttattagttctgtaaaatatatataattgtgttttgtgcataaatattaacaaaaaaatgagtgtattaatatgtaccaaaggctgttcccgtagttgcgggaatatcagtcaccaacctgaaaaagttatacacttaaattaaatttaaacttaagaaaaataaaattaataattattagtggcacacactattgatggtgccttgtcattcgttagtaatattctctaaaaattatcaaattaaattataccgaaatgatattactaagtacataccaatgcaaatattctttaagattaggttcgctagggttaggagcatcagatcaaccataacctgcaacattaacagtattaattatacgttaatttataaatgatttattatgaagatagagttaagatatatatatatatatactagttagatattacgtgcgatgcacgtatacttaattttatgctaggtgtttttttactttatttacaaacaatacaataaataattaaaaaaaaaaaaaacatattatttagagaagaataatagagaaaaatagttaacgatgattcgattacagaatttgtgtatttgtgcaattataagttaggccttattgttgtgggtctttgacatgtgattagtatcaaatatactaattaacagtgttaattttaaacggttaaacagaaaattcacaacaaatctgttaaacagaacaaatatcagttcaaatattaatgtgattcgttttatttttattttattgtatataaataatattttattattttattaaatataaaaaaaagtcacccactaatttttcataaaccaagaatttagttatataggcacactttatatagaatagatatagatataaatactgaccaaagtgtagaaagtcctgagatcatctccaccaatctcaaccctaggtgagttagcaacttgagaaggtctaaggtcagtaccattgttaatagccctaatcccactatatgatactttaagagagacagtttttgtgaaagggtccaacacatcacttatcactctcccaaccacaagaggatccctattagccatttatatatataacaactatatacagtataaatatgtagagagatagagagaagaagagggtttgatgagtttttggttgtataatttggtggctgacattcaacaatttatggctctatttatagttatcacttatattctctctgttagcttttgtaacatc

SEQ ID NO: 17 - DNA, 537 bp, Cannabis sativa
  CDS (1)..(537): CsSP5G-3 CDS
atggctaatagggatcctcttgtggttgggagagtgataagtgatgtgttggaccctttcacaaaaactgtctctcttaaagtatcatatagtgggattagggctattaacaatggtactgaccttagaccttctcaagttgctaactcacctagggttgagattggtggagatgatctcaggactttctacactttggtccaggttatggttgatctgatgctcctaaccctagcgaacctaatcttaaagaatatttgcattggtatgtacttagtaatatcatttcggtataatttaattggacaagaagtgatgtgctacgagagccctagaccgacggtgggaatacatcgctatgtgtttgttctgtttcgacagttaggaaggcagacagtgtatgcacctgggtggcgacataacttcaacacaagagattttgctgagatctacaaccttggattgcctgttgctgtcgtttacttcaactgccaaaaggaacttggctgtggtggaaggaggtcatcaacttcataa

SEQ ID NO: 18 - protein, 178 aa, Cannabis sativa
MANRDPLVVGRVISDVLDPFTKTVSLKVSYSGIRAINNGTDLRPSQVANSPRVEIGGDDLRTFYTLVQVMVDLMLLTLANLILKNICIGMYLVISFRYNLIGQEVMCYESPRPTVGIHRYVFVLFRQLGRQTVYAPGWRHNFNTRDFAEIYNLGLPVAVVYFNCQKELGCGGRRSSTS

SEQ ID NO: 19 - DNA, 1434 bp, Cannabis sativa
aaattggtccacacaactcccaataactttacagtagtcggccattgctataagaattatataaataaataaatgtttttactaattaagagaatccacaatatatattagaaccttttgatgttacaaaagctaaaagagagaatataagttataactataaatagagccataaattgttgaatgtcagccaccaaattatataaccaaaactcatcaaaccctcttctgatctctctctacatatatttatatatagttgttatatatataaatggctaatagggatcctcttgtggttgggagagtgataagtgatgtgttggaccctttcacaaaaactgtgtctcttaaagtatcatatagtgggaatagggctattaacaatggtactgaccttagaccttctcaagttgctaactcacctagggttgagattggtggagatgatctcaggactttctacactttggtatatatatatatcttcactactctatcttcataataaatcattgataaattaatgtataattaatactgttaatgttgcaggttatggttgatcctgatgctcctaaccctagcgaacctaatcttaaagaatatttgcattggtatgtacttagtaatatatcacttcgagtatcatataatttaatttgataacttttagatatattacttaggaatcacaaggcaccatcaataagtgtgtgccactaataattattaatttttatttttcttaatttagtttaaatttaattaaagtgtataattttgtcaggttggtgactgatattcccgcaactacgggaacaggctttggtacatatttatacactcattttttttgttaatatttatgcacaaaacacaattataatttattttacagaaaataacaccaataagttaatggtaagttaacaacgttatttaaaaaaaatgttaaattaagaattatcattaaataattacttttaatatataaagatatagtgtgtgtgatgatttaatgatgggcgtacgcatatatgtaggacaagaagtgatgtgctacgagagcccaagaccgacggtgggaattcatcgctttgtgtttgttctgtttcgacagttaggaaggcagacagtgtatgcacccgggtggcgacataacttcaacacaagagattttgctgaaatctacaaccttggattgcctgttgctgccgtttacttcaactgccaaaaggaacttggctgtggtggaaggaggtcatcaacatcataaatattaattatataatatactaatcatattaatcataatctatccatctatctatctatatatgtcatattattatatattgaataatgcttgagacacaacaactacaactattaattttatatattctataagttattgatatatctatatat

SEQ ID NO: 20 - DNA, 531 bp, Cannabis sativa
  CDS (1)..(531): CsSP5G-4 CDS
atggctaatagggatcctcttgtggttgggagagtgataagtgatgtgttggaccctttcacaaaaactgtgtctcttaaagtatcatatagtgggaatagggctattaacaatggtactgaccttagaccttctcaagttgctaactcacctagggttgagattggtggagatgatctcaggactttctacactttggtaatggttgatcctgatgctcctaaccctagcgaacctaatcttaaagaatatttgcatagtttaaatttaattaaagtgtataattttgtcaggttgggacaagaagtgatgtgctacgagagcccaagaccgacggtgggaattcatcgctttgtgtttgttctgtttcgacagttaggaaggcagacagtgtatgcacccgggtggcgacataacttcaacacaagagattttgctgaaatctacaaccttggattgcctgttgctgccgtttacttcaactgccaaaaggaacttggctgtggtggaaggaggtcatcaacatcataa

SEQ ID NO: 21 - protein, 176 aa, Cannabis sativa
MANRDPLVVGRVISDVLDPFTKTVSLKVSYSGNRAINNGTDLRPSQVANSPRVEIGGDDLRTFYTLVMVDPDAPNPSEPNLKEYLHSLNLIKVYNFVRLGQEVMCYESPRPTVGIHRFVFVLFRQLGRQTVYAPGWRHNFNTRDFAEIYNLGLPVAAVYFNCQKELGCGGRRSSTS

SEQ ID NO: 22 - DNA, 20 bp, Artificial Sequence
  Synthetic polynucleotide
tatatatattaagactacgt

SEQ ID NO: 23 - DNA, 20 bp, Artificial Sequence
  Synthetic polynucleotide
ataagtgtgtaagaggctcg

SEQ ID NO: 24 - DNA, 20 bp, Artificial Sequence
  Synthetic polynucleotide
ttattatataagtgtgtaag

SEQ ID NO: 25 - DNA, 20 bp, Artificial Sequence
  Synthetic polynucleotide
gcatgcatgcatgcatgcat

SEQ ID NO: 26 - DNA, 20 bp, Artificial Sequence
  Synthetic polynucleotide
tgcatgcatgcatgcatgca

SEQ ID NO: 27 - DNA, 20 bp, Artificial Sequence
  Synthetic polynucleotide
ttgaagaaaagagcagccac

SEQ ID NO: 28 - DNA, 20 bp, Artificial Sequence
  Synthetic polynucleotide
aagaaaagagcagccacagg

SEQ ID NO: 29 - DNA, 20 bp, Artificial Sequence
  Synthetic polynucleotide
aaatgaccttggtcctcctg

SEQ ID NO: 30 - DNA, 20 bp, Artificial Sequence
  Synthetic polynucleotide
gagcagccacaggaggacca

SEQ ID NO: 31 - DNA, 20 bp, Artificial Sequence
  Synthetic polynucleotide
tttgctacccaaaatgacct

SEQ ID NO: 32 - DNA, 20 bp, Artificial Sequence
  Synthetic polynucleotide
caggaggaccaaggtcattt

SEQ ID NO: 33 - DNA, 20 bp, Artificial Sequence
  Synthetic polynucleotide
aggaggaccaaggtcatttt

SEQ ID NO: 34 - DNA, 20 bp, Artificial Sequence
  Synthetic polynucleotide
ggtcattttgggtagcaaag

SEQ ID NO: 35 - DNA, 20 bp, Artificial Sequence
  Synthetic polynucleotide
ttgaaacgctctctcgatga

SEQ ID NO: 36 - DNA, 20 bp, Artificial Sequence
  Synthetic polynucleotide
aaacgctctctcgatgaagg

SEQ ID NO: 37 - DNA, 20 bp, Artificial Sequence
  Synthetic polynucleotide
acagaagagaagacaaacag

SEQ ID NO: 38 - DNA, 20 bp, Artificial Sequence
  Synthetic polynucleotide
gaccaaacataggaatacat

SEQ ID NO: 39 - DNA, 20 bp, Artificial Sequence
  Synthetic polynucleotide
aacctatgtattcctatgtt

SEQ ID NO: 40 - DNA, 20 bp, Artificial Sequence
  Synthetic polynucleotide
gaaatgccacgaccaaacat

SEQ ID NO: 41 - DNA, 20 bp, Artificial Sequence
  Synthetic polynucleotide
tgtattcctatgtttggtcg

SEQ ID NO: 42 - DNA, 20 bp, Artificial Sequence
  Synthetic polynucleotide
ttaaatgaattaatatatgt

SEQ ID NO: 43 - DNA, 20 bp, Artificial Sequence
  Synthetic polynucleotide
ggaacaactgatgtgtcatt

SEQ ID NO: 44 - DNA, 20 bp, Artificial Sequence
  Synthetic polynucleotide
aatgacacatcagttgttcc

SEQ ID NO: 45 - DNA, 20 bp, Artificial Sequence
  Synthetic polynucleotide
aggatagtgacagacattcc

SEQ ID NO: 46 - DNA, 20 bp, Artificial Sequence
  Synthetic polynucleotide
tttgttgttgaaataattgc

SEQ ID NO: 47 - DNA, 20 bp, Artificial Sequence
  Synthetic polynucleotide
gcaaaaggcaaaatttaaaa

SEQ ID NO: 48 - DNA, 20 bp, Artificial Sequence
  Synthetic polynucleotide
tttaatcaaacttaagcaaa

SEQ ID NO: 49 - DNA, 20 bp, Artificial Sequence
  Synthetic polynucleotide
tattttgttaaattataaat

SEQ ID NO: 50 - DNA, 20 bp, Artificial Sequence
  Synthetic polynucleotide
tagtatatatatattctgat

SEQ ID NO: 51 - DNA, 20 bp, Artificial Sequence
  Synthetic polynucleotide
gcaataataatttaatgtat

SEQ ID NO: 52 - DNA, 20 bp, Artificial Sequence
  Synthetic polynucleotide
ttaaaaaatcttcttcaagt

SEQ ID NO: 53 - DNA, 20 bp, Artificial Sequence
  Synthetic polynucleotide
ttatctagggttataatagt

SEQ ID NO: 54 - DNA, 20 bp, Artificial Sequence
  Synthetic polynucleotide
gtatacacatatattatcta

SEQ ID NO: 55 - DNA, 20 bp, Artificial Sequence
  Synthetic polynucleotide
tgtatacacatatattatct

SEQ ID NO: 56 - DNA, 20 bp, Artificial Sequence
  Synthetic polynucleotide
tcattcaaaagtaaaataat

SEQ ID NO: 57 - DNA, 20 bp, Artificial Sequence
  Synthetic polynucleotide
cattcaaaagtaaaataata

SEQ ID NO: 58 - DNA, 20 bp, Artificial Sequence
  Synthetic polynucleotide
agtaaaataatagggtaatt

SEQ ID NO: 59 - DNA, 20 bp, Artificial Sequence
  Synthetic polynucleotide
tagtaatatcaaaattttaa

SEQ ID NO: 60 - DNA, 20 bp, Artificial Sequence
  Synthetic polynucleotide
ttagtaatatcaaaatttta

SEQ ID NO: 61 - DNA, 20 bp, Artificial Sequence
  Synthetic polynucleotide
tattgagattgttaaattta

SEQ ID NO: 62 - DNA, 20 bp, Artificial Sequence
  Synthetic polynucleotide
ttggtagatattaaaattta

SEQ ID NO: 63 - DNA, 20 bp, Artificial Sequence
  Synthetic polynucleotide
tttggtagatattaaaattt

SEQ ID NO: 64 - DNA, 20 bp, Artificial Sequence
  Synthetic polynucleotide
gaaagttcagaggcatgatt

SEQ ID NO: 65 - DNA, 20 bp, Artificial Sequence
  Synthetic polynucleotide
taacatggaggaaagttcag

SEQ ID NO: 66 - DNA, 20 bp, Artificial Sequence
  Synthetic polynucleotide
aaaaaaactaattaacatgg

SEQ ID NO: 67 - DNA, 20 bp, Artificial Sequence
  Synthetic polynucleotide
agtaaaaaaactaattaaca

SEQ ID NO: 68 - DNA, 20 bp, Artificial Sequence
  Synthetic polynucleotide
attagtttttttactaaaat

SEQ ID NO: 69 - DNA, 20 bp, Artificial Sequence
  Synthetic polynucleotide
ctctaaactattgagattgt

SEQ ID NO: 70 - DNA, 20 bp, Artificial Sequence
  Synthetic polynucleotide
ataatataatatatatatat

SEQ ID NO: 71 - DNA, 20 bp, Artificial Sequence
  Synthetic polynucleotide
gactaattaatgatcatgtg

SEQ ID NO: 72 - DNA, 20 bp, Artificial Sequence
  Synthetic polynucleotide
cttaagggaatatatgcaac

SEQ ID NO: 73 - DNA, 20 bp, Artificial Sequence
  Synthetic polynucleotide
ataataataagtatacttaa

SEQ ID NO: 74 - DNA, 20 bp, Artificial Sequence
  Synthetic polynucleotide
tataataataagtatactta

SEQ ID NO: 75 - DNA, 20 bp, Artificial Sequence
  Synthetic polynucleotide
tatataatatatatatatag

SEQ ID NO: 76 - DNA, 20 bp, Artificial Sequence
  Synthetic polynucleotide
atatataatatatatatata

SEQ ID NO: 77 - DNA, 20 bp, Artificial Sequence
  Synthetic polynucleotide
tatatataatatatatatat

SEQ ID NO: 78 - DNA, 20 bp, Artificial Sequence
  Synthetic polynucleotide
tgatcgatcgttagggggga

SEQ ID NO: 79 - DNA, 20 bp, Artificial Sequence
  Synthetic polynucleotide
atcttgatcgatcgttaggg

SEQ ID NO: 80 - DNA, 20 bp, Artificial Sequence
  Synthetic polynucleotide
catcttgatcgatcgttagg

SEQ ID NO: 81 - DNA, 20 bp, Artificial Sequence
  Synthetic polynucleotide
acatcttgatcgatcgttag

SEQ ID NO: 82 - DNA, 20 bp, Artificial Sequence
  Synthetic polynucleotide
cacatcttgatcgatcgtta

SEQ ID NO: 83 - DNA, 20 bp, Artificial Sequence
  Synthetic polynucleotide
acacatcttgatcgatcgtt

SEQ ID NO: 84 - DNA, 20 bp, Artificial Sequence
  Synthetic polynucleotide
atataagaaatatgtatgat

SEQ ID NO: 85 - DNA, 20 bp, Artificial Sequence
  Synthetic polynucleotide
atgtatgatcggaattttat

SEQ ID NO: 86 - DNA, 20 bp, Artificial Sequence
  Synthetic polynucleotide
tcaatattatatatagtatt

SEQ ID NO: 87 - DNA, 20 bp, Artificial Sequence
  Synthetic polynucleotide
aacttatatatgaatattat

SEQ ID NO: 88 - DNA, 20 bp, Artificial Sequence
  Synthetic polynucleotide
tgtgatgacaaaagtttttt

SEQ ID NO: 89 - DNA, 20 bp, Artificial Sequence
  Synthetic polynucleotide
gtgtgatgacaaaagttttt

SEQ ID NO: 90 - DNA, 20 bp, Artificial Sequence
  Synthetic polynucleotide
atttgagggagcatttacac

SEQ ID NO: 91 - DNA, 20 bp, Artificial Sequence
  Synthetic polynucleotide
ccctagtgatccttatttga

SEQ ID NO: 92 - DNA, 20 bp, Artificial Sequence
  Synthetic polynucleotide
tgtaaatgctccctcaaata

SEQ ID NO: 93 - DNA, 20 bp, Artificial Sequence
  Synthetic polynucleotide
gccctagtgatccttatttg

SEQ ID NO: 94 - DNA, 20 bp, Artificial Sequence
  Synthetic polynucleotide
tccctcaaataaggatcact

SEQ ID NO: 95 - DNA, 20 bp, Artificial Sequence
  Synthetic polynucleotide
ccctcaaataaggatcacta

SEQ ID NO: 96 - DNA, 20 bp, Artificial Sequence
  Synthetic polynucleotide
aaataaggatcactagggcc

SEQ ID NO: 97 - DNA, 20 bp, Artificial Sequence
  Synthetic polynucleotide
atgactgatcctgatgttcc

SEQ ID NO: 98 - DNA, 20 bp, Artificial Sequence
  Synthetic polynucleotide
tcactagggccaggaacatc

SEQ ID NO: 99 - DNA, 20 bp, Artificial Sequence
  Synthetic polynucleotide
tgttgtaaataatattaatt

SEQ ID NO: 100 - DNA, 20 bp, Artificial Sequence
  Synthetic polynucleotide
catgagatcctttttcacac

SEQ ID NO: 101 - DNA, 20 bp, Artificial Sequence
  Synthetic polynucleotide
aattattaccagtgtgaaaa

SEQ ID NO: 102 - DNA, 20 bp, Artificial Sequence
  Synthetic polynucleotide
ccaaaagttgaggttcatgg

SEQ ID NO: 103 - DNA, 20 bp, Artificial Sequence
  Synthetic polynucleotide
aagccaaaagttgaggttca

SEQ ID NO: 104 - DNA, 20 bp, Artificial Sequence
  Synthetic polynucleotide
tactatcaagccaaaagttg

SEQ ID NO: 105 - DNA, 20 bp, Artificial Sequence
  Synthetic polynucleotide
ccaccatgaacctcaacttt

SEQ ID NO: 106 - DNA, 20 bp, Artificial Sequence
  Synthetic polynucleotide
ttttggcttgatagtaattg

SEQ ID NO: 107 - DNA, 20 bp, Artificial Sequence
  Synthetic polynucleotide
ggcttgatagtaattgtgga

SEQ ID NO: 108 - DNA, 20 bp, Artificial Sequence
  Synthetic polynucleotide
tcaaacaagaaagtctacaa

SEQ ID NO: 109 - DNA, 20 bp, Artificial Sequence
  Synthetic polynucleotide
tgtaagtcactgtaaatttt

SEQ ID NO: 110 - DNA, 20 bp, Artificial Sequence
  Synthetic polynucleotide
gtaagtcactgtaaatttta

SEQ ID NO: 111 - DNA, 20 bp, Artificial Sequence
  Synthetic polynucleotide
gtcactgtaaattttagggt

SEQ ID NO: 112 - DNA, 20 bp, Artificial Sequence
  Synthetic polynucleotide
tggaagagtgataggagatg

SEQ ID NO: 113 - DNA, 20 bp, Artificial Sequence
  Synthetic polynucleotide
cttattattggaagagtgat

SEQ ID NO: 114 - DNA, 20 bp, Artificial Sequence
  Synthetic polynucleotide
tcagataatcctcttattat

SEQ ID NO: 115 - DNA, 20 bp, Artificial Sequence
  Synthetic polynucleotide
atcactcttccaataataag

SEQ ID NO: 116 - DNA, 20 bp, Artificial Sequence
  Synthetic polynucleotide
atttttttttaaaaaattaa

SEQ ID NO: 117 - DNA, 20 bp, Artificial Sequence
  Synthetic polynucleotide
tgtgtcctctctataaataa

SEQ ID NO: 118 - DNA, 20 bp, Artificial Sequence
  Synthetic polynucleotide
ttgtgtcctctctataaata

SEQ ID NO: 119 - DNA, 20 bp, Artificial Sequence
  Synthetic polynucleotide
tatatcccttatttatagag

SEQ ID NO: 120 - DNA, 20 bp, Artificial Sequence
  Synthetic polynucleotide
gagatgtttaatatattaag

SEQ ID NO: 121 - DNA, 20 bp, Artificial Sequence
  Synthetic polynucleotide
agatgtttaatatattaagt

SEQ ID NO: 122 - DNA, 20 bp, Artificial Sequence
  Synthetic polynucleotide
gatgtttaatatattaagtg

SEQ ID NO: 123 - DNA, 20 bp, Artificial Sequence
  Synthetic polynucleotide
aactaaaataatttaaatga

SEQ ID NO: 124 - DNA, 20 bp, Artificial Sequence
  Synthetic polynucleotide
aatgaaggaatagtataaaa

SEQ ID NO: 125 - DNA, 20 bp, Artificial Sequence
  Synthetic polynucleotide
atgaaggaatagtataaaaa

SEQ ID NO: 126 - DNA, 20 bp, Artificial Sequence
  Synthetic polynucleotide
aaagggtcatcatcttcaaa

SEQ ID NO: 127 - DNA, 20 bp, Artificial Sequence
  Synthetic polynucleotide
tttttatcttatcatgttgt

SEQ ID NO: 128 - DNA, 20 bp, Artificial Sequence
  Synthetic polynucleotide
tgtaagagaacagctcttaa

SEQ ID NO: 129 - DNA, 20 bp, Artificial Sequence
  Synthetic polynucleotide
ttgtaagagaacagctctta

SEQ ID NO: 130 - DNA, 20 bp, Artificial Sequence
  Synthetic polynucleotide
actagttttagtacgatgat

SEQ ID NO: 131 - DNA, 20 bp, Artificial Sequence
  Synthetic polynucleotide
aataagagattaaccgtgcg

SEQ ID NO: 132 - DNA, 20 bp, Artificial Sequence
  Synthetic polynucleotide
tagttttctttttcctcgca

SEQ ID NO: 133 - DNA, 20 bp, Artificial Sequence
  Synthetic polynucleotide
tattatatatatgtcattat

SEQ ID NO: 134 - DNA, 20 bp, Artificial Sequence
  Synthetic polynucleotide
atatatatgtcattattggc

SEQ ID NO: 135 - DNA, 20 bp, Artificial Sequence
  Synthetic polynucleotide
tatatatgtcattattggca

SEQ ID NO: 136 - DNA, 20 bp, Artificial Sequence
  Synthetic polynucleotide
attgaagtatacaatagcca

SEQ ID NO: 137 - DNA, 20 bp, Artificial Sequence
  Synthetic polynucleotide
ttgaagtatacaatagccac

SEQ ID NO: 138 - DNA, 20 bp, Artificial Sequence
  Synthetic polynucleotide
tgaagtatacaatagccacg

SEQ ID NO: 139 - DNA, 20 bp, Artificial Sequence
  Synthetic polynucleotide
caacaatctgggtctccccg

SEQ ID NO: 140 - DNA, 20 bp, Artificial Sequence
  Synthetic polynucleotide
ttttctgaagacaacaatct

SEQ ID NO: 141 - DNA, 20 bp, Artificial Sequence
  Synthetic polynucleotide
attttctgaagacaacaatc

SEQ ID NO: 142 - DNA, 20 bp, Artificial Sequence
  Synthetic polynucleotide
tgagagactgttttaacaca

SEQ ID NO: 143 - DNA, 20 bp, Artificial Sequence
  Synthetic polynucleotide
ttaaaacagtctctcaaagc

SEQ ID NO: 144 - DNA, 20 bp, Artificial Sequence
  Synthetic polynucleotide
ttcatcttattcaagcaaag

SEQ ID NO: 145 - DNA, 20 bp, Artificial Sequence
  Synthetic polynucleotide
aatctatgaatccccaccac

SEQ ID NO: 146 - DNA, 20 bp, Artificial Sequence
  Synthetic polynucleotide
aagctccaaaaccggtggtg

SEQ ID NO: 147 - DNA, 20 bp, Artificial Sequence
  Synthetic polynucleotide
gaagctccaaaaccggtggt

SEQ ID NO: 148 - DNA, 20 bp, Artificial Sequence
  Synthetic polynucleotide
tgaagctccaaaaccggtgg

SEQ ID NO: 149 - DNA, 20 bp, Artificial Sequence
  Synthetic polynucleotide
ttatgaagctccaaaaccgg

SEQ ID NO: 150 - DNA, 20 bp, Artificial Sequence
  Synthetic polynucleotide
tgaatccccaccaccggttt

SEQ ID NO: 151 - DNA, 20 bp, Artificial Sequence
  Synthetic polynucleotide
gagttatgaagctccaaaac

SEQ ID NO: 152 - DNA, 20 bp, Artificial Sequence
  Synthetic polynucleotide
atatatttataaacgaaaac

SEQ ID NO: 153 - DNA, 20 bp, Artificial Sequence
  Synthetic polynucleotide
taacgaaataaatatctaaa

SEQ ID NO: 154 - DNA, 20 bp, Artificial Sequence
  Synthetic polynucleotide
gtttttttcttttggtagtg

SEQ ID NO: 155 - DNA, 20 bp, Artificial Sequence
  Synthetic polynucleotide
tgcattatgtttttttcttt

SEQ ID NO: 156 - DNA, 20 bp, Artificial Sequence
  Synthetic polynucleotide
aaagaaaaaaacataatgca

SEQ ID NO: 157 - DNA, 20 bp, Artificial Sequence
  Synthetic polynucleotide
cacacatatatacgatagtg

SEQ ID NO: 158 - DNA, 20 bp, Artificial Sequence
  Synthetic polynucleotide
ggacatgaaatactcaccaa

SEQ ID NO: 159 - DNA, 20 bp, Artificial Sequence
  Synthetic polynucleotide
ggtacaactaatgtatcctt

SEQ ID NO: 160 - DNA, 20 bp, Artificial Sequence
  Synthetic polynucleotide
aaggatacattagttgtacc

SEQ ID NO: 161 - DNA, 20 bp, Artificial Sequence
  Synthetic polynucleotide
aggatagtcacagacattcc

SEQ ID NO: 162 - DNA, 20 bp, Artificial Sequence
  Synthetic polynucleotide
tggaatgtctgtgactatcc

SEQ ID NO: 163 - DNA, 20 bp, Artificial Sequence
  Synthetic polynucleotide
ataatacgtgtattctttcc

SEQ ID NO: 164 - DNA, 20 bp, Artificial Sequence
  Synthetic polynucleotide
gtgatcctttccattaatat

SEQ ID NO: 165 - DNA, 20 bp, Artificial Sequence
  Synthetic polynucleotide
aattaaatatccaatattaa

SEQ ID NO: 166 - DNA, 20 bp, Artificial Sequence
  Synthetic polynucleotide
aatatccaatattaatggaa

SEQ ID NO: 167 - DNA, 20 bp, Artificial Sequence
  Synthetic polynucleotide
atatataaatatacaagaag

SEQ ID NO: 168 - DNA, 20 bp, Artificial Sequence
  Synthetic polynucleotide
attatattacaaaccagtga

SEQ ID NO: 169 - DNA, 20 bp, Artificial Sequence
  Synthetic polynucleotide
acttaaaagaacaccttcac

SEQ ID NO: 170 - DNA, 20 bp, Artificial Sequence
  Synthetic polynucleotide
gtgaaggtgttcttttaagt

SEQ ID NO: 171 - DNA, 20 bp, Artificial Sequence
  Synthetic polynucleotide
tgaaggtgttcttttaagta

SEQ ID NO: 172 - DNA, 20 bp, Artificial Sequence
  Synthetic polynucleotide
tcttttaagtagggatcact

SEQ ID NO: 173 - DNA, 20 bp, Artificial Sequence
  Synthetic polynucleotide
cttttaagtagggatcacta

SEQ ID NO: 174 - DNA, 20 bp, Artificial Sequence
  Synthetic polynucleotide
aagtagggatcactagggct

SEQ ID NO: 175 - DNA, 20 bp, Artificial Sequence
  Synthetic polynucleotide
tcactagggcttggagcatc

SEQ ID NO: 176 - DNA, 20 bp, Artificial Sequence
  Synthetic polynucleotide
cacttatttcttgaagatta

SEQ ID NO: 177 - DNA, 20 bp, Artificial Sequence
  Synthetic polynucleotide
cataatcttcaagaaataag

SEQ ID NO: 178 - DNA, 20 bp, Artificial Sequence
  Synthetic polynucleotide
tcaagaaataagtggaaaaa

SEQ ID NO: 179 - DNA, 20 bp, Artificial Sequence
  Synthetic polynucleotide
caagaaataagtggaaaaaa

SEQ ID NO: 180 - DNA, 20 bp, Artificial Sequence
  Synthetic polynucleotide
aaacctcgagtagatattgg

SEQ ID NO: 181 - DNA, 20 bp, Artificial Sequence
  Synthetic polynucleotide
tctaaacctcgagtagatat

SEQ ID NO: 182 - DNA, 20 bp, Artificial Sequence
  Synthetic polynucleotide
tctccaccaatatctactcg

SEQ ID NO: 183 - DNA, 20 bp, Artificial Sequence
  Synthetic polynucleotide
ggtttagacaaaatattaga

SEQ ID NO: 184 - DNA, 20 bp, Artificial Sequence
  Synthetic polynucleotide
cctgataagcaagtttgtaa

SEQ ID NO: 185 - DNA, 20 bp, Artificial Sequence
  Synthetic polynucleotide
ccattacaaacttgcttatc

SEQ ID NO: 186 - DNA, 20 bp, Artificial Sequence
  Synthetic polynucleotide
cattacaaacttgcttatca

SEQ ID NO: 187 - DNA, 20 bp, Artificial Sequence
  Synthetic polynucleotide
atgacagtcatttttacact

SEQ ID NO: 188 - DNA, 20 bp, Artificial Sequence
  Synthetic polynucleotide
ggcaagagtaataggagatg

SEQ ID NO: 189 - DNA, 20 bp, Artificial Sequence
  Synthetic polynucleotide
ttaagtgtggcaagagtaat

SEQ ID NO: 190 - DNA, 20 bp, Artificial Sequence
  Synthetic polynucleotide
agcaatacaaactttaagtg

SEQ ID NO: 191 - DNA, 20 bp, Artificial Sequence
  Synthetic polynucleotide
tttatataataataatgtca

SEQ ID NO: 192 - DNA, 20 bp, Artificial Sequence
  Synthetic polynucleotide
aatagatcagatatatgata

SEQ ID NO: 193 - DNA, 20 bp, Artificial Sequence
  Synthetic polynucleotide
agatatatgataaggatgag

SEQ ID NO: 194 - DNA, 20 bp, Artificial Sequence
  Synthetic polynucleotide
gtagtagtagagatcgagtt

SEQ ID NO: 195 - DNA, 20 bp, Artificial Sequence
  Synthetic polynucleotide
agatcgagttaggtgatcaa

SEQ ID NO: 196 - DNA, 20 bp, Artificial Sequence
  Synthetic polynucleotide
gatcgagttaggtgatcaat

SEQ ID NO: 197 - DNA, 20 bp, Artificial Sequence
  Synthetic polynucleotide
tgggtatttataagccagct

SEQ ID NO: 198 - DNA, 20 bp, Artificial Sequence
  Synthetic polynucleotide
gggtatttataagccagcta

SEQ ID NO: 199 - DNA, 20 bp, Artificial Sequence
  Synthetic polynucleotide
aaattccaacttgccctagc

SEQ ID NO: 200 - DNA, 20 bp, Artificial Sequence
  Synthetic polynucleotide
ataagccagctagggcaagt

SEQ ID NO: 201 - DNA, 20 bp, Artificial Sequence
  Synthetic polynucleotide
ttatttgaatagcgtagtag

SEQ ID NO: 202 - DNA, 20 bp, Artificial Sequence
  Synthetic polynucleotide
agcgtagtagtggctgctct

SEQ ID NO: 203 - DNA, 20 bp, Artificial Sequence
  Synthetic polynucleotide
gcgtagtagtggctgctctt

SEQ ID NO: 204 - DNA, 20 bp, Artificial Sequence
  Synthetic polynucleotide
ggctgctcttgggaaatgcc

SEQ ID NO: 205 - DNA, 20 bp, Artificial Sequence
  Synthetic polynucleotide
aataaagcaaatattaaacc

SEQ ID NO: 206 - DNA, 20 bp, Artificial Sequence
  Synthetic polynucleotide
gtaaaaagttaaaataccaa

SEQ ID NO: 207 - DNA, 20 bp, Artificial Sequence
  Synthetic polynucleotide
gtgatgttttcaaattcctt

SEQ ID NO: 208 - DNA, 20 bp, Artificial Sequence
  Synthetic polynucleotide
catacgtgtcgaactaggaa

SEQ ID NO: 209 - DNA, 20 bp, Artificial Sequence
  Synthetic polynucleotide
acaaccatacgtgtcgaact

SEQ ID NO: 210 - DNA, 20 bp, Artificial Sequence
  Synthetic polynucleotide
cattcctagttcgacacgta

SEQ ID NO: 211 - DNA, 20 bp, Artificial Sequence
  Synthetic polynucleotide
ttgttgatgttgaaagaagt

SEQ ID NO: 212 - DNA, 20 bp, Artificial Sequence
  Synthetic polynucleotide
agaatatatatatatattag

SEQ ID NO: 213 - DNA, 20 bp, Artificial Sequence
  Synthetic polynucleotide
tatatatatatattagtggt

SEQ ID NO: 214 - DNA, 20 bp, Artificial Sequence
  Synthetic polynucleotide
tatatatattagtggtaggt

SEQ ID NO: 215 - DNA, 20 bp, Artificial Sequence
  Synthetic polynucleotide
gagctagcgtcttctagctg

SEQ ID NO: 216 - DNA, 20 bp, Artificial Sequence
  Synthetic polynucleotide
tctctgagcgttgaaataaa

SEQ ID NO: 217 - DNA, 20 bp, Artificial Sequence
  Synthetic polynucleotide
ttgaaataaacggcagcgac

SEQ ID NO: 218 - DNA, 20 bp, Artificial Sequence
  Synthetic polynucleotide
cggcagcgacaggaagaccg

SEQ ID NO: 219 - DNA, 20 bp, Artificial Sequence
  Synthetic polynucleotide
ttcgctgctcaaaacgacct

SEQ ID NO: 220 - DNA, 20 bp, Artificial Sequence
  Synthetic polynucleotide
ggtgagtataccaagctcaa

SEQ ID NO: 221 - DNA, 20 bp, Artificial Sequence
  Synthetic polynucleotide
ttgaaatgatcctttgagct

SEQ ID NO: 222 - DNA, 20 bp, Artificial Sequence
  Synthetic polynucleotide
ttggtatactcaccgagtct

SEQ ID NO: 223 - DNA, 20 bp, Artificial Sequence
  Synthetic polynucleotide
caaacagaaatgccgagact

SEQ ID NO: 224 - DNA, 20 bp, Artificial Sequence
  Synthetic polynucleotide
agccaaatatagggattcac

SEQ ID NO: 225 - DNA, 20 bp, Artificial Sequence
  Synthetic polynucleotide
aacctgtgaatccctatatt

SEQ ID NO: 226 - DNA, 20 bp, Artificial Sequence
  Synthetic polynucleotide
agagcccaaagccaaatata

SEQ ID NO: 227 - DNA, 20 bp, Artificial Sequence
  Synthetic polynucleotide
gagagcccaaagccaaatat

SEQ ID NO: 228 - DNA, 20 bp, Artificial Sequence
  Synthetic polynucleotide
tgaatccctatatttggctt

SEQ ID NO: 229 - DNA, 20 bp, Artificial Sequence
  Synthetic polynucleotide
gaatccctatatttggcttt

SEQ ID NO: 230 - DNA, 20 bp, Artificial Sequence
  Synthetic polynucleotide
tatgtacacaggaagggaag

SEQ ID NO: 231 - DNA, 20 bp, Artificial Sequence
  Synthetic polynucleotide
aatggatatgtacacaggaa

SEQ ID NO: 232 - DNA, 20 bp, Artificial Sequence
  Synthetic polynucleotide
aaatggatatgtacacagga

SEQ ID NO: 233 - DNA, 20 bp, Artificial Sequence
  Synthetic polynucleotide
attaaaatggatatgtacac

SEQ ID NO: 234 - DNA, 20 bp, Artificial Sequence
  Synthetic polynucleotide
ttataaatataaaattaaaa

SEQ ID NO: 235 - DNA, 20 bp, Artificial Sequence
  Synthetic polynucleotide
tatacatatataattaataa

SEQ ID NO: 236 - DNA, 20 bp, Artificial Sequence
  Synthetic polynucleotide
ggcaccacagacgctacatt

SEQ ID NO: 237 - DNA, 20 bp, Artificial Sequence
  Synthetic polynucleotide
attaccaaatgtagcgtctg

SEQ ID NO: 238 - DNA, 20 bp, Artificial Sequence
  Synthetic polynucleotide
aatgtagcgtctgtggtgcc

SEQ ID NO: 239 - DNA, 20 bp, Artificial Sequence
  Synthetic polynucleotide
aggattgtgacagacatacc

SEQ ID NO: 240 - DNA, 20 bp, Artificial Sequence
  Synthetic polynucleotide
ttcttctttgaaacatggac

SEQ ID NO: 241 - DNA, 20 bp, Artificial Sequence
  Synthetic polynucleotide
ttattttcttctttgaaaca

SEQ ID NO: 242 - DNA, 20 bp, Artificial Sequence
  Synthetic polynucleotide
agaaaataattattacaatc

SEQ ID NO: 243 - DNA, 20 bp, Artificial Sequence
  Synthetic polynucleotide
tattacaatcaggatatctt

SEQ ID NO: 244 - DNA, 20 bp, Artificial Sequence
  Synthetic polynucleotide
tttaattaacaaactatttt

SEQ ID NO: 245 - DNA, 20 bp, Artificial Sequence
  Synthetic polynucleotide
atgtcagtaagattataatt

SEQ ID NO: 246 - DNA, 20 bp, Artificial Sequence
  Synthetic polynucleotide
attataatcttactgacatt

SEQ ID NO: 247 - DNA, 20 bp, Artificial Sequence
  Synthetic polynucleotide
actatttattataccagtga

SEQ ID NO: 248 - DNA, 20 bp, Artificial Sequence
  Synthetic polynucleotide
acttaagggagcaccttcac

SEQ ID NO: 249 - DNA, 20 bp, Artificial Sequence
  Synthetic polynucleotide
cccaagtgatccttacttaa

SEQ ID NO: 250 - DNA, 20 bp, Artificial Sequence
  Synthetic polynucleotide
tgaaggtgctcccttaagta

SEQ ID NO: 251 - DNA, 20 bp, Artificial Sequence
  Synthetic polynucleotide
gcccaagtgatccttactta

SEQ ID NO: 252 - DNA, 20 bp, Artificial Sequence
  Synthetic polynucleotide
tcccttaagtaaggatcact

SEQ ID NO: 253 - DNA, 20 bp, Artificial Sequence
  Synthetic polynucleotide
cccttaagtaaggatcactt

SEQ ID NO: 254 - DNA, 20 bp, Artificial Sequence
  Synthetic polynucleotide
aagtaaggatcacttgggcc

SEQ ID NO: 255 - DNA, 20 bp, Artificial Sequence
  Synthetic polynucleotide
atgacagatccagatgttcc

SEQ ID NO: 256 - DNA, 20 bp, Artificial Sequence
  Synthetic polynucleotide
tcacttgggccaggaacatc

SEQ ID NO: 257 - DNA, 20 bp, Artificial Sequence
  Synthetic polynucleotide
ctctctctttattttatttt

SEQ ID NO: 258 - DNA, 20 bp, Artificial Sequence
  Synthetic polynucleotide
aattaatgttgttctttttt

SEQ ID NO: 259 - DNA, 20 bp, Artificial Sequence
  Synthetic polynucleotide
agatctctaaatgatgaaca

SEQ ID NO: 260 - DNA, 20 bp, Artificial Sequence
  Synthetic polynucleotide
tttttttattagtgtgaaga

SEQ ID NO: 261 - DNA, 20 bp, Artificial Sequence
  Synthetic polynucleotide
aaacctagggttgagattca

SEQ ID NO: 262 - DNA, 20 bp, Artificial Sequence
  Synthetic polynucleotide
gctccttgaatctcaaccct

SEQ ID NO: 263 - DNA, 20 bp, Artificial Sequence
  Synthetic polynucleotide
ttgaatctcaaccctaggtt

SEQ ID NO: 264 - DNA, 20 bp, Artificial Sequence
  Synthetic polynucleotide
agttgtcatctccaaaccta

SEQ ID NO: 265 - DNA, 20 bp, Artificial Sequence
  Synthetic polynucleotide
cagttgtcatctccaaacct

SEQ ID NO: 266 - DNA, 20 bp, Artificial Sequence
  Synthetic polynucleotide
ggtttggagatgacaactga

SEQ ID NO: 267 - DNA, 20 bp, Artificial Sequence
  Synthetic polynucleotide
aacaagaagcaagtctgtaa

SEQ ID NO: 268 - DNA, 20 bp, Artificial Sequence
  Synthetic polynucleotide
ataagagacagacattttta

SEQ ID NO: 269 - DNA, 20 bp, Artificial Sequence
  Synthetic polynucleotide
gagacagacatttttatgga

SEQ ID NO: 270 - DNA, 20 bp, Artificial Sequence
  Synthetic polynucleotide
cttgtggttggaagagtgat

SEQ ID NO: 271 - DNA, 20 bp, Artificial Sequence
  Synthetic polynucleotide
atgttagagcctcttgtggt

SEQ ID NO: 272 - DNA, 20 bp, Artificial Sequence
  Synthetic polynucleotide
atcactcttccaaccacaag

SEQ ID NO: 273 - DNA, 20 bp, Artificial Sequence
  Synthetic polynucleotide
aagaatgttagagcctcttg

SEQ ID NO: 274 - DNA, 20 bp, Artificial Sequence
  Synthetic polynucleotide
tattactataaatataatta

SEQ ID NO: 275 - DNA, 20 bp, Artificial Sequence
  Synthetic polynucleotide
atatattttaagtacttaat

SEQ ID NO: 276 - DNA, 20 bp, Artificial Sequence
  Synthetic polynucleotide
tttatatatgtgtttgtgtg

SEQ ID NO: 277 - DNA, 20 bp, Artificial Sequence
  Synthetic polynucleotide
aattattacttagttcgtga

SEQ ID NO: 278 - DNA, 20 bp, Artificial Sequence
  Synthetic polynucleotide
ttacttagttcgtgatggaa

SEQ ID NO: 279 - DNA, 20 bp, Artificial Sequence
  Synthetic polynucleotide
gttcgtgatggaatggaact

SEQ ID NO: 280 - DNA, 20 bp, Artificial Sequence
  Synthetic polynucleotide
tgtagacactataaatacat

SEQ ID NO: 281 - DNA, 20 bp, Artificial Sequence
  Synthetic polynucleotide
atgtagacactataaataca

SEQ ID NO: 282 - DNA, 20 bp, Artificial Sequence
  Synthetic polynucleotide
tagtgtctacatgatatctt

SEQ ID NO: 283 - DNA, 20 bp, Artificial Sequence
  Synthetic polynucleotide
taaaagtaaagttacaatta

SEQ ID NO: 284 - DNA, 20 bp, Artificial Sequence
  Synthetic polynucleotide
atattaatatataacaagtt

SEQ ID NO: 285 - DNA, 20 bp, Artificial Sequence
  Synthetic polynucleotide
aataattaattaagaatata

SEQ ID NO: 286 - DNA, 20 bp, Artificial Sequence
  Synthetic polynucleotide
aagaatatatggctagagat

SEQ ID NO: 287 - DNA, 20 bp, Artificial Sequence
  Synthetic polynucleotide
agaatatatggctagagata

SEQ ID NO: 288 - DNA, 20 bp, Artificial Sequence
  Synthetic polynucleotide
tagagatagggaccctcttg

SEQ ID NO: 289 - DNA, 20 bp, Artificial Sequence
  Synthetic polynucleotide
ttactctaccaaccacaaga

SEQ ID NO: 290 - DNA, 20 bp, Artificial Sequence
  Synthetic polynucleotide
attactctaccaaccacaag

SEQ ID NO: 291 - DNA, 20 bp, Artificial Sequence
  Synthetic polynucleotide
gatagggaccctcttgtggt

SEQ ID NO: 292 - DNA, 20 bp, Artificial Sequence
  Synthetic polynucleotide
cttgtggttggtagagtaat

SEQ ID NO: 293 - DNA, 20 bp, Artificial Sequence
  Synthetic polynucleotide
tagagtaataggagatgttt

SEQ ID NO: 294 - DNA, 20 bp, Artificial Sequence
  Synthetic polynucleotide
atgttttggatccttttaca

SEQ ID NO: 295 - DNA, 20 bp, Artificial Sequence
  Synthetic polynucleotide
agagagactgaccttgtaaa

SEQ ID NO: 296 - DNA, 20 bp, Artificial Sequence
  Synthetic polynucleotide
gtctctcttagagtgagtta

SEQ ID NO: 297 - DNA, 20 bp, Artificial Sequence
  Synthetic polynucleotide
agtgagttatggtaatagag

SEQ ID NO: 298 - DNA, 20 bp, Artificial Sequence
  Synthetic polynucleotide
ggtaatagagaggtcaacaa

SEQ ID NO: 299 - DNA, 20 bp, Artificial Sequence
  Synthetic polynucleotide
ggttggttaacaatttggga

SEQ ID NO: 300 - DNA, 20 bp, Artificial Sequence
  Synthetic polynucleotide
acgaggttggttaacaattt

SEQ ID NO: 301 - DNA, 20 bp, Artificial Sequence
  Synthetic polynucleotide
cacgaggttggttaacaatt

SEQ ID NO: 302 - DNA, 20 bp, Artificial Sequence
  Synthetic polynucleotide
caccaatatcaacacgaggt

SEQ ID NO: 303 - DNA, 20 bp, Artificial Sequence
  Synthetic polynucleotide
tcaccaccaatatcaacacg

SEQ ID NO: 304 - DNA, 20 bp, Artificial Sequence
  Synthetic polynucleotide
aaccaacctcgtgttgatat

SEQ ID NO: 305 - DNA, 20 bp, Artificial Sequence
  Synthetic polynucleotide
caacctcgtgttgatattgg

SEQ ID NO: 306 - DNA, 20 bp, Artificial Sequence
  Synthetic polynucleotide
atattggtggtgatgaccta

SEQ ID NO: 307 - DNA, 20 bp, Artificial Sequence
  Synthetic polynucleotide
ccaaagtgtagaaggtcctt

SEQ ID NO: 308 - DNA, 20 bp, Artificial Sequence
  Synthetic polynucleotide
ttaatttaccaaagtgtaga

SEQ ID NO: 309 - DNA, 20 bp, Artificial Sequence
  Synthetic polynucleotide
cctaaggaccttctacactt

SEQ ID NO: 310 - DNA, 20 bp, Artificial Sequence
  Synthetic polynucleotide
tgaaatcattatgaatattg

SEQ ID NO: 311 - DNA, 20 bp, Artificial Sequence
  Synthetic polynucleotide
catatattgaaaattattac

SEQ ID NO: 312 - DNA, 20 bp, Artificial Sequence
  Synthetic polynucleotide
ttgaaaattattacaggtca

SEQ ID NO: 313 - DNA, 20 bp, Artificial Sequence
  Synthetic polynucleotide
aaaattattacaggtcatgg

SEQ ID NO: 314 - DNA, 20 bp, Artificial Sequence
  Synthetic polynucleotide
attacaggtcatggtggatc

SEQ ID NO: 315 - DNA, 20 bp, Artificial Sequence
  Synthetic polynucleotide
ttgctagggctaggagcatc

SEQ ID NO: 316 - DNA, 20 bp, Artificial Sequence
  Synthetic polynucleotide
agattggggttgctagggct

SEQ ID NO: 317 - DNA, 20 bp, Artificial Sequence
  Synthetic polynucleotide
cccttagattggggttgcta

SEQ ID NO: 318 - DNA, 20 bp, Artificial Sequence
  Synthetic polynucleotide
tcccttagattggggttgct

SEQ ID NO: 319 - DNA, 20 bp, Artificial Sequence
  Synthetic polynucleotide
gcaaatactcccttagattg

SEQ ID NO: 320 - DNA, 20 bp, Artificial Sequence
  Synthetic polynucleotide
gccctagcaaccccaatcta

SEQ ID NO: 321 - DNA, 20 bp, Artificial Sequence
  Synthetic polynucleotide
tgcaaatactcccttagatt

SEQ ID NO: 322 - DNA, 20 bp, Artificial Sequence
  Synthetic polynucleotide
ccctagcaaccccaatctaa

SEQ ID NO: 323 - DNA, 20 bp, Artificial Sequence
  Synthetic polynucleotide
atgcaaatactcccttagat

SEQ ID NO: 324 - DNA, 20 bp, Artificial Sequence
  Synthetic polynucleotide
atctaagggagtatttgcat

SEQ ID NO: 325 - DNA, 20 bp, Artificial Sequence
  Synthetic polynucleotide
atattattattaaatagatg

SEQ ID NO: 326 - DNA, 20 bp, Artificial Sequence
  Synthetic polynucleotide
tattattattaaatagatga

SEQ ID NO: 327 - DNA, 20 bp, Artificial Sequence
  Synthetic polynucleotide
tttaattttgtataaaactt

SEQ ID NO: 328 - DNA, 20 bp, Artificial Sequence
  Synthetic polynucleotide
ttcatgcacacaacacatgt

SEQ ID NO: 329 - DNA, 20 bp, Artificial Sequence
  Synthetic polynucleotide
caaaaagtaaagacatattt

SEQ ID NO: 330 - DNA, 20 bp, Artificial Sequence
  Synthetic polynucleotide
caaaatatgtctttactttt

SEQ ID NO: 331 - DNA, 20 bp, Artificial Sequence
  Synthetic polynucleotide
cattttataaagatgttagt

SEQ ID NO: 332 - DNA, 20 bp, Artificial Sequence
  Synthetic polynucleotide
attttataaagatgttagtt

SEQ ID NO: 333 - DNA, 20 bp, Artificial Sequence
  Synthetic polynucleotide
ttttagtgtcagttttgaat

SEQ ID NO: 334 - DNA, 20 bp, Artificial Sequence
  Synthetic polynucleotide
acaaaattctgtaattatta

SEQ ID NO: 335 - DNA, 20 bp, Artificial Sequence
  Synthetic polynucleotide
tacaaaattctgtaattatt

SEQ ID NO: 336 - DNA, 20 bp, Artificial Sequence
  Synthetic polynucleotide
acaaattaaaacaagcttta

SEQ ID NO: 337 - DNA, 20 bp, Artificial Sequence
  Synthetic polynucleotide
aacaaattaaaacaagcttt

SEQ ID NO: 338 - DNA, 20 bp, Artificial Sequence
  Synthetic polynucleotide
tttaatttgttaaagtgact

SEQ ID NO: 339 - DNA, 20 bp, Artificial Sequence
  Synthetic polynucleotide
aacatgtaaaaagaatttaa

SEQ ID NO: 340 - DNA, 20 bp, Artificial Sequence
  Synthetic polynucleotide
aaacatgtaaaaagaattta

SEQ ID NO: 341 - DNA, 20 bp, Artificial Sequence
  Synthetic polynucleotide
gctagcatatatggaatttg

SEQ ID NO: 342 - DNA, 20 bp, Artificial Sequence
  Synthetic polynucleotide
atttatatagctagcatata

SEQ ID NO: 343 - DNA, 20 bp, Artificial Sequence
  Synthetic polynucleotide
tagctatataaatataaata

SEQ ID NO: 344 - DNA, 20 bp, Artificial Sequence
  Synthetic polynucleotide
atataaatataaatatggaa

SEQ ID NO: 345 - DNA, 20 bp, Artificial Sequence
  Synthetic polynucleotide
ataaatatggaaaggatata

SEQ ID NO: 346 - DNA, 20 bp, Artificial Sequence
  Synthetic polynucleotide
taaatatggaaaggatatat

SEQ ID NO: 347 - DNA, 20 bp, Artificial Sequence
  Synthetic polynucleotide
aaagctgatgagaaagaatg

SEQ ID NO: 348 - DNA, 20 bp, Artificial Sequence
  Synthetic polynucleotide
tgatgagaaagaatgtggtt

SEQ ID NO: 349 - DNA, 20 bp, Artificial Sequence
  Synthetic polynucleotide
gatgagaaagaatgtggttt

SEQ ID NO: 350 - DNA, 20 bp, Artificial Sequence
  Synthetic polynucleotide
atgagaaagaatgtggtttg

SEQ ID NO: 351 - DNA, 20 bp, Artificial Sequence
  Synthetic polynucleotide
ggatgaattttgaatgatga

SEQ ID NO: 352 - DNA, 20 bp, Artificial Sequence
  Synthetic polynucleotide
gatgaattttgaatgatgaa

SEQ ID NO: 353 - DNA, 20 bp, Artificial Sequence
  Synthetic polynucleotide
ttttgaatgatgaagggatg

SEQ ID NO: 354 - DNA, 20 bp, Artificial Sequence
  Synthetic polynucleotide
aagggatgaggctgtgtgtg

SEQ ID NO: 355 - DNA, 20 bp, Artificial Sequence
  Synthetic polynucleotide
gggacatgctatagctagca

SEQ ID NO: 356 - DNA, 20 bp, Artificial Sequence
  Synthetic polynucleotide
ggggacatgctatagctagc

SEQ ID NO: 357 - DNA, 20 bp, Artificial Sequence
  Synthetic polynucleotide
ttttaatggtgggacaaaag

SEQ ID NO: 358 - DNA, 20 bp, Artificial Sequence
  Synthetic polynucleotide
attttaatggtgggacaaaa

SEQ ID NO: 359 - DNA, 20 bp, Artificial Sequence
  Synthetic polynucleotide
cattttaatggtgggacaaa

SEQ ID NO: 360 - DNA, 20 bp, Artificial Sequence
  Synthetic polynucleotide
gaggtggccattttaatggt

SEQ ID NO: 361 - DNA, 20 bp, Artificial Sequence
  Synthetic polynucleotide
tgaggtggccattttaatgg

SEQ ID NO: 362 - DNA, 20 bp, Artificial Sequence
  Synthetic polynucleotide
cttttgtcccaccattaaaa

SEQ ID NO: 363 - DNA, 20 bp, Artificial Sequence
  Synthetic polynucleotide
gtgtgaggtggccattttaa

SEQ ID NO: 364 - DNA, 20 bp, Artificial Sequence
  Synthetic polynucleotide
aaaaccttcttagtgtgagg

SEQ ID NO: 365 - DNA, 20 bp, Artificial Sequence
  Synthetic polynucleotide
gtgaaaaccttcttagtgtg

SEQ ID NO: 366 - DNA, 20 bp, Artificial Sequence
  Synthetic polynucleotide
atggccacctcacactaaga

SEQ ID NO: 367 - DNA, 20 bp, Artificial Sequence
  Synthetic polynucleotide
atatatacatacacatgtat

SEQ ID NO: 368 - DNA, 20 bp, Artificial Sequence
  Synthetic polynucleotide
tatatacatacacatgtata

SEQ ID NO: 369 - DNA, 20 bp, Artificial Sequence
  Synthetic polynucleotide
ctatgttcgaattcaaattc

SEQ ID NO: 370 - DNA, 20 bp, Artificial Sequence
  Synthetic polynucleotide
tctatgttcgaattcaaatt

SEQ ID NO: 371 - DNA, 20 bp, Artificial Sequence
  Synthetic polynucleotide
ttcgaacatagactcagatt

SEQ ID NO: 372 - DNA, 20 bp, Artificial Sequence
  Synthetic polynucleotide
agggttcagggtcgaattta

SEQ ID NO: 373 - DNA, 20 bp, Artificial Sequence
  Synthetic polynucleotide
cagggttcagggtcgaattt

SEQ ID NO: 374 - DNA, 20 bp, Artificial Sequence
  Synthetic polynucleotide
agttcgtgtttcagggttca

SEQ ID NO: 375 - DNA, 20 bp, Artificial Sequence
  Synthetic polynucleotide
aagttcgtgtttcagggttc

SEQ ID NO: 376 - DNA, 20 bp, Artificial Sequence
  Synthetic polynucleotide
gagtctaagttcgtgtttca

SEQ ID NO: 377 - DNA, 20 bp, Artificial Sequence
  Synthetic polynucleotide
tgagtctaagttcgtgtttc

SEQ ID NO: 378 - DNA, 20 bp, Artificial Sequence
  Synthetic polynucleotide
cacgaacttagactcagacc

SEQ ID NO: 379 - DNA, 20 bp, Artificial Sequence
  Synthetic polynucleotide
cttagactcagacctggacc

SEQ ID NO: 380 - DNA, 20 bp, Artificial Sequence
  Synthetic polynucleotide
ttgggtcaaggtccaggtcc

SEQ ID NO: 381 - DNA, 20 bp, Artificial Sequence
  Synthetic polynucleotide
cggggtttgggtcaaggtcc

SEQ ID NO: 382 - DNA, 20 bp, Artificial Sequence
  Synthetic polynucleotide
tcgggtcggggtttgggtca

SEQ ID NO: 383 - DNA, 20 bp, Artificial Sequence
  Synthetic polynucleotide
cggggttcgggtcggggttt

SEQ ID NO: 384 - DNA, 20 bp, Artificial Sequence
  Synthetic polynucleotide
tcggggttcgggtcggggtt

SEQ ID NO: 385 - DNA, 20 bp, Artificial Sequence
  Synthetic polynucleotide
tcgagtcggggttcgggtcg

SEQ ID NO: 386 - DNA, 20 bp, Artificial Sequence
  Synthetic polynucleotide
ttcgagtcggggttcgggtc

SEQ ID NO: 387 - DNA, 20 bp, Artificial Sequence
  Synthetic polynucleotide
gttcgagtcggggttcgggt

SEQ ID NO: 388 - DNA, 20 bp, Artificial Sequence
  Synthetic polynucleotide
cggggttcgagtcggggttc

SEQ ID NO: 389 - DNA, 20 bp, Artificial Sequence
  Synthetic polynucleotide
tcggggttcgagtcggggtt

SEQ ID NO: 390 - DNA, 20 bp, Artificial Sequence
  Synthetic polynucleotide
tagtttcggggttcgagtcg

SEQ ID NO: 391 - DNA, 20 bp, Artificial Sequence
  Synthetic polynucleotide
ctagtttcggggttcgagtc

SEQ ID NO: 392 - DNA, 20 bp, Artificial Sequence
  Synthetic polynucleotide
tctagtttcggggttcgagt

SEQ ID NO: 393 - DNA, 20 bp, Artificial Sequence
  Synthetic polynucleotide
ccaggtccagtctagtttcg

SEQ ID NO: 394 - DNA, 20 bp, Artificial Sequence
  Synthetic polynucleotide
tccaggtccagtctagtttc

SEQ ID NO: 395 - DNA, 20 bp, Artificial Sequence
  Synthetic polynucleotide
gtccaggtccagtctagttt

SEQ ID NO: 396 - DNA, 20 bp, Artificial Sequence
  Synthetic polynucleotide
ctcgaaccccgaaactagac

SEQ ID NO: 397 - DNA, 20 bp, Artificial Sequence
  Synthetic polynucleotide
ccccgaaactagactggacc

SEQ ID NO: 398 - DNA, 20 bp, Artificial Sequence
  Synthetic polynucleotide
actagactggacctggactc

SEQ ID NO: 399 - DNA, 20 bp, Artificial Sequence
  Synthetic polynucleotide
taggtcctaggccagagtcc

SEQ ID NO: 400 - DNA, 20 bp, Artificial Sequence
  Synthetic polynucleotide
ctggacctggactctggcct

SEQ ID NO: 401 - DNA, 20 bp, Artificial Sequence
  Synthetic polynucleotide
ctagacccgagctaggtcct

SEQ ID NO: 402 - DNA, 20 bp, Artificial Sequence
  Synthetic polynucleotide
ctctggcctaggacctagct

SEQ ID NO: 403 - DNA, 20 bp, Artificial Sequence
  Synthetic polynucleotide
tctggcctaggacctagctc

SEQ ID NO: 404 - DNA, 20 bp, Artificial Sequence
  Synthetic polynucleotide
ctgaactctagacccgagct

SEQ ID NO: 405 - DNA, 20 bp, Artificial Sequence
  Synthetic polynucleotide
ctagctcgggtctagagttc

SEQ ID NO: 406 - DNA, 20 bp, Artificial Sequence
  Synthetic polynucleotide
tctagagttcaggtccagtc

SEQ ID NO: 407 - DNA, 20 bp, Artificial Sequence
  Synthetic polynucleotide
ctagagttcaggtccagtcc

SEQ ID NO: 408 - DNA, 20 bp, Artificial Sequence
  Synthetic polynucleotide
tagagttcaggtccagtccg

SEQ ID NO: 409 - DNA, 20 bp, Artificial Sequence
  Synthetic polynucleotide
cctgacctcggaccccggac

SEQ ID NO: 410 - DNA, 20 bp, Artificial Sequence
  Synthetic polynucleotide
cagatcctgacctcggaccc

SEQ ID NO: 411 - DNA, 20 bp, Artificial Sequence
  Synthetic polynucleotide
caggtccagtccggggtccg

SEQ ID NO: 412 - DNA, 20 bp, Artificial Sequence
  Synthetic polynucleotide
ccagtccggggtccgaggtc

SEQ ID NO: 413 - DNA, 20 bp, Artificial Sequence
  Synthetic polynucleotide
acgaacccagatcctgacct

SEQ ID NO: 414 - DNA, 20 bp, Artificial Sequence
  Synthetic polynucleotide
cggggtccgaggtcaggatc

SEQ ID NO: 415 - DNA, 20 bp, Artificial Sequence
  Synthetic polynucleotide
ggggtccgaggtcaggatct

SEQ ID NO: 416 - DNA, 20 bp, Artificial Sequence
  Synthetic polynucleotide
caggatctgggttcgtgttc

SEQ ID NO: 417 - DNA, 20 bp, Artificial Sequence
  Synthetic polynucleotide
aggatctgggttcgtgttct

SEQ ID NO: 418 - DNA, 20 bp, Artificial Sequence
  Synthetic polynucleotide
ggatctgggttcgtgttctg

SEQ ID NO: 419 - DNA, 20 bp, Artificial Sequence
  Synthetic polynucleotide
gggttcgtgttctggggttc

SEQ ID NO: 420 - DNA, 20 bp, Artificial Sequence
  Synthetic polynucleotide
tcgtgttctggggttcaggt

SEQ ID NO: 421 - DNA, 20 bp, Artificial Sequence
  Synthetic polynucleotide
cgtgttctggggttcaggtt

SEQ ID NO: 422 - DNA, 20 bp, Artificial Sequence
  Synthetic polynucleotide
ttctggggttcaggttgggt

SEQ ID NO: 423 - DNA, 20 bp, Artificial Sequence
  Synthetic polynucleotide
tctggggttcaggttgggtt

SEQ ID NO: 424 - DNA, 20 bp, Artificial Sequence
  Synthetic polynucleotide
ggttcaggttgggttgggtc

SEQ ID NO: 425 - DNA, 20 bp, Artificial Sequence
  Synthetic polynucleotide
gttgggttgggtctggagtc

SEQ ID NO: 426 - DNA, 20 bp, Artificial Sequence
  Synthetic polynucleotide
ttgggttgggtctggagtct

SEQ ID NO: 427 - DNA, 20 bp, Artificial Sequence
  Synthetic polynucleotide
tgggtctggagtctgggtct

SEQ ID NO: 428 - DNA, 20 bp, Artificial Sequence
  Synthetic polynucleotide
gggtctggagtctgggtcta

SEQ ID NO: 429 - DNA, 20 bp, Artificial Sequence
  Synthetic polynucleotide
tgggtctagggtccagattc

SEQ ID NO: 430 - DNA, 20 bp, Artificial Sequence
  Synthetic polynucleotide
cctgaacccgatcctgaatc

SEQ ID NO: 431 - DNA, 20 bp, Artificial Sequence
  Synthetic polynucleotide
ctagggtccagattcaggat

SEQ ID NO: 432 - DNA, 20 bp, Artificial Sequence
  Synthetic polynucleotide
tagggtccagattcaggatc

SEQ ID NO: 433 - DNA, 20 bp, Artificial Sequence
  Synthetic polynucleotide
ccagattcaggatcgggttc

SEQ ID NO: 434 - DNA, 20 bp, Artificial Sequence
  Synthetic polynucleotide
tcaggatcgggttcaggtta

SEQ ID NO: 435 - DNA, 20 bp, Artificial Sequence
  Synthetic polynucleotide
taaggtttgagtctgagtcc

SEQ ID NO: 436 - DNA, 20 bp, Artificial Sequence
  Synthetic polynucleotide
ttgagtctgagtccaggtat

SEQ ID NO: 437 - DNA, 20 bp, Artificial Sequence
  Synthetic polynucleotide
tcccgaccagaacctatacc

SEQ ID NO: 438 - DNA, 20 bp, Artificial Sequence
  Synthetic polynucleotide
ctgagtccaggtataggttc

SEQ ID NO: 439 - DNA, 20 bp, Artificial Sequence
  Synthetic polynucleotide
gtccaggtataggttctggt

SEQ ID NO: 440 - DNA, 20 bp, Artificial Sequence
  Synthetic polynucleotide
tccaggtataggttctggtc

SEQ ID NO: 441 - DNA, 20 bp, Artificial Sequence
  Synthetic polynucleotide
agttcgagagtttgaattca

SEQ ID NO: 442 - DNA, 20 bp, Artificial Sequence
  Synthetic polynucleotide
tttgaattcaaggtccaatt

SEQ ID NO: 443 - DNA, 20 bp, Artificial Sequence
  Synthetic polynucleotide
gaactcatccaactccaaat

SEQ ID NO: 444 - DNA, 20 bp, Artificial Sequence
  Synthetic polynucleotide
ttcaaggtccaatttggagt

SEQ ID NO: 445 - DNA, 20 bp, Artificial Sequence
  Synthetic polynucleotide
agttggatgagttcatgtca

SEQ ID NO: 446 - DNA, 20 bp, Artificial Sequence
  Synthetic polynucleotide
tttaaaattttaatagtgtt

SEQ ID NO: 447 - DNA, 20 bp, Artificial Sequence
  Synthetic polynucleotide
attcataatttttaaattag

SEQ ID NO: 448 - DNA, 20 bp, Artificial Sequence
  Synthetic polynucleotide
ttcataatttttaaattaga

SEQ ID NO: 449 - DNA, 20 bp, Artificial Sequence
  Synthetic polynucleotide
ttatttttatcttacttata

SEQ ID NO: 450 - DNA, 20 bp, Artificial Sequence
  Synthetic polynucleotide
attatttttatcttacttat

SEQ ID NO: 451 - DNA, 20 bp, Artificial Sequence
  Synthetic polynucleotide
tttactgtaccgaatattca

SEQ ID NO: 452 - DNA, 20 bp, Artificial Sequence
  Synthetic polynucleotide
ttgtagttaccgtgaatatt

SEQ ID NO: 453 - DNA, 20 bp, Artificial Sequence
  Synthetic polynucleotide
atattcggtacagtaaatta

SEQ ID NO: 454 - DNA, 20 bp, Artificial Sequence
  Synthetic polynucleotide
tcggtacagtaaattaagga

SEQ ID NO: 455 - DNA, 20 bp, Artificial Sequence
  Synthetic polynucleotide
atatataaaaatataaattg

SEQ ID NO: 456 - DNA, 20 bp, Artificial Sequence
  Synthetic polynucleotide
tatattattaatctagataa

SEQ ID NO: 457 - DNA, 20 bp, Artificial Sequence
  Synthetic polynucleotide
ataattatactatatattat

SEQ ID NO: 458 - DNA, 20 bp, Artificial Sequence
  Synthetic polynucleotide
ttataataattatacatgtt

SEQ ID NO: 459 - DNA, 20 bp, Artificial Sequence
  Synthetic polynucleotide
atgtttggcaatttcaattt

SEQ ID NO: 460 - DNA, 20 bp, Artificial Sequence
  Synthetic polynucleotide
ttggcaatttcaatttaggt

SEQ ID NO: 461 - DNA, 20 bp, Artificial Sequence
  Synthetic polynucleotide
aggttggtgactgatattcc

SEQ ID NO: 462 - DNA, 20 bp, Artificial Sequence
  Synthetic polynucleotide
aagcttggcccggtagttcc

SEQ ID NO: 463 - DNA, 20 bp, Artificial Sequence
  Synthetic polynucleotide
actgatattcctggaactac

SEQ ID NO: 464 - DNA, 20 bp, Artificial Sequence
  Synthetic polynucleotide
ctgatattcctggaactacc

SEQ ID NO: 465 - DNA, 20 bp, Artificial Sequence
  Synthetic polynucleotide
cggctcaccgaagcttggcc

SEQ ID NO: 466 - DNA, 20 bp, Artificial Sequence
  Synthetic polynucleotide
ggaactaccgggccaagctt

SEQ ID NO: 467 - DNA, 20 bp, Artificial Sequence
  Synthetic polynucleotide
atgaacggctcaccgaagct

SEQ ID NO: 468 - DNA, 20 bp, Artificial Sequence
  Synthetic polynucleotide
gtattattatgaagtatgaa

SEQ ID NO: 469 - DNA, 20 bp, Artificial Sequence
  Synthetic polynucleotide
acgctgtaaacaaaatagtg

SEQ ID NO: 470 - DNA, 20 bp, Artificial Sequence
  Synthetic polynucleotide
attaattgtttattatgtgt

SEQ ID NO: 471 - DNA, 20 bp, Artificial Sequence
  Synthetic polynucleotide
tttattatgtgtaggacaag

SEQ ID NO: 472 - DNA, 20 bp, Artificial Sequence
  Synthetic polynucleotide
attatgtgtaggacaagagg

SEQ ID NO: 473 - DNA, 20 bp, Artificial Sequence
  Synthetic polynucleotide
tggtgtgctacgagaacccg

SEQ ID NO: 474 - DNA, 20 bp, Artificial Sequence
  Synthetic polynucleotide
gaatccccaccgtcggccgc

SEQ ID NO: 475 - DNA, 20 bp, Artificial Sequence
  Synthetic polynucleotide
tgaatccccaccgtcggccg

SEQ ID NO: 476 - DNA, 20 bp, Artificial Sequence
  Synthetic polynucleotide
ctacgagaacccgcggccga

SEQ ID NO: 477 - DNA, 20 bp, Artificial Sequence
  Synthetic polynucleotide
cgagaacccgcggccgacgg

SEQ ID NO: 478 - DNA, 20 bp, Artificial Sequence
  Synthetic polynucleotide
gagaacccgcggccgacggt

SEQ ID NO: 479 - DNA, 20 bp, Artificial Sequence
  Synthetic polynucleotide
agaacccgcggccgacggtg

SEQ ID NO: 480 - DNA, 20 bp, Artificial Sequence
  Synthetic polynucleotide
taccgatgaatccccaccgt

SEQ ID NO: 481 - DNA, 20 bp, Artificial Sequence
  Synthetic polynucleotide
ggccgacggtggggattcat

SEQ ID NO: 482 - DNA, 20 bp, Artificial Sequence
  Synthetic polynucleotide
ggtatgtatttgtgttgttc

SEQ ID NO: 483 - DNA, 20 bp, Artificial Sequence
  Synthetic polynucleotide
atttgtgttgttccggcaat

SEQ ID NO: 484 - DNA, 20 bp, Artificial Sequence
  Synthetic polynucleotide
tttgtgttgttccggcaatt

SEQ ID NO: 485 - DNA, 20 bp, Artificial Sequence
  Synthetic polynucleotide
ccgtttgccttcccaattgc

SEQ ID NO: 486 - DNA, 20 bp, Artificial Sequence
  Synthetic polynucleotide
tgttgttccggcaattggga

SEQ ID NO: 487 - DNA, 20 bp, Artificial Sequence
  Synthetic polynucleotide
ccggcaattgggaaggcaaa

SEQ ID NO: 488 - DNA, 20 bp, Artificial Sequence
  Synthetic polynucleotide
aaggcaaacggtgttcgcgc

SEQ ID NO: 489 - DNA, 20 bp, Artificial Sequence
  Synthetic polynucleotide
aggcaaacggtgttcgcgcc

SEQ ID NO: 490 - DNA, 20 bp, Artificial Sequence
  Synthetic polynucleotide
ggcaaacggtgttcgcgccg

SEQ ID NO: 491 - DNA, 20 bp, Artificial Sequence
  Synthetic polynucleotide
aaacggtgttcgcgccgggg

SEQ ID NO: 492 - DNA, 20 bp, Artificial Sequence
  Synthetic polynucleotide
ttgaagttctgacgccaccc

SEQ ID NO: 493 - DNA, 20 bp, Artificial Sequence
  Synthetic polynucleotide
ataaagctcagcaaagtctt

SEQ ID NO: 494 - DNA, 20 bp, Artificial Sequence
  Synthetic polynucleotide
tttgctgagctttataacct

SEQ ID NO: 495 - DNA, 20 bp, Artificial Sequence
  Synthetic polynucleotide
gggcagcaacaggcaaacca

SEQ ID NO: 496 - DNA, 20 bp, Artificial Sequence
  Synthetic polynucleotide
ttgtaataaagggcagcaac

SEQ ID NO: 497 - DNA, 20 bp, Artificial Sequence
  Synthetic polynucleotide
cctttggcagttgtaataaa

SEQ ID NO: 498 - DNA, 20 bp, Artificial Sequence
  Synthetic polynucleotide
ccctttggcagttgtaataa

SEQ ID NO: 499 - DNA, 20 bp, Artificial Sequence
  Synthetic polynucleotide
ccctttattacaactgccaa

SEQ ID NO: 500 - DNA, 20 bp, Artificial Sequence
  Synthetic polynucleotide
cctttattacaactgccaaa

SEQ ID NO: 501 - DNA, 20 bp, Artificial Sequence
  Synthetic polynucleotide
ccccagatcctgtctccctt

SEQ ID NO: 502 - DNA, 20 bp, Artificial Sequence
  Synthetic polynucleotide
tacaactgccaaagggagac

SEQ ID NO: 503 - DNA, 20 bp, Artificial Sequence
  Synthetic polynucleotide
tgccaaagggagacaggatc

SEQ ID NO: 504 - DNA, 20 bp, Artificial Sequence
  Synthetic polynucleotide
gccaaagggagacaggatct

SEQ ID NO: 505 - DNA, 20 bp, Artificial Sequence
  Synthetic polynucleotide
ccaaagggagacaggatctg

SEQ ID NO: 506 - DNA, 20 bp, Artificial Sequence
  Synthetic polynucleotide
caaagggagacaggatctgg

SEQ ID NO: 507 - DNA, 20 bp, Artificial Sequence
  Synthetic polynucleotide
gggagacaggatctggggga

SEQ ID NO: 508 - DNA, 20 bp, Artificial Sequence
  Synthetic polynucleotide
agacaggatctgggggaagg

SEQ ID NO: 509 - DNA, 20 bp, Artificial Sequence
  Synthetic polynucleotide
atatatatatgtctatgtgg

SEQ ID NO: 510 - DNA, 20 bp, Artificial Sequence
  Synthetic polynucleotide
tatatatatatatgtctatg

SEQ ID NO: 511 - DNA, 20 bp, Artificial Sequence
  Synthetic polynucleotide
gattcttaatgatgatatca

SEQ ID NO: 512 - DNA, 20 bp, Artificial Sequence
  Synthetic polynucleotide
aaagcttattatattaataa

SEQ ID NO: 513 - DNA, 20 bp, Artificial Sequence
  Synthetic polynucleotide
aagatgaagaagaagaaaac

SEQ ID NO: 514 - DNA, 20 bp, Artificial Sequence
  Synthetic polynucleotide
attattgtacttaattcagc

SEQ ID NO: 515 - DNA, 20 bp, Artificial Sequence
  Synthetic polynucleotide
tacataatataatattagca

SEQ ID NO: 516 - DNA, 20 bp, Artificial Sequence
  Synthetic polynucleotide
atgagatacacacatatata

SEQ ID NO: 517 - DNA, 20 bp, Artificial Sequence
  Synthetic polynucleotide
tataattaattaagaatata

SEQ ID NO: 518 - DNA, 20 bp, Artificial Sequence
  Synthetic polynucleotide
aagaatatatggctagagat

SEQ ID NO: 519 - DNA, 20 bp, Artificial Sequence
  Synthetic polynucleotide
agaatatatggctagagata

SEQ ID NO: 520 - DNA, 20 bp, Artificial Sequence
  Synthetic polynucleotide
tagagatagggaccctcttg

SEQ ID NO: 521 - DNA, 20 bp, Artificial Sequence
  Synthetic polynucleotide
ttactctaccaaccacaaga

SEQ ID NO: 522 - DNA, 20 bp, Artificial Sequence
  Synthetic polynucleotide
attactctaccaaccacaag

SEQ ID NO: 523 - DNA, 20 bp, Artificial Sequence
  Synthetic polynucleotide
gatagggaccctcttgtggt

SEQ ID NO: 524 - DNA, 20 bp, Artificial Sequence
  Synthetic polynucleotide
cttgtggttggtagagtaat

SEQ ID NO: 525 - DNA, 20 bp, Artificial Sequence
  Synthetic polynucleotide
tagagtaataggagatgttt

SEQ ID NO: 526 - DNA, 20 bp, Artificial Sequence
  Synthetic polynucleotide
atgttttggatccttttaca

SEQ ID NO: 527 - DNA, 20 bp, Artificial Sequence
  Synthetic polynucleotide
agagagactgaccttgtaaa

SEQ ID NO: 528 - DNA, 20 bp, Artificial Sequence
  Synthetic polynucleotide
gtctctcttagagtgagtta

SEQ ID NO: 529 - DNA, 20 bp, Artificial Sequence
  Synthetic polynucleotide
gagtgagttatggtaatgag

SEQ ID NO: 530 - DNA, 20 bp, Artificial Sequence
  Synthetic polynucleotide
ggtaatgagaggtcaacaga

SEQ ID NO: 531 - DNA, 20 bp, Artificial Sequence
  Synthetic polynucleotide
ggttggttaacaatttggga

SEQ ID NO: 532 - DNA, 20 bp, Artificial Sequence
  Synthetic polynucleotide
acgaggttggttaacaattt

SEQ ID NO: 533 - DNA, 20 bp, Artificial Sequence
  Synthetic polynucleotide
cacgaggttggttaacaatt

SEQ ID NO: 534 - DNA, 20 bp, Artificial Sequence
  Synthetic polynucleotide
caccaatatcaacacgaggt

SEQ ID NO: 535 - DNA, 20 bp, Artificial Sequence
  Synthetic polynucleotide
tcaccaccaatatcaacacg

SEQ ID NO: 536 - DNA, 20 bp, Artificial Sequence
  Synthetic polynucleotide
aaccaacctcgtgttgatat

SEQ ID NO: 537 - DNA, 20 bp, Artificial Sequence
  Synthetic polynucleotide
caacctcgtgttgatattgg

SEQ ID NO: 538 - DNA, 20 bp, Artificial Sequence
  Synthetic polynucleotide
atattggtggtgatgaccta

SEQ ID NO: 539 - DNA, 20 bp, Artificial Sequence
  Synthetic polynucleotide
ccaaagtgtagaaggtcctt

SEQ ID NO: 540 - DNA, 20 bp, Artificial Sequence
  Synthetic polynucleotide
ttaatttaccaaagtgtaga

SEQ ID NO: 541 - DNA, 20 bp, Artificial Sequence
  Synthetic polynucleotide
cctaaggaccttctacactt

SEQ ID NO: 542 - DNA, 20 bp, Artificial Sequence
  Synthetic polynucleotide
tgaaatcattatgaatattg

SEQ ID NO: 543 - DNA, 20 bp, Artificial Sequence
  Synthetic polynucleotide
tcatatattgaaattattac

SEQ ID NO: 544 - DNA, 20 bp, Artificial Sequence
  Synthetic polynucleotide
attgaaattattacaggtca

SEQ ID NO: 545 - DNA, 20 bp, Artificial Sequence
  Synthetic polynucleotide
gaaattattacaggtcatgg

SEQ ID NO: 546 - DNA, 20 bp, Artificial Sequence
  Synthetic polynucleotide
attacaggtcatggtggatc

SEQ ID NO: 547 - DNA, 20 bp, Artificial Sequence
  Synthetic polynucleotide
ttgctagggctaggagcatc

SEQ ID NO: 548 - DNA, 20 bp, Artificial Sequence
  Synthetic polynucleotide
agattggggttgctagggct

SEQ ID NO: 549 - DNA, 20 bp, Artificial Sequence
  Synthetic polynucleotide
cccttagattggggttgcta

SEQ ID NO: 550 - DNA, 20 bp, Artificial Sequence
  Synthetic polynucleotide
tcccttagattggggttgct

SEQ ID NO: 551 - DNA, 20 bp, Artificial Sequence
  Synthetic polynucleotide
gcaaatactcccttagattg

SEQ ID NO: 552 - DNA, 20 bp, Artificial Sequence
  Synthetic polynucleotide
gccctagcaaccccaatcta

SEQ ID NO: 553 - DNA, 20 bp, Artificial Sequence
  Synthetic polynucleotide
tgcaaatactcccttagatt

SEQ ID NO: 554 - DNA, 20 bp, Artificial Sequence
  Synthetic polynucleotide
ccctagcaaccccaatctaa

SEQ ID NO: 555 - DNA, 20 bp, Artificial Sequence
  Synthetic polynucleotide
atgcaaatactcccttagat

SEQ ID NO: 556 - DNA, 20 bp, Artificial Sequence
  Synthetic polynucleotide
atctaagggagtatttgcat

SEQ ID NO: 557 - DNA, 20 bp, Artificial Sequence
  Synthetic polynucleotide
tattattattataatagatg

SEQ ID NO: 558 - DNA, 20 bp, Artificial Sequence
  Synthetic polynucleotide
attattattataatagatga

SEQ ID NO: 559 - DNA, 20 bp, Artificial Sequence
  Synthetic polynucleotide
tttaattttgtataaaaatt

SEQ ID NO: 560 - DNA, 20 bp, Artificial Sequence
  Synthetic polynucleotide
ttcatgcacacaacacatgt

SEQ ID NO: 561 - DNA, 20 bp, Artificial Sequence
  Synthetic polynucleotide
caaaagataaagacatattt

SEQ ID NO: 562 - DNA, 20 bp, Artificial Sequence
  Synthetic polynucleotide
caaaatatgtctttatcttt

SEQ ID NO: 563 - DNA, 20 bp, Artificial Sequence
  Synthetic polynucleotide
cattttataaagatgttagt

SEQ ID NO: 564 - DNA, 20 bp, Artificial Sequence
  Synthetic polynucleotide
attttataaagatgttagtt

SEQ ID NO: 565 - DNA, 20 bp, Artificial Sequence
  Synthetic polynucleotide
ttttagtgtcagttttgaat

SEQ ID NO: 566 - DNA, 20 bp, Artificial Sequence
  Synthetic polynucleotide
acaaaattctgtaattatta

SEQ ID NO: 567 - DNA, 20 bp, Artificial Sequence
  Synthetic polynucleotide
tacaaaattctgtaattatt

SEQ ID NO: 568 - DNA, 20 bp, Artificial Sequence
  Synthetic polynucleotide
acaaattaaaacaagcttta

SEQ ID NO: 569 - DNA, 20 bp, Artificial Sequence
  Synthetic polynucleotide
aacaaattaaaacaagcttt

SEQ ID NO: 570 - DNA, 20 bp, Artificial Sequence
  Synthetic polynucleotide
tttaatttgttaaagtgact

SEQ ID NO: 571 - DNA, 20 bp, Artificial Sequence
  Synthetic polynucleotide
aaacatgtaaaagaatttaa

SEQ ID NO: 572 - DNA, 20 bp, Artificial Sequence
  Synthetic polynucleotide
taaacatgtaaaagaattta

SEQ ID NO: 573 - DNA, 20 bp, Artificial Sequence
  Synthetic polynucleotide
tagctagcatatatggattg

SEQ ID NO: 574 - DNA, 20 bp, Artificial Sequence
  Synthetic polynucleotide
atttatatagctagcatata

SEQ ID NO: 575 - DNA, 20 bp, Artificial Sequence
  Synthetic polynucleotide
tagctatataaatataaata

SEQ ID NO: 576 - DNA, 20 bp, Artificial Sequence
  Synthetic polynucleotide
atataaatataaatatggaa

SEQ ID NO: 577 - DNA, 20 bp, Artificial Sequence
  Synthetic polynucleotide
ataaatatggaaaggatata

SEQ ID NO: 578 - DNA, 20 bp, Artificial Sequence
  Synthetic polynucleotide
taaatatggaaaggatatat

SEQ ID NO: 579 - DNA, 20 bp, Artificial Sequence
  Synthetic polynucleotide
aaagctgatgagaaagaatg

SEQ ID NO: 580 - DNA, 20 bp, Artificial Sequence
  Synthetic polynucleotide
tgatgagaaagaatgtggtt

SEQ ID NO: 581 - DNA, 20 bp, Artificial Sequence
  Synthetic polynucleotide
gatgagaaagaatgtggttt

SEQ ID NO: 582 - DNA, 20 bp, Artificial Sequence
  Synthetic polynucleotide
atgagaaagaatgtggtttg

SEQ ID NO: 583 - DNA, 20 bp, Artificial Sequence
  Synthetic polynucleotide
ggatgaattttgaatgatga

SEQ ID NO: 584 - DNA, 20 bp, Artificial Sequence
  Synthetic polynucleotide
gatgaattttgaatgatgaa

SEQ ID NO: 585 - DNA, 20 bp, Artificial Sequence
  Synthetic polynucleotide
ttttgaatgatgaagggatg

SEQ ID NO: 586 - DNA, 20 bp, Artificial Sequence
  Synthetic polynucleotide
aagggatgaggctgtgtgtg

SEQ ID NO: 587 - DNA, 20 bp, Artificial Sequence
  Synthetic polynucleotide
gggacatgctatagctagca

SEQ ID NO: 588 - DNA, 20 bp, Artificial Sequence
  Synthetic polynucleotide
ggggacatgctatagctagc

SEQ ID NO: 589 - DNA, 20 bp, Artificial Sequence
  Synthetic polynucleotide
attttaatgggggacaaaag

SEQ ID NO: 590 - DNA, 20 bp, Artificial Sequence
  Synthetic polynucleotide
cattttaatgggggacaaaa

SEQ ID NO: 591 - DNA, 20 bp, Artificial Sequence
  Synthetic polynucleotide
ccattttaatgggggacaaa

SEQ ID NO: 592 - DNA, 20 bp, Artificial Sequence
  Synthetic polynucleotide
tgaggtggccattttaatgg

SEQ ID NO: 593 - DNA, 20 bp, Artificial Sequence
  Synthetic polynucleotide
gtgaggtggccattttaatg

SEQ ID NO: 594 - DNA, 20 bp, Artificial Sequence
  Synthetic polynucleotide
tgtgaggtggccattttaat

SEQ ID NO: 595 - DNA, 20 bp, Artificial Sequence
  Synthetic polynucleotide
ccttttgtcccccattaaaa

SEQ ID NO: 596 - DNA, 20 bp, Artificial Sequence
  Synthetic polynucleotide
gtgtgaggtggccattttaa

SEQ ID NO: 597 - DNA, 20 bp, Artificial Sequence
  Synthetic polynucleotide
aaaaccttcttagtgtgagg

SEQ ID NO: 598 - DNA, 20 bp, Artificial Sequence
  Synthetic polynucleotide
gtgaaaaccttcttagtgtg

SEQ ID NO: 599 - DNA, 20 bp, Artificial Sequence
  Synthetic polynucleotide
atggccacctcacactaaga

SEQ ID NO: 600 - DNA, 20 bp, Artificial Sequence
  Synthetic polynucleotide
atatatacatacacatgtat

SEQ ID NO: 601 - DNA, 20 bp, Artificial Sequence
  Synthetic polynucleotide
tatatacatacacatgtata

SEQ ID NO: 602 - DNA, 20 bp, Artificial Sequence
  Synthetic polynucleotide
ctatgttcgaattcaaattc

SEQ ID NO: 603 - DNA, 20 bp, Artificial Sequence
  Synthetic polynucleotide
tctatgttcgaattcaaatt

SEQ ID NO: 604 - DNA, 20 bp, Artificial Sequence
  Synthetic polynucleotide
ttcgaacatagactcagatt

SEQ ID NO: 605 - DNA, 20 bp, Artificial Sequence
  Synthetic polynucleotide
cagggttcagggtcgaatta

SEQ ID NO: 606 - DNA, 20 bp, Artificial Sequence
  Synthetic polynucleotide
tcagggttcagggtcgaatt

SEQ ID NO: 607 - DNA, 20 bp, Artificial Sequence
  Synthetic polynucleotide
agttcgtgtttcagggttca

SEQ ID NO: 608 - DNA, 20 bp, Artificial Sequence
  Synthetic polynucleotide
aagttcgtgtttcagggttc

SEQ ID NO: 609 - DNA, 20 bp, Artificial Sequence
  Synthetic polynucleotide
gagtctaagttcgtgtttca

SEQ ID NO: 610 - DNA, 20 bp, Artificial Sequence
  Synthetic polynucleotide
tgagtctaagttcgtgtttc

SEQ ID NO: 611 - DNA, 20 bp, Artificial Sequence
  Synthetic polynucleotide
cacgaacttagactcagacc

SEQ ID NO: 612 - DNA, 20 bp, Artificial Sequence
  Synthetic polynucleotide
acttagactcagacctggac

SEQ ID NO: 613 - DNA, 20 bp, Artificial Sequence
  Synthetic polynucleotide
tttgggtcaaggtccagtcc

SEQ ID NO: 614 - DNA, 20 bp, Artificial Sequence
  Synthetic polynucleotide
tcgggtcggggtttgggtca

SEQ ID NO: 615 - DNA, 20 bp, Artificial Sequence
  Synthetic polynucleotide
cggggttcgggtcggggttt

SEQ ID NO: 616 - DNA, 20 bp, Artificial Sequence
  Synthetic polynucleotide
tcggggttcgggtcggggtt

SEQ ID NO: 617 - DNA, 20 bp, Artificial Sequence
  Synthetic polynucleotide
tcgagtcggggttcgggtcg

SEQ ID NO: 618 - DNA, 20 bp, Artificial Sequence
  Synthetic polynucleotide
ttcgagtcggggttcgggtc

SEQ ID NO: 619 - DNA, 20 bp, Artificial Sequence
  Synthetic polynucleotide
gttcgagtcggggttcgggt

SEQ ID NO: 620 - DNA, 20 bp, Artificial Sequence
  Synthetic polynucleotide
cggggttcgagtcggggttc

SEQ ID NO: 621 - DNA, 20 bp, Artificial Sequence
  Synthetic polynucleotide
tcggggttcgagtcggggtt

SEQ ID NO: 622 - DNA, 20 bp, Artificial Sequence
  Synthetic polynucleotide
tagtttcggggttcgagtcg

SEQ ID NO: 623 - DNA, 20 bp, Artificial Sequence
  Synthetic polynucleotide
ctagtttcggggttcgagtc

SEQ ID NO: 624 - DNA, 20 bp, Artificial Sequence
  Synthetic polynucleotide
tctagtttcggggttcgagt

SEQ ID NO: 625 - DNA, 20 bp, Artificial Sequence
  Synthetic polynucleotide
ccaggtccagtctagtttcg

SEQ ID NO: 626 - DNA, 20 bp, Artificial Sequence
  Synthetic polynucleotide
tccaggtccagtctagtttc

SEQ ID NO: 627 - DNA, 20 bp, Artificial Sequence
  Synthetic polynucleotide
gtccaggtccagtctagttt

SEQ ID NO: 628 - DNA, 20 bp, Artificial Sequence
  Synthetic polynucleotide
ctcgaaccccgaaactagac

SEQ ID NO: 629 - DNA, 20 bp, Artificial Sequence
  Synthetic polynucleotide
ccccgaaactagactggacc

SEQ ID NO: 630 - DNA, 20 bp, Artificial Sequence
  Synthetic polynucleotide
actagactggacctggactc

SEQ ID NO: 631 - DNA, 20 bp, Artificial Sequence
  Synthetic polynucleotide
taggtcctaggccagagtcc

SEQ ID NO: 632 - DNA, 20 bp, Artificial Sequence
  Synthetic polynucleotide
ctggacctggactctggcct

SEQ ID NO: 633 - DNA, 20 bp, Artificial Sequence
  Synthetic polynucleotide
ctagacccgagctaggtcct

SEQ ID NO: 634 - DNA, 20 bp, Artificial Sequence
  Synthetic polynucleotide
ctctggcctaggacctagct

SEQ ID NO: 635 - DNA, 20 bp, Artificial Sequence
  Synthetic polynucleotide
tctggcctaggacctagctc

SEQ ID NO: 636 - DNA, 20 bp, Artificial Sequence
  Synthetic polynucleotide
gcctgacctagacccgagct

SEQ ID NO: 637 - DNA, 20 bp, Artificial Sequence
  Synthetic polynucleotide
ctaggacctagctcgggtct

SEQ ID NO: 638 - DNA, 20 bp, Artificial Sequence
  Synthetic polynucleotide
acctagctcgggtctaggtc

SEQ ID NO: 639 - DNA, 20 bp, Artificial Sequence
  Synthetic polynucleotide
tcgggtctaggtcaggcgtc

SEQ ID NO: 640 - DNA, 20 bp, Artificial Sequence
  Synthetic polynucleotide
cgggtctaggtcaggcgtcc

SEQ ID NO: 641 - DNA, 20 bp, Artificial Sequence
  Synthetic polynucleotide
aggtcaggcgtccgggtccg

SEQ ID NO: 642 - DNA, 20 bp, Artificial Sequence
  Synthetic polynucleotide
cccagatctgacctcggacc

SEQ ID NO: 643 - DNA, 20 bp, Artificial Sequence
  Synthetic polynucleotide
cacgaacccagatctgacct

SEQ ID NO: 644 - DNA, 20 bp, Artificial Sequence
  Synthetic polynucleotide
tccgggtccgaggtcagatc

SEQ ID NO: 645 - DNA, 20 bp, Artificial Sequence
  Synthetic polynucleotide
ccgggtccgaggtcagatct

SEQ ID NO: 646 - DNA, 20 bp, Artificial Sequence
  Synthetic polynucleotide
tcagatctgggttcgtgttc

SEQ ID NO: 647 - DNA, 20 bp, Artificial Sequence
  Synthetic polynucleotide
cagatctgggttcgtgttct

SEQ ID NO: 648 - DNA, 20 bp, Artificial Sequence
  Synthetic polynucleotide
agatctgggttcgtgttctg

SEQ ID NO: 649 - DNA, 20 bp, Artificial Sequence
  Synthetic polynucleotide
gggttcgtgttctggggttc

SEQ ID NO: 650 - DNA, 20 bp, Artificial Sequence
  Synthetic polynucleotide
tcgtgttctggggttcaggt

SEQ ID NO: 651 - DNA, 20 bp, Artificial Sequence
  Synthetic polynucleotide
cgtgttctggggttcaggtt

SEQ ID NO: 652 - DNA, 20 bp, Artificial Sequence
  Synthetic polynucleotide
ttctggggttcaggttgggt

SEQ ID NO: 653 - DNA, 20 bp, Artificial Sequence
  Synthetic polynucleotide
tctggggttcaggttgggtt

SEQ ID NO: 654 - DNA, 20 bp, Artificial Sequence
  Synthetic polynucleotide
ggttcaggttgggttgggtc

SEQ ID NO: 655 - DNA, 20 bp, Artificial Sequence
  Synthetic polynucleotide
gttgggttgggtctggagtc

SEQ ID NO: 656 - DNA, 20 bp, Artificial Sequence
  Synthetic polynucleotide
ttgggttgggtctggagtct

SEQ ID NO: 657 - DNA, 20 bp, Artificial Sequence
  Synthetic polynucleotide
tgggtctggagtctgggtct

SEQ ID NO: 658 - DNA, 20 bp, Artificial Sequence
  Synthetic polynucleotide
gggtctggagtctgggtcta

SEQ ID NO: 659 - DNA, 20 bp, Artificial Sequence
  Synthetic polynucleotide
tgggtctagggtccagattc

SEQ ID NO: 660 - DNA, 20 bp, Artificial Sequence
  Synthetic polynucleotide
cctgaacccgatcctgaatc

SEQ ID NO: 661 - DNA, 20 bp, Artificial Sequence
  Synthetic polynucleotide
ctagggtccagattcaggat

SEQ ID NO: 662 - DNA, 20 bp, Artificial Sequence
  Synthetic polynucleotide
tagggtccagattcaggatc

SEQ ID NO: 663 - DNA, 20 bp, Artificial Sequence
  Synthetic polynucleotide
ccagattcaggatcgggttc

SEQ ID NO: 664 - DNA, 20 bp, Artificial Sequence
  Synthetic polynucleotide
tcaggatcgggttcaggtta

SEQ ID NO: 665 - DNA, 20 bp, Artificial Sequence
  Synthetic polynucleotide
taaggtttgagtctgagtcc

SEQ ID NO: 666 - DNA, 20 bp, Artificial Sequence
  Synthetic polynucleotide
ttgagtctgagtccaggtat

SEQ ID NO: 667 - DNA, 20 bp, Artificial Sequence
  Synthetic polynucleotide
tcccgaccagaacctatacc

SEQ ID NO: 668 - DNA, 20 bp, Artificial Sequence
  Synthetic polynucleotide
ctgagtccaggtataggttc

SEQ ID NO: 669 - DNA, 20 bp, Artificial Sequence
  Synthetic polynucleotide
gtccaggtataggttctggt

SEQ ID NO: 670 - DNA, 20 bp, Artificial Sequence
  Synthetic polynucleotide
tccaggtataggttctggtc

SEQ ID NO: 671 - DNA, 20 bp, Artificial Sequence
  Synthetic polynucleotide
gagttcagagtttgaattca

SEQ ID NO: 672 - DNA, 20 bp, Artificial Sequence
  Synthetic polynucleotide
tttgaattcaaggtccaatt

SEQ ID NO: 673 - DNA, 20 bp, Artificial Sequence
  Synthetic polynucleotide
gaactcatccaactccaaat

SEQ ID NO: 674 - DNA, 20 bp, Artificial Sequence
  Synthetic polynucleotide
ttcaaggtccaatttggagt

SEQ ID NO: 675 - DNA, 20 bp, Artificial Sequence
  Synthetic polynucleotide
agttggatgagttcatgtca

SEQ ID NO: 676 - DNA, 20 bp, Artificial Sequence
  Synthetic polynucleotide
tttaaaattttaatagtgtt

SEQ ID NO: 677 - DNA, 20 bp, Artificial Sequence
  Synthetic polynucleotide
ttcataatttttaaaattag

SEQ ID NO: 678 - DNA, 20 bp, Artificial Sequence
  Synthetic polynucleotide
tcataatttttaaaattaga

SEQ ID NO: 679 - DNA, 20 bp, Artificial Sequence
  Synthetic polynucleotide
tatttttatctttacttata

SEQ ID NO: 680 - DNA, 20 bp, Artificial Sequence
  Synthetic polynucleotide
ttatttttatctttacttat

SEQ ID NO: 681 - DNA, 20 bp, Artificial Sequence
  Synthetic polynucleotide
tttactgtaccgaatattca

SEQ ID NO: 682 - DNA, 20 bp, Artificial Sequence
  Synthetic polynucleotide
ttgtagttaccgtgaatatt

SEQ ID NO: 683 - DNA, 20 bp, Artificial Sequence
  Synthetic polynucleotide
atattcggtacagtaaatta

SEQ ID NO: 684 - DNA, 20 bp, Artificial Sequence
  Synthetic polynucleotide
tcggtacagtaaattaagga

SEQ ID NO: 685 - DNA, 20 bp, Artificial Sequence
  Synthetic polynucleotide
atatataaaaatataaattg

SEQ ID NO: 686 - DNA, 20 bp, Artificial Sequence
  Synthetic polynucleotide
tatattattaatctagataa

SEQ ID NO: 687 - DNA, 20 bp, Artificial Sequence
  Synthetic polynucleotide
ttataattatactaatatat

SEQ ID NO: 688 - DNA, 20 bp, Artificial Sequence
  Synthetic polynucleotide
ttataataattatacatgtt

SEQ ID NO: 689 - DNA, 20 bp, Artificial Sequence
  Synthetic polynucleotide
tgtttggcaatttcaatttt

SEQ ID NO: 690 - DNA, 20 bp, Artificial Sequence
  Synthetic polynucleotide
tggcaatttcaattttaggt

SEQ ID NO: 691 - DNA, 20 bp, Artificial Sequence
  Synthetic polynucleotide
aggttggtgactgatattcc

SEQ ID NO: 692 - DNA, 20 bp, Artificial Sequence
  Synthetic polynucleotide
aagcttggcccggtagttcc

SEQ ID NO: 693 - DNA, 20 bp, Artificial Sequence
  Synthetic polynucleotide
actgatattcctggaactac

SEQ ID NO: 694 - DNA, 20 bp, Artificial Sequence
  Synthetic polynucleotide
ctgatattcctggaactacc

SEQ ID NO: 695 - DNA, 20 bp, Artificial Sequence
  Synthetic polynucleotide
cggctcaccgaagcttggcc

SEQ ID NO: 696 - DNA, 20 bp, Artificial Sequence
  Synthetic polynucleotide
ggaactaccgggccaagctt

SEQ ID NO: 697 - DNA, 20 bp, Artificial Sequence
  Synthetic polynucleotide
atgaacggctcaccgaagct

SEQ ID NO: 698 - DNA, 20 bp, Artificial Sequence
  Synthetic polynucleotide
gtattattatgaagtatgaa

SEQ ID NO: 699 - DNA, 20 bp, Artificial Sequence
  Synthetic polynucleotide
acgctgtaaacaaaatagtg

SEQ ID NO: 700 - DNA, 20 bp, Artificial Sequence
  Synthetic polynucleotide
attaattgtttattatgtgt

SEQ ID NO: 701 - DNA, 20 bp, Artificial Sequence
  Synthetic polynucleotide
tttattatgtgtaggacaag

SEQ ID NO: 702 - DNA, 20 bp, Artificial Sequence
  Synthetic polynucleotide
attatgtgtaggacaagagg

SEQ ID NO: 703 - DNA, 20 bp, Artificial Sequence
  Synthetic polynucleotide
tggtgtgctacgagaacccg

SEQ ID NO: 704 - DNA, 20 bp, Artificial Sequence
  Synthetic polynucleotide
gtgctacgagaacccgcggc

SEQ ID NO: 705 - DNA, 20 bp, Artificial Sequence
  Synthetic polynucleotide
tgctacgagaacccgcggcc

SEQ ID NO: 706 - DNA, 20 bp, Artificial Sequence
  Synthetic polynucleotide
atgaatccccacccggccgc

SEQ ID NO: 707 - DNA, 20 bp, Artificial Sequence
  Synthetic polynucleotide
gatgaatccccacccggccg

SEQ ID NO: 708 - DNA, 20 bp, Artificial Sequence
  Synthetic polynucleotide
tacgagaacccgcggccggg

SEQ ID NO: 709 - DNA, 20 bp, Artificial Sequence
  Synthetic polynucleotide
acgagaacccgcggccgggt

SEQ ID NO: 710 - DNA, 20 bp, Artificial Sequence
  Synthetic polynucleotide
cgagaacccgcggccgggtg

SEQ ID NO: 711 - DNA, 20 bp, Artificial Sequence
  Synthetic polynucleotide
cataccgatgaatccccacc

SEQ ID NO: 712 - DNA, 20 bp, Artificial Sequence
  Synthetic polynucleotide
gcggccgggtggggattcat

SEQ ID NO: 713 - DNA, 20 bp, Artificial Sequence
  Synthetic polynucleotide
ggtatgtatttgtgttgttc

SEQ ID NO: 714 - DNA, 20 bp, Artificial Sequence
  Synthetic polynucleotide
atttgtgttgttccggcaat

SEQ ID NO: 715 - DNA, 20 bp, Artificial Sequence
  Synthetic polynucleotide
tttgtgttgttccggcaatt

SEQ ID NO: 716 - DNA, 20 bp, Artificial Sequence
  Synthetic polynucleotide
ccgtttgccttcccaattgc

SEQ ID NO: 717 - DNA, 20 bp, Artificial Sequence
  Synthetic polynucleotide
tgttgttccggcaattggga

SEQ ID NO: 718 - DNA, 20 bp, Artificial Sequence
  Synthetic polynucleotide
ccggcaattgggaaggcaaa

SEQ ID NO: 719 - DNA, 20 bp, Artificial Sequence
  Synthetic polynucleotide
aaggcaaacggtgttcgcgc

SEQ ID NO: 720 - DNA, 20 bp, Artificial Sequence
  Synthetic polynucleotide
aggcaaacggtgttcgcgcc

SEQ ID NO: 721 - DNA, 20 bp, Artificial Sequence
  Synthetic polynucleotide
ggcaaacggtgttcgcgccg

SEQ ID NO: 722 - DNA, 20 bp, Artificial Sequence
  Synthetic polynucleotide
aaacggtgttcgcgccgggg

SEQ ID NO: 723 - DNA, 20 bp, Artificial Sequence
  Synthetic polynucleotide
ttgaagttctgacgccaccc

SEQ ID NO: 724 - DNA, 20 bp, Artificial Sequence
  Synthetic polynucleotide
ataaagctcagcaaagtctt

SEQ ID NO: 725 - DNA, 20 bp, Artificial Sequence
  Synthetic polynucleotide
tttgctgagctttataacct

SEQ ID NO: 726 - DNA, 20 bp, Artificial Sequence
  Synthetic polynucleotide
gggcagcaacaggcaaacca

SEQ ID NO: 727 - DNA, 20 bp, Artificial Sequence
  Synthetic polynucleotide
ttgtaataaagggcagcaac

SEQ ID NO: 728 - DNA, 20 bp, Artificial Sequence
  Synthetic polynucleotide
cctttggcagttgtaataaa

SEQ ID NO: 729 - DNA, 20 bp, Artificial Sequence
  Synthetic polynucleotide
ccctttggcagttgtaataa

SEQ ID NO: 730 - DNA, 20 bp, Artificial Sequence
  Synthetic polynucleotide
ccctttattacaactgccaa

SEQ ID NO: 731 - DNA, 20 bp, Artificial Sequence
  Synthetic polynucleotide
cctttattacaactgccaaa

SEQ ID NO: 732 - DNA, 20 bp, Artificial Sequence
  Synthetic polynucleotide
ccccagatcctgtctccctt

SEQ ID NO: 733 - DNA, 20 bp, Artificial Sequence
  Synthetic polynucleotide
tacaactgccaaagggagac

SEQ ID NO: 734 - DNA, 20 bp, Artificial Sequence
  Synthetic polynucleotide
tgccaaagggagacaggatc

SEQ ID NO: 735 - DNA, 20 bp, Artificial Sequence
  Synthetic polynucleotide
gccaaagggagacaggatct

SEQ ID NO: 736 - DNA, 20 bp, Artificial Sequence
  Synthetic polynucleotide
ccaaagggagacaggatctg

SEQ ID NO: 737 - DNA, 20 bp, Artificial Sequence
  Synthetic polynucleotide
caaagggagacaggatctgg

SEQ ID NO: 738 - DNA, 20 bp, Artificial Sequence
  Synthetic polynucleotide
gggagacaggatctggggga

SEQ ID NO: 739 - DNA, 20 bp, Artificial Sequence
  Synthetic polynucleotide
agacaggatctgggggaagg

SEQ ID NO: 740 - DNA, 20 bp, Artificial Sequence
  Synthetic polynucleotide
atatatatatgtctatgtgg

SEQ ID NO: 741 - DNA, 20 bp, Artificial Sequence
  Synthetic polynucleotide
tatatatatatatgtctatg

SEQ ID NO: 742 - DNA, 20 bp, Artificial Sequence
  Synthetic polynucleotide
gattcttaatgatgatatca

SEQ ID NO: 743 - DNA, 20 bp, Artificial Sequence
  Synthetic polynucleotide
aaagcttattatattaataa

SEQ ID NO: 744 - DNA, 20 bp, Artificial Sequence
  Synthetic polynucleotide
tatatatatatagaataaga

SEQ ID NO: 745 - DNA, 20 bp, Artificial Sequence
  Synthetic polynucleotide
agaataagatggaagaaaac

SEQ ID NO: 746 - DNA, 20 bp, Artificial Sequence
  Synthetic polynucleotide
aacttggctgtggtggaagg

SEQ ID NO: 747 - DNA, 20 bp, Artificial Sequence
  Synthetic polynucleotide
aggaacttggctgtggtgga

SEQ ID NO: 748 - DNA, 20 bp, Artificial Sequence
  Synthetic polynucleotide
caaaaggaacttggctgtgg

SEQ ID NO: 749 - DNA, 20 bp, Artificial Sequence
  Synthetic polynucleotide
tgccaaaaggaacttggctg

SEQ ID NO: 750 - DNA, 20 bp, Artificial Sequence
  Synthetic polynucleotide
ttcaactgccaaaaggaact

SEQ ID NO: 751 - DNA, 20 bp, Artificial Sequence
  Synthetic polynucleotide
caccacagccaagttccttt

SEQ ID NO: 752 - DNA, 20 bp, Artificial Sequence
  Synthetic polynucleotide
cgtttacttcaactgccaaa

SEQ ID NO: 753 - DNA, 20 bp, Artificial Sequence
  Synthetic polynucleotide
ttgaagtaaacgacagcaac

SEQ ID NO: 754 - DNA, 20 bp, Artificial Sequence
  Synthetic polynucleotide
cgacagcaacaggcaatcca

SEQ ID NO: 755 - DNA, 20 bp, Artificial Sequence
  Synthetic polynucleotide
tttgctgagatctacaacct

SEQ ID NO: 756 - DNA, 20 bp, Artificial Sequence
  Synthetic polynucleotide
ttgaagttatgtcgccaccc

SEQ ID NO: 757 - DNA, 20 bp, Artificial Sequence
  Synthetic polynucleotide
agacagtgtatgcacctggg

SEQ ID NO: 758 - DNA, 20 bp, Artificial Sequence
  Synthetic polynucleotide
ggcagacagtgtatgcacct

SEQ ID NO: 759 - DNA, 20 bp, Artificial Sequence
  Synthetic polynucleotide
aggcagacagtgtatgcacc

SEQ ID NO: 760 - DNA, 20 bp, Artificial Sequence
  Synthetic polynucleotide
ttctgtttcgacagttagga

SEQ ID NO: 761 - DNA, 20 bp, Artificial Sequence
  Synthetic polynucleotide
tttgttctgtttcgacagtt

SEQ ID NO: 762 - DNA, 20 bp, Artificial Sequence
  Synthetic polynucleotide
tagcgatgtattcccaccgt

SEQ ID NO: 763 - DNA, 20 bp, Artificial Sequence
  Synthetic polynucleotide
gagagccctagaccgacggt

SEQ ID NO: 764 - DNA, 20 bp, Artificial Sequence
  Synthetic polynucleotide
cgagagccctagaccgacgg

SEQ ID NO: 765 - DNA, 20 bp, Artificial Sequence
  Synthetic polynucleotide
ctacgagagccctagaccga

SEQ ID NO: 766 - DNA, 20 bp, Artificial Sequence
  Synthetic polynucleotide
tgtattcccaccgtcggtct

SEQ ID NO: 767 - DNA, 20 bp, Artificial Sequence
  Synthetic polynucleotide
gtattcccaccgtcggtcta

SEQ ID NO: 768 - DNA, 20 bp, Artificial Sequence
  Synthetic polynucleotide
gtgtatatacgcatatatgt

SEQ ID NO: 769 - DNA, 20 bp, Artificial Sequence
  Synthetic polynucleotide
ttaacttatcgttaacttat

SEQ ID NO: 770 - DNA, 20 bp, Artificial Sequence
  Synthetic polynucleotide
agtgtattaatatgtaccaa

SEQ ID NO: 771 - DNA, 20 bp, Artificial Sequence
  Synthetic polynucleotide
gcaactacgggaacagcctt

SEQ ID NO: 772 - DNA, 20 bp, Artificial Sequence
  Synthetic polynucleotide
actgatattcccgcaactac

SEQ ID NO: 773 - DNA, 20 bp, Artificial Sequence
  Synthetic polynucleotide
aaaggctgttcccgtagttg

SEQ ID NO: 774 - DNA, 20 bp, Artificial Sequence
  Synthetic polynucleotide
gactgatattcccgcaacta

SEQ ID NO: 775 - DNA, 20 bp, Artificial Sequence
  Synthetic polynucleotide
aaggctgttcccgtagttgc

SEQ ID NO: 776 - DNA, 20 bp, Artificial Sequence
  Synthetic polynucleotide
agtgtataactttttcaggt

SEQ ID NO: 777 - DNA, 20 bp, Artificial Sequence
  Synthetic polynucleotide
tttaagtgtataactttttc

SEQ ID NO: 778 - DNA, 20 bp, Artificial Sequence
  Synthetic polynucleotide
ataaaattaataattattag

SEQ ID NO: 779 - DNA, 20 bp, Artificial Sequence
  Synthetic polynucleotide
ttagtggcacacactattga

SEQ ID NO: 780 - DNA, 20 bp, Artificial Sequence
  Synthetic polynucleotide
aatattactaacgaatgaca

SEQ ID NO: 781 - DNA, 20 bp, Artificial Sequence
  Synthetic polynucleotide
tgtacttagtaatatcattt

SEQ ID NO: 782 - DNA, 20 bp, Artificial Sequence
  Synthetic polynucleotide
atcttaaagaatatttgcat

SEQ ID NO: 783 - DNA, 20 bp, Artificial Sequence
  Synthetic polynucleotide
tgcaaatattctttaagatt

SEQ ID NO: 784 - DNA, 20 bp, Artificial Sequence
  Synthetic polynucleotide
tctttaagattaggttcgct

SEQ ID NO: 785 - DNA, 20 bp, Artificial Sequence
  Synthetic polynucleotide
ctttaagattaggttcgcta

SEQ ID NO: 786 - DNA, 20 bp, Artificial Sequence
  Synthetic polynucleotide
agattaggttcgctagggtt

SEQ ID NO: 787 - DNA, 20 bp, Artificial Sequence
  Synthetic polynucleotide
actgttaatgttgcaggtta

SEQ ID NO: 788 - DNA, 20 bp, Artificial Sequence
  Synthetic polynucleotide
attaatactgttaatgttgc

SEQ ID NO: 789 - DNA, 20 bp, Artificial Sequence
  Synthetic polynucleotide
cgtatacttaattttatgct

SEQ ID NO: 790 - DNA, 20 bp, Artificial Sequence
  Synthetic polynucleotide
tatttgtgcaattataagtt

SEQ ID NO: 791 - DNA, 20 bp, Artificial Sequence
  Synthetic polynucleotide
gtcaaagacccacaacaata

SEQ ID NO: 792 - DNA, 20 bp, Artificial Sequence
  Synthetic polynucleotide
taagttaggccttattgttg

SEQ ID NO: 793 - DNA, 20 bp, Artificial Sequence
  Synthetic polynucleotide
aagttaggccttattgttgt

SEQ ID NO: 794 - DNA, 20 bp, Artificial Sequence
  Synthetic polynucleotide
ttaacagtgttaattttaaa

SEQ ID NO: 795 - DNA, 20 bp, Artificial Sequence
  Synthetic polynucleotide
ttggtttatgaaaaattagt

SEQ ID NO: 796 - DNA, 20 bp, Artificial Sequence
  Synthetic polynucleotide
cttggtttatgaaaaattag

SEQ ID NO: 797 - DNA, 20 bp, Artificial Sequence
  Synthetic polynucleotide
tgcctatataactaaattct

SEQ ID NO: 798 - DNA, 20 bp, Artificial Sequence
  Synthetic polynucleotide
aaccaagaatttagttatat

SEQ ID NO: 799 - DNA, 20 bp, Artificial Sequence
  Synthetic polynucleotide
tctcaggactttctacactt

SEQ ID NO: 800 - DNA, 20 bp, Artificial Sequence
  Synthetic polynucleotide
agattggtggagatgatctc

SEQ ID NO: 801 - DNA, 20 bp, Artificial Sequence
  Synthetic polynucleotide
tcacctagggttgagattgg

SEQ ID NO: 802 - DNA, 20 bp, Artificial Sequence
  Synthetic polynucleotide
aactcacctagggttgagat

SEQ ID NO: 803 - DNA, 20 bp, Artificial Sequence
  Synthetic polynucleotide
tctccaccaatctcaaccct

SEQ ID NO: 804 - DNA, 20 bp, Artificial Sequence
  Synthetic polynucleotide
tcaagttgctaactcaccta

SEQ ID NO: 805 - DNA, 20 bp, Artificial Sequence
  Synthetic polynucleotide
ctcaagttgctaactcacct

SEQ ID NO: 806 - DNA, 20 bp, Artificial Sequence
  Synthetic polynucleotide
ggtgagttagcaacttgaga

SEQ ID NO: 807 - DNA, 20 bp, Artificial Sequence
  Synthetic polynucleotide
tagcaacttgagaaggtcta

SEQ ID NO: 808 - DNA, 20 bp, Artificial Sequence
  Synthetic polynucleotide
gggattagggctattaacaa

SEQ ID NO: 809 - DNA, 20 bp, Artificial Sequence
  Synthetic polynucleotide
agtatcatatagtgggatta

SEQ ID NO: 810 - DNA, 20 bp, Artificial Sequence
  Synthetic polynucleotide
aagtatcatatagtgggatt

SEQ ID NO: 811 - DNA, 20 bp, Artificial Sequence
  Synthetic polynucleotide
ctcttaaagtatcatatagt

SEQ ID NO: 812 - DNA, 20 bp, Artificial Sequence
  Synthetic polynucleotide
tctcttaaagtatcatatag

SEQ ID NO: 813 - DNA, 20 bp, Artificial Sequence
  Synthetic polynucleotide
agagagacagtttttgtgaa

SEQ ID NO: 814 - DNA, 20 bp, Artificial Sequence
  Synthetic polynucleotide
gagagacagtttttgtgaaa

SEQ ID NO: 815 - DNA, 20 bp, Artificial Sequence
  Synthetic polynucleotide
gagagtgataagtgatgtgt

SEQ ID NO: 816 - DNA, 20 bp, Artificial Sequence
  Synthetic polynucleotide
atagggatcctcttgtggtt

SEQ ID NO: 817 - DNA, 20 bp, Artificial Sequence
  Synthetic polynucleotide
aatagggatcctcttgtggt

SEQ ID NO: 818 - DNA, 20 bp, Artificial Sequence
  Synthetic polynucleotide
atcactctcccaaccacaag

SEQ ID NO: 819 - DNA, 20 bp, Artificial Sequence
  Synthetic polynucleotide
ggctaatagggatcctcttg

SEQ ID NO: 820 - DNA, 20 bp, Artificial Sequence
  Synthetic polynucleotide
atatatataaatggctaata

SEQ ID NO: 821 - DNA, 20 bp, Artificial Sequence
  Synthetic polynucleotide
tatatatataaatggctaat

SEQ ID NO: 822 - DNA, 20 bp, Artificial Sequence
  Synthetic polynucleotide
atagttgttatatatataaa

SEQ ID NO: 823 - DNA, 20 bp, Artificial Sequence
  Synthetic polynucleotide
agagagatagagagaagaag

SEQ ID NO: 824 - DNA, 20 bp, Artificial Sequence
  Synthetic polynucleotide
gagagatagagagaagaaga

SEQ ID NO: 825 - DNA, 20 bp, Artificial Sequence
  Synthetic polynucleotide
aagagggtttgatgagtttt

SEQ ID NO: 826 - DNA, 20 bp, Artificial Sequence
  Synthetic polynucleotide
gagtttttggttgtataatt

SEQ ID NO: 827 - DNA, 20 bp, Artificial Sequence
  Synthetic polynucleotide
tttttggttgtataatttgg

SEQ ID NO: 828 - DNA, 20 bp, Artificial Sequence
  Synthetic polynucleotide
ggctgacattcaacaattta

SEQ ID NO: 829 - DNA, 20 bp, Artificial Sequence
  Synthetic polynucleotide
taaagttattgggagttgtg

SEQ ID NO: 830 - DNA, 20 bp, Artificial Sequence
  Synthetic polynucleotide
ccgactactgtaaagttatt

SEQ ID NO: 831 - DNA, 20 bp, Artificial Sequence
  Synthetic polynucleotide
gccgactactgtaaagttat

SEQ ID NO: 832 - DNA, 20 bp, Artificial Sequence
  Synthetic polynucleotide
cccaataactttacagtagt

SEQ ID NO: 833 - DNA, 20 bp, Artificial Sequence
  Synthetic polynucleotide
ttatataattcttatagcaa

SEQ ID NO: 834 - DNA, 20 bp, Artificial Sequence
  Synthetic polynucleotide
aaaggttctaatatatattg

SEQ ID NO: 835 - DNA, 20 bp, Artificial Sequence
  Synthetic polynucleotide
ttagcttttgtaacatcaaa

SEQ ID NO: 836 - DNA, 20 bp, Artificial Sequence
  Synthetic polynucleotide
ggctgacattcaacaattta

SEQ ID NO: 837 - DNA, 20 bp, Artificial Sequence
  Synthetic polynucleotide
gttttggttatataatttgg

SEQ ID NO: 838 - DNA, 20 bp, Artificial Sequence
  Synthetic polynucleotide
tgagttttggttatataatt

SEQ ID NO: 839 - DNA, 20 bp, Artificial Sequence
  Synthetic polynucleotide
gaagagggtttgatgagttt

SEQ ID NO: 840 - DNA, 20 bp, Artificial Sequence
  Synthetic polynucleotide
tgtagagagagatcagaaga

SEQ ID NO: 841 - DNA, 20 bp, Artificial Sequence
  Synthetic polynucleotide
atgtagagagagatcagaag

SEQ ID NO: 842 - DNA, 20 bp, Artificial Sequence
  Synthetic polynucleotide
atagttgttatatatataaa

SEQ ID NO: 843 - DNA, 20 bp, Artificial Sequence
  Synthetic polynucleotide
tatatatataaatggctaat

SEQ ID NO: 844 - DNA, 20 bp, Artificial Sequence
  Synthetic polynucleotide
atatatataaatggctaata

SEQ ID NO: 845 - DNA, 20 bp, Artificial Sequence
  Synthetic polynucleotide
ggctaatagggatcctcttg

SEQ ID NO: 846 - DNA, 20 bp, Artificial Sequence
  Synthetic polynucleotide
atcactctcccaaccacaag

SEQ ID NO: 847 - DNA, 20 bp, Artificial Sequence
  Synthetic polynucleotide
aatagggatcctcttgtggt

SEQ ID NO: 848 - DNA, 20 bp, Artificial Sequence
  Synthetic polynucleotide
atagggatcctcttgtggtt

SEQ ID NO: 849 - DNA, 20 bp, Artificial Sequence
  Synthetic polynucleotide
gagagtgataagtgatgtgt

SEQ ID NO: 850 - DNA, 20 bp, Artificial Sequence
  Synthetic polynucleotide
gagacacagtttttgtgaaa

SEQ ID NO: 851 - DNA, 20 bp, Artificial Sequence
  Synthetic polynucleotide
agagacacagtttttgtgaa

SEQ ID NO: 852 - DNA, 20 bp, Artificial Sequence
  Synthetic polynucleotide
tctcttaaagtatcatatag

SEQ ID NO: 853 - DNA, 20 bp, Artificial Sequence
  Synthetic polynucleotide
ctcttaaagtatcatatagt

SEQ ID NO: 854 - DNA, 20 bp, Artificial Sequence
  Synthetic polynucleotide
aagtatcatatagtgggaat

SEQ ID NO: 855 - DNA, 20 bp, Artificial Sequence
  Synthetic polynucleotide
agtatcatatagtgggaata

SEQ ID NO: 856 - DNA, 20 bp, Artificial Sequence
  Synthetic polynucleotide
gggaatagggctattaacaa

SEQ ID NO: 857 - DNA, 20 bp, Artificial Sequence
  Synthetic polynucleotide
tagcaacttgagaaggtcta

SEQ ID NO: 858 - DNA, 20 bp, Artificial Sequence
  Synthetic polynucleotide
ggtgagttagcaacttgaga

SEQ ID NO: 859 - DNA, 20 bp, Artificial Sequence
  Synthetic polynucleotide
ctcaagttgctaactcacct

SEQ ID NO: 860 - DNA, 20 bp, Artificial Sequence
  Synthetic polynucleotide
tcaagttgctaactcaccta

SEQ ID NO: 861 - DNA, 20 bp, Artificial Sequence
  Synthetic polynucleotide
tctccaccaatctcaaccct

SEQ ID NO: 862 - DNA, 20 bp, Artificial Sequence
  Synthetic polynucleotide
aactcacctagggttgagat

SEQ ID NO: 863 - DNA, 20 bp, Artificial Sequence
  Synthetic polynucleotide
tcacctagggttgagattgg

SEQ ID NO: 864 - DNA, 20 bp, Artificial Sequence
  Synthetic polynucleotide
agattggtggagatgatctc

SEQ ID NO: 865 - DNA, 20 bp, Artificial Sequence
  Synthetic polynucleotide
tctcaggactttctacactt

SEQ ID NO: 866 - DNA, 20 bp, Artificial Sequence
  Synthetic polynucleotide
attaatactgttaatgttgc

SEQ ID NO: 867 - DNA, 20 bp, Artificial Sequence
  Synthetic polynucleotide
actgttaatgttgcaggtta

SEQ ID NO: 868 - DNA, 20 bp, Artificial Sequence
  Synthetic polynucleotide
tcgctagggttaggagcatc

SEQ ID NO: 869 - DNA, 20 bp, Artificial Sequence
  Synthetic polynucleotide
agattaggttcgctagggtt

SEQ ID NO: 870 - DNA, 20 bp, Artificial Sequence
  Synthetic polynucleotide
ctttaagattaggttcgcta

SEQ ID NO: 871 - DNA, 20 bp, Artificial Sequence
  Synthetic polynucleotide
tctttaagattaggttcgct

SEQ ID NO: 872 - DNA, 20 bp, Artificial Sequence
  Synthetic polynucleotide
tgcaaatattctttaagatt

SEQ ID NO: 873 - DNA, 20 bp, Artificial Sequence
  Synthetic polynucleotide
atcttaaagaatatttgcat

SEQ ID NO: 874 - DNA, 20 bp, Artificial Sequence
  Synthetic polynucleotide
aacttttagatatattactt

SEQ ID NO: 875 - DNA, 20 bp, Artificial Sequence
  Synthetic polynucleotide
tatattacttaggaatcaca

SEQ ID NO: 876 - DNA, 20 bp, Artificial Sequence
  Synthetic polynucleotide
tagtggcacacacttattga

SEQ ID NO: 877 - DNA, 20 bp, Artificial Sequence
  Synthetic polynucleotide
taaaaattaataattattag

SEQ ID NO: 878 - DNA, 20 bp, Artificial Sequence
  Synthetic polynucleotide
ttaaagtgtataattttgtc

SEQ ID NO: 879 - DNA, 20 bp, Artificial Sequence
  Synthetic polynucleotide
agtgtataattttgtcaggt

SEQ ID NO: 880 - DNA, 20 bp, Artificial Sequence
  Synthetic polynucleotide
aagcctgttcccgtagttgc

SEQ ID NO: 881 - DNA, 20 bp, Artificial Sequence
  Synthetic polynucleotide
gactgatattcccgcaacta

SEQ ID NO: 882 - DNA, 20 bp, Artificial Sequence
  Synthetic polynucleotide
aaagcctgttcccgtagttg

SEQ ID NO: 883 - DNA, 20 bp, Artificial Sequence
  Synthetic polynucleotide
actgatattcccgcaactac

SEQ ID NO: 884 - DNA, 20 bp, Artificial Sequence
  Synthetic polynucleotide
attcccgcaactacgggaac

SEQ ID NO: 885 - DNA, 20 bp, Artificial Sequence
  Synthetic polynucleotide
gcaactacgggaacaggctt

SEQ ID NO: 886 - DNA, 20 bp, Artificial Sequence
  Synthetic polynucleotide
ttaacttaccattaacttat

SEQ ID NO: 887 - DNA, 20 bp, Artificial Sequence
  Synthetic polynucleotide
aaataacaccaataagttaa

SEQ ID NO: 888 - DNA, 20 bp, Artificial Sequence
  Synthetic polynucleotide
tgtgtgtgatgatttaatga

SEQ ID NO: 889 - DNA, 20 bp, Artificial Sequence
  Synthetic polynucleotide
gtgtgtgatgatttaatgat

SEQ ID NO: 890 - DNA, 20 bp, Artificial Sequence
  Synthetic polynucleotide
atgggcgtacgcatatatgt

SEQ ID NO: 891 - DNA, 20 bp, Artificial Sequence
  Synthetic polynucleotide
gaattcccaccgtcggtctt

SEQ ID NO: 892 - DNA, 20 bp, Artificial Sequence
  Synthetic polynucleotide
tgaattcccaccgtcggtct

SEQ ID NO: 893 - DNA, 20 bp, Artificial Sequence
  Synthetic polynucleotide
ctacgagagcccaagaccga

SEQ ID NO: 894 - DNA, 20 bp, Artificial Sequence
  Synthetic polynucleotide
cgagagcccaagaccgacgg

SEQ ID NO: 895 - DNA, 20 bp, Artificial Sequence
  Synthetic polynucleotide
gagagcccaagaccgacggt

SEQ ID NO: 896 - DNA, 20 bp, Artificial Sequence
  Synthetic polynucleotide
aagcgatgaattcccaccgt

SEQ ID NO: 897 - DNA, 20 bp, Artificial Sequence
  Synthetic polynucleotide
tttgttctgtttcgacagtt

SEQ ID NO: 898 - DNA, 20 bp, Artificial Sequence
  Synthetic polynucleotide
ttctgtttcgacagttagga

SEQ ID NO: 899 - DNA, 20 bp, Artificial Sequence
  Synthetic polynucleotide
aggcagacagtgtatgcacc

SEQ ID NO: 900 - DNA, 20 bp, Artificial Sequence
  Synthetic polynucleotide
ggcagacagtgtatgcaccc

SEQ ID NO: 901 - DNA, 20 bp, Artificial Sequence
  Synthetic polynucleotide
agacagtgtatgcacccggg

SEQ ID NO: 902 - DNA, 20 bp, Artificial Sequence
  Synthetic polynucleotide
ttgaagttatgtcgccaccc

SEQ ID NO: 903 - DNA, 20 bp, Artificial Sequence
  Synthetic polynucleotide
gttgaagttatgtcgccacc

SEQ ID NO: 904 - DNA, 20 bp, Artificial Sequence
  Synthetic polynucleotide
tttgctgaaatctacaacct

SEQ ID NO: 905 - DNA, 20 bp, Artificial Sequence
  Synthetic polynucleotide
cggcagcaacaggcaatcca

SEQ ID NO: 906 - DNA, 20 bp, Artificial Sequence
  Synthetic polynucleotide
ttgaagtaaacggcagcaac

SEQ ID NO: 907 - DNA, 20 bp, Artificial Sequence
  Synthetic polynucleotide
cttttggcagttgaagtaaa

SEQ ID NO: 908 - DNA, 20 bp, Artificial Sequence
  Synthetic polynucleotide
cgtttacttcaactgccaaa

SEQ ID NO: 909 - DNA, 20 bp, Artificial Sequence
  Synthetic polynucleotide
caccacagccaagttccttt

SEQ ID NO: 910 - DNA, 20 bp, Artificial Sequence
  Synthetic polynucleotide
ttcaactgccaaaaggaact

SEQ ID NO: 911 - DNA, 20 bp, Artificial Sequence
  Synthetic polynucleotide
tgccaaaaggaacttggctg

SEQ ID NO: 912 - DNA, 20 bp, Artificial Sequence
  Synthetic polynucleotide
caaaaggaacttggctgtgg

SEQ ID NO: 913 - DNA, 20 bp, Artificial Sequence
  Synthetic polynucleotide
aggaacttggctgtggtgga

SEQ ID NO: 914 - DNA, 20 bp, Artificial Sequence
  Synthetic polynucleotide
aacttggctgtggtggaagg

SEQ ID NO: 915 - DNA, 20 bp, Artificial Sequence
  Synthetic polynucleotide
gacatatatagatagataga

SEQ ID NO: 916 - DNA, 20 bp, Artificial Sequence
  Synthetic polynucleotide
tgtatcatcaataatatata

SEQ ID NO: 917 - DNA, 64 bp, Cannabis sativa
tgtcagataatcctcttattattggaagagtgataggagatgtggttgatgttttctctccaac

SEQ ID NO: 918 - DNA, 63 bp, Cannabis sativa
tgtcagataatcctcttattattggaagagtgataggaatgtggttgatgttttctctccaac

SEQ ID NO: 919 - DNA, 62 bp, Cannabis sativa
tgtcagataatcctcttattattggaagagtgataggatgtggttgatgttttctctccaac

SEQ ID NO: 920 - DNA, 65 bp, Cannabis sativa
tgtcagataatcctcttattattggaagagtgataggagaatgtggttgatgttttctctccaac

SEQ ID NO: 921 - DNA, 59 bp, Cannabis sativa
tgtcagataatcctcttattattggaagagtgatatgtggttgatgttttctctccaac

SEQ ID NO: 922 - DNA, 60 bp, Cannabis sativa
tgtcagataatcctcttattattggaagagtgataatgtggttgatgttttctctccaac

SEQ ID NO: 923 - DNA, 64 bp, Cannabis sativa
ttgatgttttctctccaaccctaaaatttacagtgacttacaactcaaacaagaaagtctacaa

SEQ ID NO: 924 - DNA, 63 bp, Cannabis sativa
ttgatgttttctctccaaccctaaatttacagtgacttacaactcaaacaagagagtctacaa

SEQ ID NO: 925 - DNA, 18 bp, Cannabis sativa
ttgatgttttctctacaa

SEQ ID NO: 926 - DNA, 64 bp, Cannabis sativa
tccttccacaattactattaagccaaaagttgaggttcatggtggtgacatgagatcctttttc

SEQ ID NO: 927 - DNA, 65 bp, Cannabis sativa
tccttccacaattactattaagccaaaagttgaggttcaatggtggtgacatgagatcctttttc

SEQ ID NO: 928 - DNA, 63 bp, Cannabis sativa
tccttccacaattactatcaagccaaaagttgaggttcaggtggtgacatgagatcctttttc

SEQ ID NO: 929 - DNA, 58 bp, Cannabis sativa
tccttccacaattactattaagccaaaagttgatggtggtgacatgagatcctttttc

SEQ ID NO: 930 - DNA, 62 bp, Cannabis sativa
tccttccacaattactattaagccaaaagttgaggttcggtggtgacatgagatcctttttc

SEQ ID NO: 931 - DNA, 61 bp, Cannabis sativa
tccttccacaattactattaagccaaaagttgaggttggtggtgacatgagatcctttttc

SEQ ID NO: 932 - DNA, 62 bp, Cannabis sativa
tccttccacaattactattaagccaaaagttgaggtttggtggtgacatgagatcctttttc

SEQ ID NO: 933 - DNA, 63 bp, Cannabis sativa
tccttccacaattactattaagccaaaagttgaggttctggtggtgacatgagatcctttttc

SEQ ID NO: 934 - DNA, 174 bp, Cannabis sativa
aatcctcttattattggaagagtgataggagatgtggttgatgttttctctccaaccctaaaatttacagtgacttacaactcaaacaagagagtctacaatggccatgagttgtttccttccacaattactattaagccaaaagttgaggttcatggtggtgacatgagatcc

SEQ ID NO: 935 - DNA, 50 bp, Cannabis sativa
aatcctcttattattggaagagtgataggagtggtggtgacatgagatcc

SEQ ID NO: 936 - DNA, 51 bp, Cannabis sativa
aatcctcttattattggaagagtgataggagatggtggtgacatgagatcc

SEQ ID NO: 937 - DNA, 143 bp, Cannabis sativa
aatcctcttattattggaagagtgataggagatttacagtgacttacaactcaaacaagagagtctacaatggccatgagttgtttccttccacaattactattaagccaaaagttgaggttcatggtggtgacatgagatcc

SEQ ID NO: 938 - DNA, 83 bp, Cannabis sativa
aatcctcttattattggaagagtgataggagaatgtggttgatgttttctctccaaccctaaaatggtggtgacatgagatcc

(19) United States
(12) Patent Application Publication
(10) Pub. No.: US 2022/0186243 A1
(43) Pub. Date: Jun. 16, 2022

(54) CANNABIS PLANTS WITH IMPROVED YIELD

(71) Applicant: BetterSeeds Ltd., Givat Chen (IL)

(72) Inventors: Tal SHERMAN, Rehovot (IL); Ido MARGALIT, Gan Yavne (IL); Shira COREM, Sdei Hemed (IL)

(73) Assignee: BetterSeeds Ltd., Givat Chen (IL)

(21) Appl. No.: 17/455,846

(22) Filed: Nov. 19, 2021

Related U.S. Application Data
(63) Continuation-in-part of application No. PCT/IL2020/050592, filed on May 27, 2020.
(60) Provisional application No. 62/854,036, filed on May 29, 2019.
(60) Provisional application No. 63/030,500, filed on May 27, 2020.

(51) Int. Cl. C12N 15/82; C12Q 1/6895

(52) U.S. Cl. CPC C12N 15/8261; C12N 15/8213; C12Q 2600/156; C12Q 2600/13; C12Q 1/6895

(57) ABSTRACT

The present invention discloses a modified Cannabis plant exhibiting at least one improved domestication trait compared with wild type Cannabis, wherein the modified plant comprises at least one mutated Cannabis SELF PRUNING (SP) (CsSP) gene and/or at least one mutated Cannabis SELF PRUNING 5G (SP5G) (CsSP5G) gene. The present invention further discloses methods for production of the aforementioned modified Cannabis plant using genome modification.

### SEQUENCE LISTING

The Sequence Listing submitted in text format (.txt) filed on Mar. 4, 2022, named “SequenceListing.txt”, created on Feb. 23, 2022 (225 KB), is incorporated herein by reference.

### FIELD OF THE INVENTION

The present disclosure relates to conferring desirable agronomic traits in Cannabis plants. More particularly, the current invention pertains to producing Cannabis plants with improved yield traits by manipulating genes controlling day-length sensitivity and plant architecture.

### BACKGROUND OF THE INVENTION

Cannabis is one of the oldest domesticated plants with evidence of being used by a vast array of ancient cultures. It is thought to have originated from central Asia from which it was spread by humans to China, Europe, the Middle East and the Americas. Thus, Cannabis has been bred by many different cultures for various uses such as food, fiber and medicine since the dawn of agricultural societies. In the last few decades, Cannabis breeding has stopped as it became illegal and non-economic to do so. With the recent legislation converting Cannabis back to legality, there is a growing need for the implementation of new and advanced breeding techniques in future Cannabis breeding programs. This will allow speeding up the long process of classical breeding and accelerate reaching new and genetically improved Cannabis varieties for fiber, food and medicine products. Developing and implementing molecular biology tools to support the breeders, will allow creating new traits and tracking the movement of such desired traits across breeders germplasm.

Currently, breeding of Cannabis plants is mostly done by small Cannabis growers. There is very limited if any molecular tools supporting or leading the breeding process. Traditional Cannabis breeding is done by mixing breeding material with hope to find the desired traits and phenotypes by random crosses. These methods have allowed the construction of the leading Cannabis varieties on the market today. As the cultivation of Cannabis intensifies in protected structures such as greenhouses and closed growth chambers, such an environment encourages the prevalence of certain diseases, with the lead cause being fungi.

One of the most important determinants of crop productivity is plant architecture. For many crops, artificial selection for modified shoot architectures provided critical steps towards improving yield, followed by innovations enabling large- scale field production. A prominent example is tomato, in which the discovery of a mutation in the antiflorigen-encoding self-pruning gene (sp), led to determinate plants that provided a burst of flowering and synchronized fruit ripening, permitting mechanical harvesting.

In addition, day-length sensitivity in crops limits their geographical range of cultivation, and thus modification of the photoperiod response was critical for their domestication.

PCT application WO2017180474 discloses a tomato plant that is a sp5g sp double mutant and that flowers earlier than the corresponding sp [sibling] tomato plant, as measured with reference to the number of leaves produced prior to appearance of first inflorescence.

The publication of Soyk et al (2016), Nature Genetics, “Variation in the flowering gene SELF PRUNING 5G promotes day-neutrality and early yield in tomato” shows that loss of day-length-sensitive flowering in tomato was driven by the florigen paralog and flowering repressor SELF-PRUNING 5G (SP5G). This publication reports that CRISPR/Cas9-engineered mutations in SP5G cause rapid flowering and enhance the compact determinate growth habit of field tomatoes, resulting in a quick burst of flower production that translates to an early yield. However, these engineered tomato plants showed total yield reduction as compared to sp mutated tomato plants.

The publication of Li et al (2018), nature biotechnology, “Domestication of wild tomato is accelerated by genome editing” teach the assembly of a set of six gRNAs to edit four genes (SlCLV3, SlWUS, SP and SP5G) into one construct. The construct was transformed into four S. pimpinellifolium accessions, all of which are resistant to bacterial spot disease, and two of which are salt tolerant. Small indels and large insertions have been identified in the targeted regulatory regions of SlCLV3 and SlWUS in TO and their Ti mutant plants. It was reported in this publication that although SP and SP5G are crucial for improving the harvest index, the limited allelic variation has hampered efforts to optimize this trait. It was further reported that locule number was not increased in TO and Ti plants with large insertions and inversions in the targeted SlCLV3 promoter region. One explanation for this finding is that the targeted region of the SlCLV3 promoter may not be essential for regulating SlCLV3 transcription. Alternatively, it was suggested that disruption of regions (gRNA-5) flanking the CArG element downstream of SlWUS may have decreased its transcription and counteracted the effects of mutation of SlCLV3, owing to a negative feedback loop of CLV3-WUS in controlling stem cell proliferation.

The publication of Zsogon et al (2018), nature biotechnology, “De novo domestication of wild tomato using genome editing” discloses a devised CRISPR—Cas9 genome engineering strategy to combine agronomically desirable traits with useful traits presented in Solanum pimpinellifolium wild lines. The four edited genes were SELF-PRUNING (SP), OVATE (0), FRUIT WEIGHT 2.2 (FW2.2) and LYCOPENE BETA CYCLASE (CycB).

Lemmon et al (2018), Nature Plants, “Rapid improvement of domestication traits in an orphan crop by genome editing” describes the usage of CRISPR—Cas9 to mutate orthologues of tomato domestication and improvement genes that control plant architecture, flower production and fruit size in the orphan Solanaceae crop ‘groundcherry’ (Physalis pruinosa).

It is noted, however that Cannabis architecture or earliness traits were not targeted during domestication.

In view of the above there is still a long felt and unmet need to manipulate Cannabis plant architecture and flower production in a rapid and efficient way to improve yield and reduce production costs.

### SUMMARY OF THE INVENTION

It is therefore one object of the present invention to disclose a modified Cannabis plant exhibiting at least one improved domestication trait compared with wild type Cannabis, wherein said modified plant comprises at least one mutated Cannabis SELF PRUNING (SP) (CsSP) gene selected from the group consisting of CsSP-1 having a genomic nucleotide sequence as set forth in SEQ ID NO: 1 or a functional variant thereof, CsSP-2 having a genomic nucleotide sequence as set forth in SEQ ID NO: 4 or a functional variant thereof, CsSP-3 having a genomic nucleotide sequence as set forth in SEQ ID NO: 7 or a functional variant thereof and any combination thereof, and/or at least one mutated Cannabis SELF PRUNING 5G (SP5G) (CsSP5G) gene selected from the group consisting of CsSP5G-1 having a genomic nucleotide sequence as set forth in SEQ ID NO: 10 or a functional variant thereof, CsSP5G-2 having a genomic nucleotide sequence as set forth in SEQ ID NO: 13 or a functional variant thereof, CsSP5G-3 having a genomic nucleotide sequence as set forth in SEQ ID NO: 16 or a functional variant thereof, CsSP5G-4 having a genomic nucleotide sequence as set forth in SEQ ID NO: 19 or a functional variant thereof and any combination thereof.

It is a further object of the present invention to disclose the modified Cannabis plant as defined above, wherein said functional variant has at least 75% sequence identity to said CsSP or said CsSP5G nucleotide sequence.

It is a further object of the present invention to disclose the modified Cannabis plant as defined in any of the above, wherein said mutation is introduced using mutagenesis, small interfering RNA (siRNA), microRNA (miRNA), artificial miRNA (amiRNA), DNA introgression, endonucleases or any combination thereof.

It is a further object of the present invention to disclose the modified Cannabis plant as defined in any of the above, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is a further object of the present invention to disclose the modified Cannabis plant as defined in any of the above, wherein the mutated CsSP or CsSP5G gene is a CRISPR/Cas9- induced heritable mutated allele.

It is a further object of the present invention to disclose the modified Cannabis plant as defined in any of the above, wherein said plant is homozygous for said at list one CsSP or said at list one CsSP5G mutated gene, or said plant is a Cssp Cssp5g double mutant.

It is a further object of the present invention to disclose the modified Cannabis plant as defined in any of the above, wherein at least one of the following holds true: (a) said mutation in said CsSP-1 is generated in planta via introduction of a construct comprising (i) Cas DNA and gRNA sequence selected from the group consisting of SEQ ID NO: 22-SEQ ID NO: 126 and any combination thereof, or (ii) a ribonucleoprotein (RNP) complex comprising Cas protein and gRNA sequence selected from the group consisting of SEQ ID NO: 22-126 and any combination thereof; (b) said mutation in said CsSP-2 is generated in planta via introduction of a construct comprising (i) Cas DNA and gRNA sequence selected from the group consisting of SEQ ID NO: 127-SEQ ID NO: 211 and any combination thereof, or (ii) a ribonucleoprotein (RNP) complex comprising Cas protein and gRNA sequence selected from the group consisting of SEQ ID NO: 127-211 and any combination thereof (c) said mutation in said CsSP-3 is generated in planta via introduction of a construct comprising (i) Cas DNA and gRNA sequence selected from the group consisting of SEQ ID NO: 212-SEQ ID NO: 283 and any combination thereof, or (ii) a ribonucleoprotein (RNP) complex comprising Cas protein and gRNA sequence selected from the group consisting of SEQ ID NO: 212-283 and any combination thereof; (d) said mutation in said CsSP5G-1 is generated in planta via introduction of a construct comprising (i) Cas DNA and gRNA sequence selected from the group consisting of SEQ ID NO: 284-SEQ ID NO: 516 and any combination thereof, or (ii) a ribonucleoprotein (RNP) complex comprising Cas protein and gRNA sequence selected from the group consisting of SEQ ID NO: 284-516 and any combination thereof; (e) said mutation in said CsSP5G-2 is generated in planta via introduction of a construct comprising (i) Cas DNA and gRNA sequence selected from the group consisting of SEQ ID NO: 517-SEQ ID NO: 745 and any combination thereof, or (ii) a ribonucleoprotein (RNP) complex comprising Cas protein and gRNA sequence selected from the group consisting of SEQ ID NO: 517-745 and any combination thereof; (f) said mutation in said CsSP5G-3 is generated in planta via introduction of a construct comprising (i) Cas DNA and gRNA sequence selected from the group consisting of SEQ ID NO: 746-SEQ ID NO: 828 and any combination thereof, or (ii) a ribonucleoprotein (RNP) complex comprising Cas protein and gRNA sequence selected from the group consisting of SEQ ID NO: 746-828 and any combination thereof; and (g) said mutation in said CsSP5G-4 is generated in planta via introduction of a construct comprising (i) Cas DNA and gRNA sequence selected from the group consisting of SEQ ID NO: 829-SEQ ID NO: 916 and any combination thereof, or (ii) a ribonucleoprotein (RNP) complex comprising Cas protein and gRNA sequence selected from the group consisting of SEQ ID NO: 829-916 and any combination thereof.

It is a further object of the present invention to disclose the modified Cannabis plant as defined in any of the above, wherein said plant has decreased expression levels of at least one of said CsSP genes, and/or decreased expression levels of at least one of said CsSP5G genes.

It is a further object of the present invention to disclose the modified Cannabis plant as defined in any of the above, wherein said domestication trait is selected from the group consisting of reduced flowering time, earliness, synchronous flowering, earlier flowering, suppressed or reduced day-length sensitivity, determinant or semi-determinant architecture or growth habit, early termination of sympodial cycling, suppressed sympodial shoot termination, similar sympodial shoot termination as compared to a corresponding wild type cannabis plant, earlier axillary shoot flowering, compact growth habit, reduced height, reduced number of sympodial units, adaptation to mechanical harvest, higher harvest index and any combination thereof.

It is a further object of the present invention to disclose the modified Cannabis plant as defined in any of the above, wherein said Cssp Cssp5g double mutant is characterized by having a more than additive effect on a trait selected from the group consisting of compactness, earlier axillary shoot flowering, earlier termination of sympodial cycling, harvest index and any combination thereof as compared to wild type and/or sp mutant Cannabis plants.

It is a further object of the present invention to disclose the modified Cannabis plant as defined in any of the above, wherein said modified plant comprises a mutated Cannabis self pruning (sp)-1 (Cssp-1) gene allele, said mutated allele comprising a genomic modification selected from an indel at position corresponding to position 2210, 2211, 2269, 2302, 2304 2334, 2335, 2336, and any combination thereof, of Cannabis SP-1 (CsSP-1) gene having a polynucleotide sequence corresponding to a sequence having at least 80% sequence identity to the sequence as set forth in SEQ ID NO: 1, or a functional fragment or variant thereof.

It is a further object of the present invention to disclose the modified Cannabis plant as defined in any of the above, wherein said Cssp-1 allele comprises an indel at a polynucleotide sequence corresponding to a sequence having at least 80% sequence identity to the sequence as set forth in SEQ ID NO: 1 selected from 1 bp deletion at position 2335, 2 bp deletion at position 2336, 1 bp insertion at position 2335, 5 bp deletion at position 2334, 4 bp deletion at position 2334, 1 bp deletion at position 2302, 46 bp deletion at position 2269, 1 bp insertion at position 2211, 1 bp deletion at position 2210, 6 bp deletion at position 2211, 2 bp deletion at position 2210, 3 bp deletion at position 2211, 2 bp deletion at position 2211, 1 bp deletion at position 2211, 124 bp deletion at position 2211, 123 bp deletion at position 2211, 31 bp deletion at position 2304, 91 bp deletion at position 2211 and any combination thereof.

It is a further object of the present invention to disclose the modified Cannabis plant as defined in any of the above, wherein said Cssp-1 allele comprises a polynucleotide sequence having at least 80% identity to a polynucleotide sequence selected from SEQ ID NO: 918-922, SEQ ID NO: 924-925, SEQ ID NO: 927-933, SEQ ID NO: 935-938, or a complementary sequence thereof, or any combination thereof.

It is a further object of the present invention to disclose the modified Cannabis plant as defined in any of the above, wherein said mutated Cssp-1 allele confers improved domestication trait as compared to a Cannabis plant comprising a wild type CsSP-1 allele comprising a polynucleotide sequence having at least 80% identity to a polynucleotide sequence selected from SEQ ID NO: 917, SEQ ID NO: 923, SEQ ID NO: 926, SEQ ID NO: 934 or any combination thereof, and/or having a polynucleotide sequence having at least 80% identity to the polynucleotide sequence as set forth in SEQ ID NO: 1.

It is a further object of the present invention to disclose the modified Cannabis plant as defined in any of the above, wherein said modified plant is generated via introduction of a gRNA comprising a polynucleotide sequence corresponding to a sequence selected from the group consisting of SEQ ID NO: 102, SEQ ID NO: 109 and SEQ ID NO: 112, a complementary sequence thereof, and any combination thereof.

It is a further object of the present invention to disclose a plant part, plant cell, tissue culture of regenerable cells, protoplasts or callus or plant seed of a plant as defined in any of the above.

It is a further object of the present invention to disclose a method for producing a modified Cannabis plant as defined in any of the above, said method comprises steps of genetically introducing by targeted genome modification, a loss of function mutation in at least one Cannabis SELF PRUNING (SP) (CsSP) gene selected from the group consisting of CsSP-1 having a genomic nucleotide sequence as set forth in SEQ ID NO: 1 or a functional variant thereof, CsSP-2 having a genomic nucleotide sequence as set forth in SEQ ID NO: 4 or a functional variant thereof, CsSP-3 having a genomic nucleotide sequence as set forth in SEQ ID NO: 7 or a functional variant thereof and any combination thereof, and/or at least one Cannabis SELF PRUNING 5G (SPSG) (CsSP5G) gene selected from the group consisting of CsSP5G-1 having a genomic nucleotide sequence as set forth in SEQ ID NO: 10 or a functional variant thereof, CsSP5G-2 having a genomic nucleotide sequence as set forth in SEQ ID NO: 13 or a functional variant thereof, CsSP5G-3 having a genomic nucleotide sequence as set forth in SEQ ID NO: 16 or a functional variant thereof, CsSP5G-4 having a genomic nucleotide sequence as set forth in SEQ ID NO: 19 or a functional variant thereof and any combination thereof.

It is a further object of the present invention to disclose a method of improving at least one domestication trait compared with wild type Cannabis, comprising steps of producing a modified Cannabis plant as defined in any of the above, seed or plant part thereof, that is homozygous for at least one mutated CsSP5G gene selected from the group consisting of CsSP5G-1 having a genomic nucleotide sequence as set forth in SEQ ID NO: 10 or a functional variant thereof, CsSP5G-2 having a genomic nucleotide sequence as set forth in SEQ ID NO: 13 or a functional variant thereof, CsSP5G-3 having a genomic nucleotide sequence as set forth in SEQ ID NO: 16 or a functional variant thereof, CsSP5G-4 having a genomic nucleotide sequence as set forth in SEQ ID NO: 19 or a functional variant thereof and any combination thereof in a sp background and enabling growth of said Cannabis plant, seed or plant part thereof.

It is a further object of the present invention to disclose the method as defined in any of the above, wherein said method comprises steps of: (a) identifying at least one Cannabis SP (CsSP) and/or at least one Cannabis SP5G (CsSP5G) allele; (b) synthetizing at least one guide RNA (gRNA) comprising a nucleotide sequence complementary to said at least one identified CsSP and/or CsSP5G allele; (c) transforming Cannabis plant cells with a construct comprising (a) Cas nucleotide sequence operably linked to said at least one gRNA, or (b) a ribonucleoprotein (RNP) complex comprising Cas protein and said at least one gRNA; (d) screening the genome of said transformed plant cells for induced targeted loss of function mutation in at least one of said CsSP and/or CsSP5G allele; (e) regenerating Cannabis plants carrying said loss of function mutation in at least one of said CsSP and/or CsSP5G allele; and (f) screening said regenerated plants for a Cannabis plant with improved domestication trait.

It is a further object of the present invention to disclose the method as defined in any of the above, wherein at least one of the following holds true: (a) said mutation in said CsSP-1 is generated in planta via introduction of a construct comprising (i) Cas DNA and gRNA sequence selected from the group consisting of SEQ ID NO: 22-SEQ ID NO: 126 and any combination thereof, or (ii) a ribonucleoprotein (RNP) complex comprising Cas protein and gRNA sequence selected from the group consisting of SEQ ID NO: 22-126 and any combination thereof; (b) said mutation in said CsSP-2 is generated in planta via introduction of a construct comprising (i) Cas DNA and gRNA sequence selected from the group consisting of SEQ ID NO: 127-SEQ ID NO: 211 and any combination thereof, or (ii) a ribonucleoprotein (RNP) complex comprising Cas protein and gRNA sequence selected from the group consisting of SEQ ID NO: 127-211 and any combination thereof; (c) said mutation in said CsSP-3 is generated in planta via introduction of a construct comprising (i) Cas DNA and gRNA sequence selected from the group consisting of SEQ ID NO: 212-SEQ ID NO: 283 and any combination thereof, or (ii) a ribonucleoprotein (RNP) complex comprising Cas protein and gRNA sequence selected from the group consisting of SEQ ID NO: 212-283 and any combination thereof; (d) said mutation in said CsSP5G-1 is generated in planta via introduction of a construct comprising (i) Cas DNA and gRNA sequence selected from the group consisting of SEQ ID NO: 284-SEQ ID NO: 516 and any combination thereof, or (ii) a ribonucleoprotein (RNP) complex comprising Cas protein and gRNA sequence selected from the group consisting of SEQ ID NO: 284-516 and any combination thereof; (e) said mutation in said CsSP5G-2 is generated in planta via introduction of a construct comprising (i) Cas DNA and gRNA sequence selected from the group consisting of SEQ ID NO: 517-SEQ ID NO: 745 and any combination thereof, or (ii) a ribonucleoprotein (RNP) complex comprising Cas protein and gRNA sequence selected from the group consisting of SEQ ID NO: 517-745 and any combination thereof; (f) said mutation in said CsSP5G-3 is generated in planta via introduction of a construct comprising (i) Cas DNA and gRNA sequence selected from the group consisting of SEQ ID NO: 746-SEQ ID NO: 828 and any combination thereof, or (ii) a ribonucleoprotein (RNP) complex comprising Cas protein and gRNA sequence selected from the group consisting of SEQ ID NO: 746-828 and any combination thereof; and (g) said mutation in said CsSP5G-4 is generated in planta via introduction of a construct comprising (i) Cas DNA and gRNA sequence selected from the group consisting of SEQ ID NO: 829-SEQ ID NO: 916 and any combination thereof, or (ii) a ribonucleoprotein (RNP) complex comprising Cas protein and gRNA sequence selected from the group consisting of SEQ ID NO: 829-916 and any combination thereof.

It is a further object of the present invention to disclose a Cannabis plant, plant part, plant seed, tissue culture of regenerable cells, protoplasts, callus or plant cell produced by the method as defined in any of the above.

It is a further object of the present invention to disclose an isolated polynucleotide sequence having at least 75% sequence identity to a polynucleotide sequence selected from the group consisting of SEQ ID NO: 1-2, SEQ ID NO: 4-5, SEQ ID NO: 7-8, SEQ ID NO: 10-11, SEQ ID NO: 13-14, SEQ ID NO: 16-17, SEQ ID NO: 19-20, SEQ ID NO: 22-283, SEQ ID NO: 284-916, SEQ ID NO: 918-922, SEQ ID NO: 924-925, SEQ ID NO: 927-933 and SEQ ID NO: 935-938, or an isolated amino acid sequence having at least 75% sequence similarity to amino acid sequence selected from the group consisting of SEQ ID NO: 3, SEQ ID NO: 6, SEQ ID NO: 9, SEQ ID NO: 12, SEQ ID NO: 15, SEQ ID NO: 18 and SEQ ID NO: 21.

It is a further object of the present invention to disclose a method for generating, identifying and/or screening for a Cannabis plant as defined in any of the above, comprising detecting the presence of at least one polynucleotide sequence selected from the group consisting of a sequence having at least 80% identity to SEQ ID NO: 918-922, SEQ ID NO: 924-925, SEQ ID NO: 927-933, SEQ ID NO: 935-938, or a complementary sequence thereof, and a combination thereof.

It is a further object of the present invention to disclose a detection kit for identifying a Cannabis plant with improved domestication trait by determining the presence or absence of a mutant Cssp-1 allele in a Cannabis plant, comprising a polynucleotide fragment having at least 80% identity to SEQ ID NO: 918-922, SEQ ID NO: 924-925, SEQ ID NO: 927-933, SEQ ID NO: 935-938, or a complementary sequence thereof, and any combination thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Exemplary non-limited embodiments of the disclosed subject matter will be described, with reference to the following description of the embodiments, in conjunction with the figures. The figures are generally not shown to scale and any sizes are only meant to be exemplary and not necessarily limiting. Corresponding or like elements are optionally designated by the same numerals or letters.

FIG. 1A-D is photographically presenting various Cannabis tissues transformed with GUS reporter gene, where FIG. 1A shows axillary buds, FIG. 1B mature leaf, FIG. 1C calli, and Fig. D cotyledons;

FIG. 2 is photographically presenting regenerated Cannabis tissue;

FIG. 3 is photographically presenting PCR detection of Cas9 DNA in shoots of Cannabis plants transformed using biolistics; and

FIG. 4 is illustrating in vivo specific DNA cleavage by Cas9 +gRNA (RNP) complex, as an embodiment of the present invention.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The present invention provides a modified Cannabis plant exhibiting at least one improved domestication trait compared with wild type Cannabis, wherein said modified plant comprises at least one mutated Cannabis SELF PRUNING (SP) (CsSP) gene and/or at least one mutated Cannabis SELF PRUNING 5G (SP5G) (CsSP5G) gene. The present invention further provides methods for producing the aforementioned modified Cannabis plant using genome editing or other genome modification techniques.

As the Cannabis legal market is expanding worldwide, this agricultural crop will gradually move from indoor growing facilities to simple low cost greenhouses to enable mass production at reduced operational costs. One of the major challenges facing this transition is the lack of compatible genetics (strains) adapted for green house growth.

To date, there are no Cannabis varieties with improved domestication traits on the market. Classical breeding programs dedicated to the end are virtually impossible due to limited genetic variation, legal constraints on import and export of genetic material and limited academic knowledge and gene banks involved is such projects. In addition, traditional breeding is a long process with low rates of success and certainty, as it is based on trial and error.

The solution proposed by the current invention is using genome editing such as the CRISPR/Cas system in order to create cultivated Cannabis plants with improved yield and more specifically with determinate growth habit and with significantly reduced requirement for short days period needed for induction of flowering. Breeding using genome editing allows a precise and significantly shorter breeding process in order to achieve these goals with a much higher success rate. Thus genome editing, has the potential to generate improved varieties faster and at a lower cost.

It is further noted that using genome editing is considered as non GMO by the Israeli regulator and in the US, the USDA has already classified a dozen of genome edited plant as non-regulated and non GMO (https ://www.usda.gov/media/press-releases/2018/03/28/secretary-perdue-issues-usda-statement-plant-breeding-innovation).

Legal limitations and outdated breeding techniques significantly hamper the efforts of generating new and improved Cannabis varieties fit for intensive agriculture.

In addition, Cannabis growers are using Cannabis strains that were bred for indoor cultivation and are now using those for their greenhouse operations. This situation is obviously not ideal and causes many logistic issues for the growers. For example, since Cannabis plants require short days for the induction of flowering, growers install darkening curtains in the greenhouse to control day length for the plants. This artificial darkening results in increased humidity in the greenhouse thus creating optimal conditions for fungal pathogens to spread and thrive. These conditions force growers to intensively use fungicides to control pathogen populations. With strict regulatory constraints in place across the legalized states, these conditions pose a great challenge for sustainable Cannabis production and consumer health.

In order to generate a reproducible product, Cannabis growers are currently using vegetative propagation (cloning or tissue culture). However, in conventional agricultural, genetic stability of field crops and vegetables is maintained by using F 1 hybrid seeds. These hybrids are generated by crossing homozygous parental lines.

The next step for the Cannabis industry is the adoption and use of hybrid seeds for propagation, which is common practice in the conventional seed industry (from field crops to vegetables). In addition, breeding for basic agronomic traits that are completely lacking in currently available Cannabis varieties (with an emphasis on day length sensitivity and compact growth habit) will significantly increase grower's productivity. This will allow growing and supplying high quality raw material for the Cannabis industry.

Currently, breeding of Cannabis plants is mostly done by small Cannabis growers. There is very limited if any molecular tools supporting or leading the breeding process. Traditional Cannabis breeding is done by mixing breeding material with hope to find the desired traits and phenotypes by random crosses.

In general, plants' flowering is triggered by seasonal changes in day length. However, day-length sensitivity in crops limits their geographical range of cultivation, and thus modification of the photoperiod response is critical for their domestication.

The present invention provides for the first time Cannabis plants with improved domestication traits such as plant architecture and day length sensitivity. The current invention discloses the generation of non-transgenic Cannabis plants with improved yield traits, using the genome editing technology, e.g., the CRISPR/Cas9 highly precise tool. The generated mutations can be introduced into elite or locally adapted Cannabis lines rapidly, with relatively minimal effort and investment.

Genome editing is an efficient and useful tool for increasing crop productivity, and there is particular interest in advancing manipulation of domestication genes in Cannabis wild species, which often have undesirable characteristics.

Genome-editing technologies, such as the clustered regularly interspaced short palindromic repeats (CRISPR)—CRISPR-associated protein-9 nuclease (Cas9) (CRISPR—Cas9) provide opportunities to address these deficiencies, with the aims of increasing quality and yield, improve adaptation and expand geographical ranges of cultivation.

A major obstacle for CRISPR—Cas9 plant genome editing is lack of efficient tissue culture and transformation methodologies. The present invention achieves these aims and surprisingly provides transformed and regenerated Cannabis plants with modified desirable domestication genes.

Precise editing of SELFPRUNING (SP) and SELF-PRUNING 5G (SP5G) in wild Cannabis species, as disclosed by the present invention, should serve as a first step towards generating commercially cultivable lines, without causing an associated linkage drag on other useful traits. Genome engineering could thus be applied for de novo domestication of wild species to create climate-smart crops.

To that end, guide RNAs (gRNAs) were designed for each of the target genes identified in Cannabis to induce mutations in SP and SP5G through genome editing.

It was found that simultaneous mutation of SP and SP5G converted the indeterminate architecture of Cannabis into determinate growth with early termination of sympodial cycling, thus resulting in compact Cannabis plants with intensive and almost synchronous flowering. It should be emphasized that the desirable architectures and flower production traits are produced in just one generation.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention it refers to plants of the same Cannabis species or strain or variety or to sibling plant, or one or more individuals having one or both parents in common.

The term “corresponding” or “corresponding to” or “corresponding to nucleotide sequence” or “corresponding to position” as used herein, also refers in the context of the present invention to sequence homology or sequence identity. These terms relate to two or more nucleic acid or protein sequences, that are the same or have a specified percentage of amino acid residues or nucleotides that are the same, when compared and aligned for maximum correspondence, as measured using one of the available sequence comparison algorithms or by visual inspection. If two sequences, which are to be compared with each other, differ in length, sequence identity preferably relates to the percentage of the nucleotide residues of the shorter sequence, which are identical with the nucleotide residues of the longer sequence. As used herein, the percent of identity or homology between two sequences is a function of the number of identical positions shared by the sequences, taking into account the number of gaps, and the length of each gap, which needs to be introduced for optimal alignment of the two sequences. The comparison of sequences and determination of identity percent between two sequences can be accomplished using a mathematical algorithm as known in the relevant art. According to further aspects of the invention, the term “corresponding to the nucleotide sequence” or “corresponding to position”, refers to variants, homologues and fragments of the indicated nucleotide sequence, which possess or perform the same biological function or correlates with the same phenotypic characteristic of the indicated nucleotide sequence.

Another indication that two nucleic acid sequences are substantially identical or that a sequence is “corresponding to the nucleotide sequence” is that the two molecules hybridize to each other under stringent conditions. High stringency conditions, such as high hybridization temperature and low salt in hybridization buffers, permits only hybridization between nucleic acid sequences that are highly similar, whereas low stringency conditions, such as lower temperature and high salt, allows hybridization when the sequences are less similar.

In other embodiments of the invention, such substantially identical sequences refer to polynucleotide or amino acid sequences that share at least about 80% similarity or identity, preferably at least about 90% similarity or identity, alternatively, about 95%, 96%, 97%, 98% or 99% similarity or identity to the indicated polynucleotide or amino acid sequences.

According to other aspects of the invention, the term “corresponding” refers also to complementary sequences or base pairing such that when they are aligned antiparallel to each other, the nucleotide bases at each position in the sequences will be complementary. The degree of complementarity between two nucleic acid strands may vary.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell, or as a part of higher organized unit such as, for example, plant tissue, a plant organ, or a whole plant.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as a root, stem, leaf, flower, flower bud, or embryo.

The term “Plant tissue” as used herein means a group of plant cells organized into a structural and functional unit. Any tissue of a plant in planta or in culture is included. This term includes, but is not limited to, whole plants, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non limiting manner to offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds, i.e. loss of function mutation in at least one CsSP gene or at least one CsSP5G gene.

The term “Cannabis” refers hereinafter to a genus of flowering plants in the family Cannabaceae. Cannabis is an annual, dioecious, flowering herb that includes, but is not limited to three different species, Cannabis sativa, Cannabis indica and Cannabis ruderalis. The term also refers to hemp. Cannabis plants produce a group of chemicals called cannabinoids. Cannabinoids, terpenoids, and other compounds are secreted by glandular trichomes that occur most abundantly on the floral calyxes and bracts of female Cannabis plants.

The term ‘SELF-PRUNING’ or ‘SP’ in the context of the present invention refers to a gene which encodes a flowering repressor that modulates sympodial growth. It is herein shown that mutations in the SP orthologue cause an acceleration of sympodial cycling and shoot termination. It is further acknowledged that the SELF PRUNING (SP) gene controls the regularity of the vegetative-reproductive switch along the compound shoot of, for example, tomato and thus conditions the ‘determinate’ (sp/sp) and ‘indeterminate’ (SP) growth habits of the plant. SP is a developmental regulator which is homologous to CENTRORADIALIS (CEN) from Antirrhinum and TERMINAL FLOWER 1 (TFL1) and FLOWERING LOCUS T (FT) from Arabidopsis.

The present invention discloses that SP is a member of a gene family in Cannabis composed of at least three genes. The newly revealed Cannabis SP genes comprise CsSP-1, CsSP-2 and CsSP-3, encoded by genomic sequence as set forth in SEQ. ID. NO: 1, 4 and 7, coding sequence as set forth in SEQ. ID. NO: 2, 5 and 8, and amino acid sequence as set forth in SEQ. ID. NO: 3, 6 and 9, respectively. According to main aspects of the present invention, genome editing-targeted mutation in at least one of the aforementioned CsSP genes, which reduces the functional expression of the gene, affect the plant sympodial growth habit which plays a key role in determining plant architecture.

The term ‘SELF-PRUNING 5G’ or ‘SP5G’ in the context of the present invention refers to a gene encoding florigen paralog and flowering repressor responsible for loss of day-length-sensitive flowering. It is further acknowledged that SPSG expression is induced to high levels during long days in wild species. It is within the scope of the current invention that CRISPR/Cas9-engineered mutations in SPSG cause rapid flowering and enhance the compact determinate growth habit of Cannabis plants, resulting in a quick burst of flower production that turns to an early yield. The findings of the current invention suggest that variation in SP and/or SPSG facilitate the production of cultivated Cannabis strains with improved demonstration traits. The inventors of the present invention use gene editing techniques to rapidly improve yield traits in crop such as Cannabis.

The term “domestication trait” or agronomic trait are herein used interchangeably. In general, the initial phase of domestication represents a selection for increased adaptation to human cultivation, consumption, and utilization. Through multidisciplinary domestication research, a broad range of subjects is addressed, including the geographic and ecological origin of crop plants, the inheritance of domestication traits, the dispersal of crop plants from their centers of origin, and the timing and speed of domestication. With adaptation to changing human needs, domestication research has had a significant impact for continued crop improvement. It is herein acknowledged that plant domestication includes acquiring by the plants traits that make the plant worth of cultivation. These include traits that allow a crop to be reliably sown, cultivated and harvested, such as uniform seed germination, growth and fruit ripening. The domesticated plant is selected for improved qualities. Improved domestication traits within the scope of the present invention include, but are not limited to, reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

As used herein the term “genetic modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Cannabis plants with improved domestication traits are generated using genome editing mechanism. This technique enables to achieve in planta modification of specific genes that relate to and/or control the flowering time and plant architecture in the Cannabis plant.

The term “genome editing”, or “genome/genetic modification” or “genome engineering” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations. In the context of the present invention, the term also include base editing technique.

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

The term “base editing” or “base-editing” in the context of the present invention refers to a genome editing approach that uses components from CRISPR systems together with other enzymes to directly introduce point mutations into cellular DNA or RNA without making double-stranded DNA breaks (DSBs). It is within the scope that DNA base editors comprise a catalytically disabled or inactivated nuclease, called herein nickase (nCas) fused to a nucleobase deaminase enzyme or a DNA glycosylase inhibitor. It is acknowledged that RNA base editors achieve analogous changes using components that target RNA. According to aspects of the present invention, base editors directly convert one base or base pair into another, enabling the efficient introduction of specific and precise point mutations in non-dividing cells without generating excess undesired editing byproducts such as indels, translocations, and rearrangements derived from DSBs created by nucleases such as Cas9 or any other Cas.

It is further within the scope of the current invention that DNA base editors (BEs) comprise fusions between a catalytically impaired Cas nuclease and a base-modification enzyme that operates on single-stranded DNA (ssDNA) but not double-stranded DNA (dsDNA). Without wishing to be bound by theory, it is noted that upon binding to its target locus in DNA, base pairing between the guide RNA and target DNA strand leads to displacement of a small segment of single-stranded DNA in an “R-loop”. DNA bases within this single-stranded DNA bubble are modified by the deaminase enzyme. To improve efficiency in eukaryotic cells, the catalytically disabled nuclease also generates a nick in the non-edited DNA strand, inducing cells to repair the non-edited strand using the edited strand as a template.

It is within the scope of the present invention that two classes of DNA base editor have been described: cytosine base editors (CBEs) which convert a C·G base pair into a T·A base pair, and adenine base editors (ABEs) which convert an A·T base pair to a G·C base pair. Together, CBEs and ABEs can mediate all four possible transition mutations (C to T, A to G, T to C, and G to A). In RNA, targeted adenosine conversion to inosine has been used in both antisense and Cas13-guided RNA-targeting methods.

Reference is now made to exemplary genome editing terms used by the current disclosure:

Genome Editing Glossary

| | |
|---|---|
| Cas = CRISPR-associated genes | Indel = insertion and/or deletion |
| Cas9, Csn1 = a CRISPR-associated | NHEJ = Non-Homologous |
| protein | End Joining |
| containing two nuclease domains, | PAM = Protospacer-Adjacent Motif |
| that is programmed by small | RuvC = an endonuclease |
| RNAs to cleave DNA | domain named for |
| crRNA = CRISPR RNA | an E. coli protein involved |
| | in DNA repair |
| dCAS9 = nuclease-deficient Cas9 | sgRNA = single guide RNA |
| DSB = Double-Stranded Break | tracrRNA, trRNA = |
| gRNA = guide RNA | trans-activating crRNA |
| HDR = Homology-Directed Repair | TALEN = Transcription- |
| HNH = an endonuclease domain | Activator Like |
| named for characteristic histidine | Effector Nuclease |
| and asparagine residues | ZFN = Zinc-Finger Nuclease |

According to specific aspects of the present invention, the CRISPR (Clustered Regularly Interspaced Short Palindromic Repeats) and CRISPR-associated (Cas) genes are used for the first time for generating genome modification in targeted genes in the Cannabis plant. It is herein acknowledged that the functions of CRISPR (Clustered Regularly Interspaced Short Palindromic Repeats) and CRISPR-associated (Cas) genes are essential in adaptive immunity in select bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

According to further aspects of the invention, Cas protein, such as Cas9 (also known as Csnl) is required for gene silencing. Cas9 participates in the processing of crRNAs, and is responsible for the destruction of the target DNA. Cas9′s function in both of these steps relies on the presence of two nuclease domains, a RuvC-like nuclease domain located at the amino terminus and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand, while the RuvC domain cleaves the noncomplementary strand.

It is further noted that the double-stranded endonuclease activity of Cas9 also requires that a short conserved sequence, (2-5 nts) known as protospacer-associated motif (PAM), follows immediately 3′-of the crRNA complementary sequence.

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A, or Cas having a mutation resulting in a nickase Cas (nCas).

Reference is now made to an example of CRISPR/Cas9 mechanism of action as depicted by Xie, Kabin, and Yinong Yang. “RNA guided genome editing in plants using a CRISPR-Cas system.” Molecular plant 6.6 (2013): 1975-1983. As shown in this figure, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

It is within the scope of the present invention that the Cas gene may be selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1,Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpl1, Csb 1, Csb2, Csb3, Csx17, Csx14, Csx10, Csx16, CsaX, Csx3, Csz1, Csx15, Csf1, Csf2, Csf3, Csf4, and Cu1966, bacteriophages Cas such as CasΦ (Cas-phi), Cas 12f1, split Cas such as split Cas12a or split Cas9 or DlOA or mutation resulting in a nickase Cas (nCas) and any combination thereof.

It is further within the scope of the present invention that the gRNA or sgRNA sequence comprises a 3′ Protospacer Adjacent Motif (PAM) selected from the group consisting of NGG (SpCas), NNNNGATT (NmeCas9), NNAGAAW (StCas9), NAAAAC (TdCas9), NNGRRT (SaCas9), TTTR PAM (Casl2f1) and TBN (Cas-phi).

A guide nucleic acid includes gRNA, gDNA, crRNA and crDNA. The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

The term “Next-generation sequencing” or “NGS” as used herein refers hereinafter to massively, parallel, high- throughput or deep sequencing technology platforms that perform sequencing of millions of small fragments of DNA in parallel. Bioinformatics analyses are used to piece together these fragments by mapping the individual reads to the reference genome.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA or at the level of protein. It is within the scope of the present invention that the term gene knockdown also refers to a loss of function mutation and /or gene knockout mutation in which an organism's genes is made inoperative or nonfunctional.

The term “gene silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it.

The term “loss of function mutation” as used herein refers to a type of mutation in which the altered gene product lacks the function of the wild-type gene. A synonyms of the term included within the scope of the present invention is null mutation.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term ‘sympodial growth’ as used herein refers to a type of bifurcating branching pattern where one branch develops more strongly than the other, resulting in the stronger branches forming the primary shoot and the weaker branches appearing laterally. A sympodium, also referred to as a sympode or pseudaxis, is the primary shoot, comprising the stronger branches, formed during sympodial growth. In some aspects of the present invention, sympodial growth occurs when the apical meristem is terminated and growth is continued by one or more lateral meristems, which repeat the process. The apical meristem may be consumed to make an inflorescence or other determinate structure, or it may be aborted.

It is further within the scope of the current invention that the shoot section between two successive inflorescences is called the ‘sympodium’, and the number of leaf nodes per sympodium is referred to as the ‘sympodial index’ (spi). The first termination event activates the ‘sympodial cycle’. In sympodial plants, the apparent main shoot consists of a reiterated array of ‘sympodial units’. A mutant sp gene accelerates the termination of sympodial units but does not change the sympodial habit. The result is a progressive reduction in the number of vegetative nodes between inflorescences in a pattern that depends on light intensity and genetic background.

The term “earliness” refers hereinafter to early flowering and/or rapid transition from the vegetative to reproductive stages, or reduced ‘time to initiation of flowering’ and more generally to earlier completion of the life-cycle.

The term ‘reduced flowering time’ as used herein refers to time to production of first inflorescence. Such a trait can be evaluated or measured, for example, with reference to the number of leaves produced prior to appearance of the first inflorescence.

The term ‘harvest index’ can be herein defined as the total yield per plant weight.

The term ‘day length’ or ‘day length sensitivity’ as used in the context of the present invention generally refers to photoperiodism, which is the physiological reaction of organisms to the length of day or night. Photoperiodism can also be defined as the developmental responses of plants to the relative lengths of light and dark periods. Plants are classified under three groups according to the photoperiods: short-day plants, long-day plants, and day-neutral plants. Photoperiodism affects flowering by inducing the shoot to produce floral buds instead of leaves and lateral buds. It is within the scope of the present invention that Cannabis is included within the short-day facultative plants. The Cannabis plants of the present invention are genetically modified so as to exhibit loss of day-length sensitivity, which is highly desirable agronomical trait enabling enhanced yield of the cultivated crop.

The term ‘determinate’ or ‘determinate growth’ as used herein refers to plant growth in which the main stem ends in an inflorescence or other reproductive structure (e.g. a bud) and stops continuing to elongate indefinitely with only branches from the main stem having further and similarly restricted growth. It also refers to growth characterized by sequential flowering from the central or uppermost bud to the lateral or basal buds. It further means naturally self-limited growth, resulting in a plant of a definite maximum size.

The term ‘indeterminate’ or ‘indeterminate growth’ as used herein refers to plant growth in which the main stem continues to elongate indefinitely without being limited by a terminal inflorescence or other reproductive structure. It also refers to growth characterized by sequential flowering from the lateral or basal buds to the central or uppermost buds.

The term “orthologue” as used herein refers hereinafter to one of two or more homologous gene sequences found in different species.

The term “functional variant” or “functional variant of a nucleic acid or amino acid sequence” as used herein, for example with reference to SEQ ID NOs: 1, 4 or 7 refers to a variant of a sequence or part of a sequence which retains the biological function of the full non-variant allele (e.g. CsSP or CsSP5G allele) and hence has the activity of SP or SP5G expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those mat are identical or associated with a single gene or multiple genes or their products or even a gene disrupting or controlled by a genetic factor contributing to the phenotype represented by the locus.

According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele. In the context of the current invention, the term allele refers to the three identified SP genes in Cannabis, namely CsSP-1, CsSP-2 and CsSP-3 having the genomic nucleotide sequence as set forth in SEQ ID NOs: 1, 4 and 7, respectively. As well as to four identified SP5G genes in Cannabis, namely CsSP5G-1, CsSP5G-2, and CsSP5G-4 having the genomic nucleotide sequence as set forth in SEQ ID NOs: 10, 13, 18 and 19, respectively.

As used herein, the term “locus” (loci plural) means a specific place or places or region or a site on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

In specific embodiments, the Cannabis plants of the present invention comprise homozygous configuration of at least one of the mutated Cssp genes (i.e. Cssp-1, Cssp-2 and Cssp-3) and/or the mutated Cssp5g genes (i.e. Cssp5g-1, Cssp5g-2, Cssp5g-3 and Cssp5g-4).

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, S SEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three- dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a “modified” or a “mutant” plant is a plant that has been altered compared to the naturally occurring wild type (WT) plant. Specifically, the endogenous nucleic acid sequences of each of the SP or SP5G homologs in Cannabis (nucleic acid sequences CsSP-1, CsSP-2 and CsSP-3; and/or CsSP5G-1, CsSP5G-2, CsSP5G-3 and CsSP5G-4) have been altered compared to wild type sequences using mutagenesis and/or genome editing methods as described herein. This causes inactivation of the endogenous SP and/or SP5G genes and thus disables SP and/or SP5G function. Such plants have an altered phenotype and show improved domestication traits such as determinant plant architecture, synchronous and/or early flowering and loss of day length sensitivity compared to wild type plants. Therefore, the improved domestication phenotype is conferred by the presence of at least one mutated endogenous Cssp and /or Cssp5g gene in the Cannabis plant genome which has been specifically targeted using genome editing technique.

According to further aspects of the present invention, the at least one improved domestication trait is not conferred by the presence of transgenes expressed in Cannabis.

It should be noted that nucleic acid sequences of wild type alleles are designated using capital letters namely CsSP-1, CsMSP-2 and CsSP-3; and CsSP5G-1, CsMSP5G-2, CsSP5G-3 and CsSP5G-4. Mutant sp and sp5g nucleic acid sequences use non-capitalization. Cannabis plants of the invention are modified plants compared to wild type plants which comprise and express mutant Cssp and/or Cssp5g alleles.

It is further within the scope of the current invention that sp and/or sp5g mutations that down-regulate or disrupt functional expression of the wild-type SP and/or SP5G sequence respectively, may be recessive, such that they are complemented by expression of a wild-type sequence.

It is further noted that a wild type Cannabis plant is a plant that does not have any mutant sp and/or sp5g alleles.

Main aspects of the invention involve targeted mutagenesis methods, specifically genome editing, and exclude embodiments that are solely based on generating plants by traditional breeding methods. In a further embodiment of the current invention, as explained herein, the improved domestication at least one trait is not due to the presence of a transgene.

The inventors have generated mutant Cannabis lines with mutations inactivating at least one CsSP and/or CsSP5G homoeoallele which confer heritable improved domestication trait(s). In this way no functional CsSP and/or CsSP5G protein is made. Thus, the invention relates to these mutant Cannabis lines and related methods.

It is further within the scope of the present invention that breeding Cannabis cultivars with mutated sp allele enables the mechanical harvest of the plant. According to a further aspect of the present invention, loss of SP function results in compact Cannabis plants with reduced height, reduced number of sympodial units and determinate growth when compared with WT Cannabis.

According to a main aspect of the present invention, modifying Cannabis shoot architecture by selection for mutations in florigen flowering pathway genes allowed major improvements in plant architecture and yield. In particular, a mutation in the antiflorigen SELFPRUNING (SP) gene (sp classic) provided compact ‘determinate’ growth that translated to a burst of flowers, thereby enabling largescale field production.

According to one embodiment of the present invention, SELFPRUNING (SP) homologues and related florigen family members such as SELF-PRUNING 5G (SP5G) have been identified in both genome and transcriptome in Cannabis.

The work inter alfa described has important implications. The results have shown that CRISPR/Cas9 can be used to create heritable mutations in florigen pathway family members that result in desirable phenotypic effects.

To edit multiple domestication genes simultaneously and stack the resulting allelic variants, on option is that several gRNAs can be assembled to edit several genes into one construct, by using the Csy4 multi-gRNA system. The construct is then transformed via an appropriate vector into several wild-Cannabis accessions.

It is further within the scope of the current invention that Cannabis SP genes, namely CsSP-1, CsSP-2 and CsSP-3 having genomic nucleotide sequence as set forth in SEQ. ID. NO.: 1, 4 and 7 respectively, were targeted using guide RNAs as set forth in SEQ ID NO: 22-126, 127-211 and 212-283, respectively. Several mutated alleles have been identified. Notably, the plants with mutated sp alleles were more compact than the wild type plants lacking the mutated allele.

To identify other targets for plant architecture modification without negative effects on productivity, homologues of SELF-PRUNING 5G (SP5G) (Cs-SP5G), another florigen repressor, were identified in Cannabis. Cannabis SP5G genes, namely CsSP5G-1, CsSP5G-2, CsSP5G-3 and CsSP5G-4 having genomic nucleotide sequence as set forth in SEQ. ID. NO.: 10, 13, 16 and 19 respectively, were targeted using guide RNAs as set forth in SEQ ID NO: 284-516, 517-745, 746-828 and 829-916, respectively. Several mutated alleles have been identified.

It is herein acknowledged that SP5G represses flowering predominantly in primary and canonical axillary shoots.

According to one embodiment of the present invention, SP5G was shown to control primary and canonical axillary shoot flowering time and to contribute to controlling day-length sensitivity. In other words, reduced SP5G activity was shown to mitigate day-length sensitivity.

According to a further embodiment of the present invention, CRISPR-Cas9-induced null sp5g mutations in Cannabis eliminate day-length sensitivity and causing faster primary and axillary shoot flowering.

The present invention discloses that the combination of both mutations sp5g and sp results in faster-flowering Cannabis plants with typical sp determinate growth. Such Cannabis plants could have agronomic value, particularly for the desirable trait of earliness of yield.

According to specific aspects of the present invention, Cs-sp5g/ sp double mutants are not simply additive but substantially more compact than mutant sp Cannabis plants owing to faster axillary shoot flowering and earlier termination of sympodial cycling.

According to further aspects of the present invention, compared with wild type and/or sp Cannabis plants, Cs-sp5g/sp double mutant plants provide a more rapid flowering burst, and reach final harvest sooner.

It is further within the scope of the present invention that the harvest index (defined as the total yield per plant weight) of the Cs-sp5g/ sp double mutant plants is higher than that for wild type and/or sp mutant Cannabis plants.

According to a further specific aspect of the present invention, large-scale Cannabis production based on sp determinate growth may be achieved only in the absence of day-length sensitivity, i.e. by loss of function mutation in at least one of Cssp5g-1, Cssp5g-2, Cssp5g-3 or Cssp5g-4 as set forth in SEQ ID NO.: 10, 13 16 and 19, respectively.

It is further within the scope of the present invention that targeting SP5G homologs and/or other diurnally regulated CENTRORADIALIS/TERMINAL FLOWER 1/SELF-PRUNING (CETS) genes may allow immediate customization of day-length sensitivity in Cannabis elite germplasm to expand the geographical range of cultivation, and could serve as a first step toward engineering the domestication of wild Cannabis species with agricultural potential.

The loss of function mutation may be a deletion or insertion (“indels”) with reference the wild type CsSP and/or CsSP5G allele sequence. The deletion may comprise 1-20 or more nucleotides, for example 1, 2, 3, 4, 5, 6, 7, 8, 9, 10, 1, 12, 13, 14, 15, 16, 17, 18 or 20 nucleotides or more in one or more strand. The insertion may comprise 1-20 or more nucleotides, for example 1, 2, 3, 4, 5, 6, 7, 8, 9, 10, 1, 12, 13, 14, 15, 16, 17, 18 or 20 or more nucleotides in one or more strand.

The plant of the invention includes plants wherein the plant is heterozygous for the each of the mutations. In a preferred embodiment however, the plant is homozygous for the mutations. Progeny that is also homozygous can be generated from these plants according to methods known in the art.

It is further within the scope that variants of a particular CsSP and/or CsSP5G nucleotide or amino acid sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant CsSP and/or CsSP5G nucleotide sequence of the CsSP and/or CsSP5G allele as shown in SEQ ID NO 1, 4 or 7; and/or SEQ ID NO 10, 13, 18 or 19, respectively. Sequence alignment programs to determine sequence identity are well known in the art.

Also, the various aspects of the invention encompass not only a CsSP and/or CsSP5G nucleic acid sequence or amino acid sequence, but also fragments thereof. By “fragment” is intended a portion of the nucleotide sequence or a portion of the amino acid sequence and hence of the protein encoded thereby. Fragments of a nucleotide sequence may encode protein fragments that retain the biological activity of the native protein, in this case improved domestication trait.

According to a further embodiment of the invention, the herein newly identified Cannabis SP and/or SPSG locus (CsSP and/or CsSP5G) have been targeted using the double sgRNA strategy.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein- RNP's) in order to bypass the need for in vivo transcription and translation of the Cas9+gRNA plasmid in planta to achieve gene editing.

It is also possible to create a genome edited plant and use it as a rootstock. Then, the Cas protein and gRNA can be transported via the vasculature system to the top of the plant and create the genome editing event in the scion .

It is within the scope of the present invention that the usage of CRISPR/Cas system for the generation of Cannabis plants with at least one improved domestication trait, allows the modification of predetermined specific DNA sequences without introducing foreign DNA into the genome by GMO techniques. According to one embodiment of the present invention, this is achieved by combining the Cas nuclease (e.g. Cas9, Cpfl and the like) with a predefined guide RNA molecule (gRNA). The gRNA is complementary to a specific DNA sequence targeted for editing in the plant genome and which guides the Cas nuclease to a specific nucleotide sequence. The predefined gene specific gRNA's are cloned into the same plasmid as the Cas gene and this plasmid is inserted into plant cells. Insertion of the aforementioned plasmid DNA can be done, but not limited to, using different delivery systems, biological and/or mechanical, e.g. Agrobacterium infiltration, virus based plasmids for delivery of the genome editing molecules and mechanical insertion of DNA (PEG mediated DNA transformation, biolistics, etc.).

It is further within the scope of the present invention that upon reaching the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually create a mutation at the cleavage site. For example, it is acknowledged that a deletion form of the mutation consists of at least 1 base pair deletion. As a result of this base pair deletion the gene coding sequence is disrupted and the translation of the encoded protein is compromised either by a premature stop codon or disruption of a functional or structural property of the protein. Thus DNA is cut by the Cas9 protein and re-assembled by the cell's DNA repair mechanism.

It is further within the scope that improved domestication traits in Cannabis plants is herein produced by generating gRNA with homology to a specific site of predetermined genes in the Cannabis genome i.e. SP and/or SP5G genes, sub cloning this gRNA into a plasmid containing the Cas9 gene, and insertion of the plasmid into the Cannabis plant cells. In this way site specific mutations in the SP and/or SP5G genes are generated thus effectively creating non-active molecules, resulting in loss of day length sensitivity, reduced flowering time and determinant growth habit of the genome edited plant.

According to one embodiment of the present invention, a modified Cannabis plant exhibiting at least one improved domestication trait compared with wild type Cannabis is disclosed. The modified plant comprises at least one mutated Cannabis SELF PRUNING (SP) (CsSP) gene and/or at least one mutated Cannabis SELF PRUNING 5G (SP5G) (CsSP5G) gene.

According to another embodiment of the present invention, the SELF PRUNING (SP) Cannabis gene is selected from the group consisting of CsSP-1 having a genomic nucleotide sequence as set forth in SEQ ID NO: 1 or a functional variant thereof, CsSP-2 having a genomic nucleotide sequence as set forth in SEQ ID NO: 4 or a functional variant thereof, CsSP-3 having a genomic nucleotide sequence as set forth in SEQ ID NO: 7 or a functional variant thereof and any combination thereof.

According to another embodiment of the present invention, said SELF PRUNING 5G (SP5G) Cannabis gene is selected from the group consisting of CsSP5G-1 having a genomic nucleotide sequence as set forth in SEQ ID NO: 10 or a functional variant thereof, CsSP5G-2 having a genomic nucleotide sequence as set forth in SEQ ID NO: 13 or a functional variant thereof, CsSP5G-3 having a genomic nucleotide sequence as set forth in SEQ ID NO: 16 or a functional variant thereof, CsSP5G-4 having a genomic nucleotide sequence as set forth in SEQ ID NO: 19 or a functional variant thereof and any combination thereof.

According to another embodiment of the present invention, said functional variant has at least 75% sequence identity to said CsSP or said CsSP5G nucleotide sequence.

According to another embodiment of the present invention, said mutation is introduced using mutagenesis, small interfering RNA (siRNA), microRNA (miRNA), artificial miRNA (amiRNA), DNA introgression, endonucleases or any combination thereof.

According to another embodiment of the present invention, said mutation is introduced using targeted genome modification. According to another embodiment of the present invention, said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

According to another embodiment of the present invention, said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1,Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpl1, Csb1, Csb2, Csb3, Csx17, Csx14, Csx10, Csx16, CsaX, Csx3, Csz1, Csx15, Csf1, Csf2, Csf3, Csf4, and Cu1966 and any combination thereof.

According to another embodiment of the present invention, the mutated CsSP or CsSP5G gene is a CRISPR/Cas9- induced heritable mutated allele.

According to another embodiment of the present invention, said mutation is a missense mutation, nonsense mutation, insertion, deletion, indel, substitution or duplication.

According to another embodiment of the present invention, the insertion or the deletion produces a gene comprising a frameshift.

According to another embodiment of the present invention, said plant is homozygous for said at list one CsSP mutated gene.

According to another embodiment of the present invention, said plant is homozygous for said at list one CsSP5G mutated gene.

According to another embodiment of the present invention, said plant is a Cssp Cssp5g double mutant.

According to another embodiment of the present invention, said mutation is in the coding region of said allele, a mutation in the regulatory region of said allele, or an epigenetic factor.

According to another embodiment of the present invention, said mutation is a silencing mutation, a knockdown mutation, a knockout mutation, a loss of function mutation or any combination thereof.

According to another embodiment of the present invention, said mutation is generated in planta.

According to another embodiment of the present invention, said mutation is generated in planta via introduction of a construct comprising (a) Cas DNA and gRNA sequence selected from the group consisting of SEQ ID NO: 22-SEQ ID NO: 916 and any combination thereof, or (b) a ribonucleoprotein (RNP) complex comprising Cas protein and gRNA sequence selected from the group consisting of SEQ ID NO: 22-916 and any combination thereof.

According to another embodiment of the present invention, said mutation in said CsSP-1 is generated in planta via introduction of a construct comprising (a) Cas DNA and gRNA sequence selected from the group consisting of SEQ ID NO: 22-SEQ ID NO: 126 and any combination thereof, or (b) a ribonucleoprotein (RNP) complex comprising Cas protein and gRNA sequence selected from the group consisting of SEQ ID NO: 22-126 and any combination thereof.

According to another embodiment of the present invention, said mutation in said CsSP-2 is generated in planta via introduction of a construct comprising (a) Cas DNA and gRNA sequence selected from the group consisting of SEQ ID NO: 127-SEQ ID NO: 211 and any combination thereof, or (b) a ribonucleoprotein (RNP) complex comprising Cas protein and gRNA sequence selected from the group consisting of SEQ ID NO: 127-211 and any combination thereof.

According to another embodiment of the present invention, said mutation in said CsSP-3 is generated in planta via introduction of a construct comprising (a) Cas DNA and gRNA sequence selected from the group consisting of SEQ ID NO: 212-SEQ ID NO: 283 and any combination thereof, or (b) a ribonucleoprotein (RNP) complex comprising Cas protein and gRNA sequence selected from the group consisting of SEQ ID NO: 212-283 and any combination thereof.

According to another embodiment of the present invention, said mutation in said CsSP5G-1 is generated in planta via introduction of a construct comprising (a) Cas DNA and gRNA sequence selected from the group consisting of SEQ ID NO: 284-SEQ ID NO: 516 and any combination thereof, or (b) a ribonucleoprotein (RNP) complex comprising Cas protein and gRNA sequence selected from the group consisting of SEQ ID NO: 284-516 and any combination thereof.

According to another embodiment of the present invention, said mutation in said CsSP5G-2 is generated in planta via introduction of a construct comprising (a) Cas DNA and gRNA sequence selected from the group consisting of SEQ ID NO: 517-SEQ ID NO: 745 and any combination thereof, or (b) a ribonucleoprotein (RNP) complex comprising Cas protein and gRNA sequence selected from the group consisting of SEQ ID NO: 517-745 and any combination thereof.

According to another embodiment of the present invention, said mutation in said CsSP5G-3 is generated in planta via introduction of a construct comprising (a) Cas DNA and gRNA sequence selected from the group consisting of SEQ ID NO: 746-SEQ ID NO: 828 and any combination thereof, or (b) a ribonucleoprotein (RNP) complex comprising Cas protein and gRNA sequence selected from the group consisting of SEQ ID NO: 746-828 and any combination thereof.

According to another embodiment of the present invention, said mutation in said CsSP5G-4 is generated in planta via introduction of a construct comprising (a) Cas DNA and gRNA sequence selected from the group consisting of SEQ ID NO: 829-SEQ ID NO: 916 and any combination thereof, or (b) a ribonucleoprotein (RNP) complex comprising Cas protein and gRNA sequence selected from the group consisting of SEQ ID NO: 829-916 and any combination thereof.

According to another embodiment of the present invention, said gRNA sequence comprises a 3′ NGG Protospacer Adjacent Motif (PAM).

According to another embodiment of the present invention, said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

According to another embodiment of the present invention, said plant has decreased expression levels of at least one of said CsSP genes.

According to another embodiment of the present invention, the sequence of said expressed CsSP gene is selected from the group consisting of: SEQ ID NO: 2, SEQ ID NO: 3, SEQ ID NO: 5, SEQ ID NO: 6, SEQ ID NO: 8 and SEQ ID NO: 9 or a functional variant thereof.

According to another embodiment of the present invention, said plant has decreased expression levels of at least one of said CsSP5G genes.

According to another embodiment of the present invention, the sequence of said expressed CsSP5G gene is selected from the group consisting of: SEQ ID NO: 11, SEQ ID NO: 12, SEQ ID NO: 14, SEQ ID NO: 15, SEQ ID NO: 17, SEQ ID NO: 18, SEQ ID NO: 20 and SEQ ID NO: 21 or a functional variant thereof.

According to another embodiment of the present invention, said plant is semi-determinant.

According to another embodiment of the present invention, said plant has determinant growth habit.

According to another embodiment of the present invention, said plant flowers earlier than a corresponding wild type cannabis plant.

According to another embodiment of the present invention, said plant exhibits improved earliness as compared to a corresponding wild type cannabis plant.

According to another embodiment of the present invention, said plant exhibits suppressed sympodial shoot termination as compared to a corresponding wild type cannabis plant.

According to another embodiment of the present invention, said plant exhibits similar sympodial shoot termination as compared to a corresponding wild type cannabis plant.

According to another embodiment of the present invention, said plant exhibits suppressed or reduced day-length sensitivity as compared to a corresponding wild type cannabis plant.

According to another embodiment of the present invention, said Cannabis plant is selected from the group of species that includes, but is not limited to, Cannabis sativa (C. sativa), C. indica, C. ruderalis and any hybrid or cultivated variety of the genus Cannabis.

According to another embodiment of the present invention, said domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

According to another embodiment of the present invention, said Cssp Cssp5g double mutant is characterized by having a more than additive effect on a trait selected from the group consisting of compactness, earlier axillary shoot flowering, earlier termination of sympodial cycling, harvest index and any combination thereof as compared to wild type and/or sp mutant Cannabis plants.

It is further within the scope of the present invention to disclose a modified Cannabis plant, plant part or plant cell as defined in any of the above, wherein said plant does not comprise a transgene.

It is further within the scope of the present invention to disclose a plant part, plant cell or plant seed of a plant as defined in any of the above.

It is further within the scope of the present invention to disclose a tissue culture of regenerable cells, protoplasts or callus obtained from the modified Cannabis plant as defined in any of the above.

It is further within the scope of the present invention to disclose a method for producing a modified Cannabis plant exhibiting at least one improved domestication trait compared with wild type Cannabis, said method comprises steps of genetically modifying at least one Cannabis SELF PRUNING (SP) (CsSP) gene and/or at least one Cannabis SELF PRUNING 5G (SP5G) (CsSP5G) gene.

It is further within the scope of the present invention to disclose a method for producing a modified Cannabis plant exhibiting at least one improved domestication trait compared with wild type

Cannabis by targeted genome modification, said method comprises steps of genetically introducing a loss of function mutation in at least one Cannabis SELF PRUNING (SP) (CsSP) gene and/or at least one Cannabis SELF PRUNING 5G (SP5G) (CsSP5G) gene.

It is further within the scope of the present invention to disclose a method of improving at least one domestication trait compared with wild type Cannabis, comprising steps of producing a modified Cannabis plant, seed or plant part thereof, that is homozygous for at least one mutated CsSP5G gene in an sp background and enabling growth of said Cannabis plant, seed or plant part thereof.

It is further within the scope of the present invention, wherein said method comprises steps of: (a) identifying at least one Cannabis SP (CsSP) and/or at least one Cannabis SP5G (CsSP5G) allele; (b) synthetizing at least one guide RNA (gRNA) comprising a nucleotide sequence complementary to said at least one identified CsSP and/or CsSP5G allele; (c) transforming Cannabis plant cells with a construct comprising (a) Cas nucleotide sequence operably linked to said at least one gRNA, or (b) a ribonucleoprotein (RNP) complex comprising Cas protein and said at least one gRNA; (d) screening the genome of said transformed plant cells for induced targeted loss of function mutation in at least one of said CsSP and/or CsSP5G allele; (e) regenerating Cannabis plants carrying said loss of function mutation in at least one of said CsSP and/or CsSP5G allele; and (f) screening said regenerated plants for a Cannabis plant with improved domestication trait.

It is further within the scope of the present invention, wherein said step of screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one of said CsSP and/or CsSP5G allele.

It is further within the scope of the present invention, wherein said SELF PRUNING (SP) Cannabis gene is selected from the group consisting of CsSP-1 having a genomic nucleotide sequence as set forth in SEQ ID NO: 1 or a functional variant thereof, CsSP-2 having a genomic nucleotide sequence as set forth in SEQ ID NO: 4 or a functional variant thereof, CsSP-3 having a genomic nucleotide sequence as set forth in SEQ ID NO: 7 or a functional variant thereof and any combination thereof.

It is further within the scope of the present invention, wherein said SELF PRUNING 5G (SP5G) Cannabis gene is selected from the group consisting of CsSP5G-1 having a genomic nucleotide sequence as set forth in SEQ ID NO: 10 or a functional variant thereof, CsSP5G-2 having a genomic nucleotide sequence as set forth in SEQ ID NO: 13 or a functional variant thereof, CsSP5G-3 having a genomic nucleotide sequence as set forth in SEQ ID NO: 16 or a functional variant thereof, CsSP5G-4 having a genomic nucleotide sequence as set forth in SEQ ID NO: 19 or a functional variant thereof and any combination thereof.

It is further within the scope of the present invention, wherein said functional variant has at least 75% sequence identity to said CsSP or said CsSP5G nucleotide sequence.

It is further within the scope of the present invention, wherein said mutation is introduced using mutagenesis, small interfering RNA (siRNA), microRNA (miRNA), artificial miRNA (amiRNA), DNA introgression, endonucleases or any combination thereof.

It is further within the scope of the present invention, wherein said mutation is introduced using targeted genome modification.

It is further within the scope of the present invention, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is further within the scope of the present invention, wherein said Cas gene is selected from the group consisting of Cas3, Cas4, Cas5, Cas5e (or CasD), Cas6, Cas6e, Cas6f, Cas7, Cas8a1, Cas8a2, Cas8b, Cas8c, Cas9, Cas10, Cast10d, Cas12, Cas13, Cas14, CasX, CasF, CasG, CasH, Csy1,Csy2, Csy3, Cse1 (or CasA), Cse2 (or CasB), Cse3 (or CasE), Cse4 (or CasC), Csc1, Csc2, Csa5, Csn1, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csz1, Csx15, Csf1, Csf2, Csf3, Csf4, and Cu1966 and any combination thereof.

It is further within the scope of the present invention, wherein the mutated CsSP or CsSP5G gene is a CRISPR/Cas9- induced heritable mutated allele.

It is further within the scope of the present invention, wherein said mutation is a missense mutation, nonsense mutation, insertion, deletion, indel, substitution or duplication.

It is further within the scope of the present invention, wherein the insertion or the deletion produces a gene comprising a frameshift.

It is further within the scope of the present invention, wherein said plant is homozygous for said at list one CsSP mutated gene.

It is further within the scope of the present invention, wherein said plant is homozygous for said at list one CsSP5G mutated gene.

It is further within the scope of the present invention, wherein said plant is a Cssp Cssp5g double mutant.

It is further within the scope of the present invention, wherein said mutation is in the coding region of said allele, a mutation in the regulatory region of said allele, or an epigenetic factor.

It is further within the scope of the present invention, wherein said mutation is a silencing mutation, a knockdown mutation, a knockout mutation, a loss of function mutation or any combination thereof.

It is further within the scope of the present invention, wherein said mutation is generated in planta.

It is further within the scope of the present invention, wherein said mutation is generated in planta via introduction of a construct comprising (a) Cas DNA and gRNA sequence selected from the group consisting of SEQ ID NO: 22-SEQ ID NO: 916 and any combination thereof, or (b) a ribonucleoprotein (RNP) complex comprising Cas protein and gRNA sequence selected from the group consisting of SEQ ID NO: 22-916 and any combination thereof.

It is further within the scope of the present invention, wherein said mutation in said CsSP-1 is generated in planta via introduction of a construct comprising (a) Cas DNA and gRNA sequence selected from the group consisting of SEQ ID NO: 22-SEQ ID NO: 126 and any combination thereof, or (b) a ribonucleoprotein (RNP) complex comprising Cas protein and gRNA sequence selected from the group consisting of SEQ ID NO: 22-126 and any combination thereof.

It is further within the scope of the present invention, wherein said mutation in said CsSP-2 is generated in planta via introduction of a construct comprising (a) Cas DNA and gRNA sequence selected from the group consisting of SEQ ID NO: 127-SEQ ID NO: 211 and any combination thereof, or (b) a ribonucleoprotein (RNP) complex comprising Cas protein and gRNA sequence selected from the group consisting of SEQ ID NO: 127-211 and any combination thereof.

It is another object of the present invention to disclose the method as defined in any of the above, wherein said mutation in said CsSP-3 is generated in planta via introduction of a construct comprising (a) Cas DNA and gRNA sequence selected from the group consisting of SEQ ID NO: 212-SEQ ID NO: 283 and any combination thereof, or (b) a ribonucleoprotein (RNP) complex comprising Cas protein and gRNA sequence selected from the group consisting of SEQ ID NO: 212-283 and any combination thereof.

It is further within the scope of the present invention, wherein said mutation in said CsSP5G-1 is generated in planta via introduction of a construct comprising (a) Cas DNA and gRNA sequence selected from the group consisting of SEQ ID NO: 284-SEQ ID NO: 516 and any combination thereof, or (b) a ribonucleoprotein (RNP) complex comprising Cas protein and gRNA sequence selected from the group consisting of SEQ ID NO: 284-516 and any combination thereof.

It is further within the scope of the present invention, wherein said mutation in said CsSP5G-2 is generated in planta via introduction of a construct comprising (a) Cas DNA and gRNA sequence selected from the group consisting of SEQ ID NO: 517-SEQ ID NO: 745 and any combination thereof, or (b) a ribonucleoprotein (RNP) complex comprising Cas protein and gRNA sequence selected from the group consisting of SEQ ID NO: 517-745 and any combination thereof.

It is further within the scope of the present invention, wherein said mutation in said CsSP5G-3 is generated in planta via introduction of a construct comprising (a) Cas DNA and gRNA sequence selected from the group consisting of SEQ ID NO: 746-SEQ ID NO: 828 and any combination thereof, or (b) a ribonucleoprotein (RNP) complex comprising Cas protein and gRNA sequence selected from the group consisting of SEQ ID NO: 746-828 and any combination thereof.

It is further within the scope of the present invention, wherein said mutation in said CsSP5G-4 is generated in planta via introduction of a construct comprising (a) Cas DNA and gRNA sequence selected from the group consisting of SEQ ID NO: 829-SEQ ID NO: 916 and any combination thereof, or (b) a ribonucleoprotein (RNP) complex comprising Cas protein and gRNA sequence selected from the group consisting of SEQ ID NO: 829-916 and any combination thereof.

It is further within the scope of the present invention, wherein said gRNA sequence comprises a 3′ NGG Protospacer Adjacent Motif (PAM).

It is further within the scope of the present invention, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

It is further within the scope of the present invention, wherein said plant has decreased expression levels of at least one of said CsSP genes.

It is further within the scope of the present invention, wherein the sequence of said expressed CsSP gene is selected from the group consisting of: SEQ ID NO: 2, SEQ ID NO: 3, SEQ ID NO: 5, SEQ ID NO: 6, SEQ ID NO: 8 and SEQ ID NO: 9 or a functional variant thereof.

It is further within the scope of the present invention, wherein said plant has decreased expression levels of at least one of said CsSP5G genes.

It is further within the scope of the present invention, wherein the sequence of said expressed CsSP5G gene is selected from the group consisting of: SEQ ID NO: 11, SEQ ID NO: 12, SEQ ID NO: 14, SEQ ID NO: 15, SEQ ID NO: 17, SEQ ID NO: 18, SEQ ID NO: 20 and SEQ ID NO: 21 or a functional variant thereof.

It is further within the scope of the present invention, wherein said plant is semi-determinant.

It is further within the scope of the present invention, wherein said plant has determinant growth habit.

It is further within the scope to disclose the method as defined in any of the above, wherein said plant flowers earlier than a corresponding wild type cannabis plant.

It is further within the scope to disclose the method as defined in any of the above, wherein said plant exhibits improved earliness as compared to a corresponding wild type cannabis plant.

It is further within the scope to disclose the method as defined in any of the above, wherein said plant exhibits suppressed sympodial shoot termination as compared to a corresponding wild type cannabis plant.

It is further within the scope to disclose the method as defined in any of the above, wherein said plant exhibits similar sympodial shoot termination as compared to a corresponding wild type cannabis plant.

It is further within the scope to disclose the method as defined in any of the above, wherein said plant exhibits suppressed or reduced day-length sensitivity as compared to a corresponding wild type cannabis plant.

It is further within the scope to disclose the method as defined in any of the above, wherein said Cannabis plant is selected from the group of species that includes, but is not limited to, Cannabis sativa (C. sativa), C. indica, C. ruderalis and any hybrid or cultivated variety of the genus Cannabis.

It is further within the scope to disclose a modified Cannabis plant, plant part or plant cell produced by the method as defined in any of the above, wherein said plant does not comprise a transgene.

It is further within the scope to disclose a plant part, plant cell or plant seed of a plant produced by the method as defined in any of the above.

It is further within the scope to disclose a tissue culture of regenerable cells, protoplasts or callus obtained from the modified Cannabis plant produced by the method as defined in any of the above.

It is further within the scope to disclose the method as defined in any of the above, wherein said at least one domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

It is further within the scope to disclose the method as defined in any of the above, wherein said Cssp Cssp5g double mutant is characterized by having a more than additive effect on a trait selected from the group consisting of compactness, earlier axillary shoot flowering, earlier termination of sympodial cycling, harvest index and any combination thereof as compared to wild type and/or sp mutant Cannabis plants.

It is further within the scope to disclose an isolated nucleotide sequence having at least 75% sequence identity to a CsSP genomic nucleotide sequence selected from the group consisting of SEQ ID NO: 1, SEQ ID NO: 4 and SEQ ID NO: 7.

It is further within the scope to disclose an isolated nucleotide sequence having at least 75% sequence identity to a CsSP5G genomic nucleotide sequence selected from the group consisting of SEQ ID NO: 10, SEQ ID NO: 13, SEQ ID NO: 16 and SEQ ID NO: 19.

It is further within the scope to disclose an isolated nucleotide sequence having at least 75% sequence identity to a CsSP nucleotide coding sequence selected from the group consisting of SEQ ID NO: 2, SEQ ID NO: 5 and SEQ ID NO: 8.

It is further within the scope to disclose an isolated nucleotide sequence having at least 75% sequence identity to a CsSP5G nucleotide coding sequence selected from the group consisting of SEQ ID NO: 11, SEQ ID NO: 14, SEQ ID NO: 17 and SEQ ID NO: 20.

It is further within the scope to disclose an isolated amino acid sequence having at least 75% sequence similarity to a CsSP amino acid sequence selected from the group consisting of SEQ ID NO: 3, SEQ ID NO: 6 and SEQ ID NO: 9.

It is further within the scope to disclose an isolated amino acid sequence having at least 75% sequence similarity to a CsSP5G amino acid sequence selected from the group consisting of SEQ ID NO: 12, SEQ ID NO: 15, SEQ ID NO: 18 and SEQ ID NO: 21.

It is further within the scope to disclose an isolated nucleotide sequence having at least 75% sequence identity to a CsSP-targeted gRNA nucleotide sequence as set forth in SEQ ID NO: 22-283.

It is further within the scope to disclose an isolated nucleotide sequence having at least 75% sequence identity to a CsSP5G-targeted gRNA nucleotide sequence as set forth in SEQ ID NO: 284-916.

It is further within the scope to disclose use of a nucleotide sequence as set forth in at least one of SEQ ID NO: 22-126 and any combination thereof for targeted genome modification of Cannabis SP-1 (CsSP-1) allele.

It is further within the scope to disclose use of a nucleotide sequence as set forth in at least one of SEQ ID NO: 127-211 and any combination thereof for targeted genome modification of Cannabis SP-2 (CsSP-2) allele.

It is further within the scope to disclose use of a nucleotide sequence as set forth in at least one of SEQ ID NO: 212-283 and any combination thereof for targeted genome modification of Cannabis SP-3 (CsSP-3) allele.

It is further within the scope to disclose use of a nucleotide sequence as set forth in at least one of SEQ ID NO: 284-516 and any combination thereof for targeted genome modification of Cannabis SP5G-1 (CsSP5G-1) allele.

It is further within the scope to disclose use of a nucleotide sequence as set forth in at least one of SEQ ID NO: 517-745 and any combination thereof for targeted genome modification of Cannabis SP5G-2 (CsSP5G-2) allele.

It is further within the scope to disclose use of a nucleotide sequence as set forth in at least one of SEQ ID NO: 746-828 and any combination thereof for targeted genome modification of Cannabis SP5G-3 (CsSP5G-3) allele.

It is further within the scope to disclose use of a nucleotide sequence as set forth in at least one of SEQ ID NO: 829-916 and any combination thereof for targeted genome modification of Cannabis SP5G-4 (CsSP5G-4) allele.

It is a further within the scope of the present invention to disclose a method for down regulation of Cannabis SP-1 (CsSP-1) gene, which comprises utilizing the nucleotide sequence as set forth in at least one of SEQ ID NO: 102, SEQ ID NO: 109, SEQ ID NO: 112, or a complementary sequence thereof, and a combination thereof, for introducing a loss of function mutation into said CsSP-1 gene using targeted genome editing.

It is a further within the scope of the present invention to disclose an isolated polynucleotide sequence having at least 80% sequence similarity to a polynucleotide sequence selected from the group consisting of SEQ ID NO: 1, SEQ ID NO: 918-922, SEQ ID NO: 924-925, SEQ ID NO: 927-933, SEQ ID NO: 935-938, or a complementary sequence thereof, and a combination thereof.

It is a further within the scope of the present invention to disclose use of a nucleotide sequence having at least 80% sequence similarity to a polynucleotide sequence selected from the group consisting of SEQ ID NO: 918-922, SEQ ID NO: 924-925, SEQ ID NO: 927-933, SEQ ID NO: 935-938, or a complementary sequence thereof, and a combination thereof for generating, identifying and/or screening for a Cannabis plant comprising within its genome mutant Cssp-1 allele.

It is a further within the scope of the present invention to disclose the use as defined in any of the above, wherein the presence of at least one polynucleotide sequence selected from the group consisting of a sequence having at least 80% similarity to SEQ ID NO: 1, SEQ ID NO: 917, SEQ ID NO: 923, SEQ ID NO: 926, SEQ ID NO: 934 or a complementary sequence thereof, or any combination thereof, indicates that the Cannabis plant comprises a wild type CsSP-1 allele, and the presence of at least one polynucleotide sequence selected from the group consisting of a sequence having at least 80% similarity to SEQ ID NO: 918-922, SEQ ID NO: 924-925, SEQ ID NO: 927-933, SEQ ID NO: 935-938, or a complementary sequence thereof, and a combination thereof, indicates that the Cannabis plant comprises a mutant Cssp-1 allele.

It is a further within the scope of the present invention to disclose use of a polynucleotide sequence having at least 80% similarity to SEQ ID NO: 102, SEQ ID NO: 109 and SEQ ID NO: 112, or a complementary sequence or any combination thereof, for targeted genome editing of Cannabis SP-1 (CsSP-1) gene.

It is a further within the scope of the present invention to disclose a detection kit for determining the presence or absence of a mutant Cssp-1 allele in a Cannabis plant, comprising a polynucleotide fragment having at least 80% similarity to SEQ ID NO: 918-922, SEQ ID NO: 924-925, SEQ ID NO: 927-933, SEQ ID NO: 935-938, or a complementary sequence thereof, and any combination thereof.

It is a further within the scope of the present invention to disclose the kit as defined in any of the above, wherein said kit is useful for identifying a Cannabis plant with improved domestication trait.

In order to understand the invention and to see how it may be implemented in practice, a plurality of preferred embodiments will now be described, by way of non-limiting example only, with reference to the following examples.

### EXAMPLE 1

Production of Cannabis plants with improved domestication traits by targeted genome editing

This example describes a generalized scheme of the process for generating the genome edited Cannabis plants of the present invention, The process comprises the following steps:
1. Designing and synthesizing gRNA's corresponding to a sequence targeted for editing. Editing event should be designed flanking with a unique restriction site sequence to allow easier screening of successful editing.
2. Carrying transformation using Agrobactedum or biolistics. For Agrobacterium and bioloistics transformation using a DNA plasmid, a vector containing a selection marker, Cas9 gene and relevant gRNA's is constructed. For biolistics using Ribonucleoprotein (RNP) complexes, RNP complexes are created by mixing the Cas9 protein with relevant gRNA's.
3. Performing regeneration in tissue culture. For DNA transformation, using antibiotics for selection of positive transformants.
4. Selecting positive transformants. Once regenerated plants appear in the regenerated tissue culture, obtaining leaf (or any other selected tissue) samples, extracting DNA from the obtained sample and preforming PCR. using primers flanking the editing region. The resulted PCR products are digested with enzymes recognizing the restriction site near original gRNA sequence. If editing event occurred, the restriction site will be disrupted and PCR product will not be cleaved. Absence of an editing event will result in a cleaved PCR product.

Production of Cannabis lines with mutated sp and/or sp5g gene may be achieved by at least one of the following breeding/cultivation schemes:

Scheme 1:
line stabilization by self pollination
Generation of F6 parental lines
Genome editing of parental lines
Crossing edited parental lines to generate an F1 hybrid plant

Scheme 2:
Identifying genes/alleles of interest
Designing gRNA
Transformation of plants with Cas9+gRNA constructs
Screening and identifying editing events
Genome editing of parental lines

It is noted that line stabilization may be performed by the following:
Induction of male flowering on female (XX) plants
Self pollination

According to some embodiments of the present invention, line stabilization requires about 6 self-crossing (6 generations) and done through a single seed descent (SSD) approach.

F1 hybrid seed production: Novel hybrids are produced by crosses between different Cannabis strains.

According to a further aspect of the current invention, shortening line stabilization is performed by Doubled Haploids (DH). More specifically, the CRISPR-Cas9 system is transformed into microspores to achieve DH homozygous parental lines. A doubled haploid (DH) is a genotype formed when haploid cells undergo chromosome doubling. Artificial production of doubled haploids is important in plant breeding. It is herein acknowledged that conventional inbreeding procedures take about six generations to achieve approximately complete homozygosity, whereas doubled haploidy achieves it in one generation.

It is within the scope of the current invention that genetic markers specific for Cannabis are developed and provided by the current invention:
Sex markers—molecular markers are used for identification and selection of female vs male plants in the herein disclosed breeding program
Genotyping markers—germplasm used in the current invention is genotyped using molecular markers, in order to allow a more efficient breeding process and identification of the SP and/or SP5G editing event.

It is further within the scope of the current invention that allele and genetic variation is analysed for the Cannabis strains used.

Reference is now made to optional stages that have been used for the production of mutated SP and/or SP5G Cannabis plants by genome editing:

Stage 1: Identifying Cannabis sativa (C. sativa), C. indica and C. ruderalis SP and SP5G orthologues.

Three SP orthologues have herein been identified in Cannabis sativa (C. sativa), C. indica and C. ruderalis, namely CsSP-1, CsSP-2 and CsSP-3. These homologous genes have been sequenced and mapped. CsSP-1 has been mapped to CM011605.1:71328589-71330978 and has a genomic sequence as set forth in SEQ ID NO: 1. The CsSP-1 gene has a coding sequence as set forth in SEQ ID NO: 2 and it encodes an amino acid sequence as set forth in SEQ ID NO: 3.

CsSP-2 has been mapped to CM011605.1:25325478-25326672 and has a genomic sequence as set forth in SEQ ID NO: 4. The CsSP-2 gene has a coding sequence as set forth in SEQ ID NO: 5 and it encodes an amino acid sequence as set forth in SEQ ID NO: 6.

CsSP-3 has been mapped to CM011608.1:9602945-9603900 and has a genomic sequence as set forth in SEQ ID NO: 7. The CsSP-3 gene has a coding sequence as set forth in SEQ ID NO: 8 and it encodes an amino acid sequence as set forth in SEQ ID NO: 9.

Four SP5G orthologues have herein been identified in Cannabis sativa (C. sativa), C. indica and C. ruderalis, namely CsSP5G-1, CsSP5G-2, CsSP5G-3 and CsSP5G-4. These homologous genes have been sequenced and mapped. CsSP5G-1 has been mapped to CM011610.1:5735300-5738406 and has a genomic sequence as set forth in SEQ ID NO: 10. The CsSP-1 gene has a coding sequence as set forth in SEQ ID NO: 11 and it encodes an amino acid sequence as set forth in SEQ ID NO: 12.

CsSP5G-2 has been mapped to CM011610.1:6032638-6035504 and has a genomic sequence as set forth in SEQ ID NO: 13. The CsSP5G-2 gene has a coding sequence as set forth in SEQ ID NO: 14 and it encodes an amino acid sequence as set forth in SEQ ID NO: 15.

CsSP5G-3 has been mapped to CM011607.1:79899046-79900718 and has a genomic sequence as set forth in SEQ ID NO: 16. The CsSP5G-3 gene has a coding sequence as set forth in SEQ ID NO: 17 and it encodes an amino acid sequence as set forth in SEQ ID NO: 18.

CsSP5G-4 has been mapped to CM011614.1:9255475-9256908 and has a genomic sequence as set forth in SEQ ID NO: 19. The CsSP5G-4 gene has a coding sequence as set forth in SEQ ID NO: 20 and it encodes an amino acid sequence as set forth in SEQ ID NO: 21.

Stage 2: Designing and synthesizing gRNA molecules corresponding to the sequence targeted for editing, i.e. sequences of each of the genes CsSP-1, CsSP-2 and CsSP-3; and CsSP5G-1, CsSP5G-2, CsSP5G-3 and CsSP5G-4. It is noted that the editing event is preferably targeted to a unique restriction site sequence to allow easier screening for plants carrying an editing event within their genome. According to some aspects of the invention, the nucleotide sequence of the gRNAs should be completely compatible with the genomic sequence of the target gene. Therefore, for example, suitable gRNA molecules should be constructed for different SP and/or SP5G homologues of different Cannabis strains.

Reference is now made to Tables 1-3 presenting gRNA molecules targeted for silencing CsSP-1, CsSP-2 and CsSP-3, respectively; and Tables 4-7 presenting gRNA molecules targeted for silencing CsSP5G-1, CsSP5G-2, CsSP5G-3 and CsSP5G-4 respectively. The term ‘PAM’ refers hereinafter to Protospacer Adjacent Motif, which is a 2-6 base pair DNA sequence immediately following the DNA sequence targeted by the Cas9 nuclease in the CRISPR bacterial adaptive immune system.

TABLE 1
gRNA sequences targeted for CsSP-1

| Posi- | | | | | |
|---|---|---|---|---|---|
| tion | | | | | |
| on | | | | | |
| SEQ. | | | | | SEQ. |
| ID. | | | | Efficiency | ID. |
| NO: 1 | Strand | Sequence | PAM | Score | NO. |
| 831 | 1 | TATATATATTAAGACTACGT | AGG | 69.79075 | 22 |
| 868 | −1 | ATAAGTGTGTAAGAGGCTCG | TGG | 66.70092 | 23 |
| 875 | −1 | TTATTATATAAGTGTGTAAG | AGG | 54.32121 | 24 |
| 964 | −1 | GCATGCATGCATGCATGCAT | GGG | 48.94366 | 25 |
| 965 | −1 | TGCATGCATGCATGCATGCA | TGG | 51.6943 | 26 |
| 1049 | 1 | TTGAAGAAAAGAGCAGCCAC | AGG | 56.36067 | 27 |
| 1052 | 1 | AAGAAAAGAGCAGCCACAGG | AGG | 61.86857 | 28 |
| 1054 | −1 | AAATGACCTTGGTCCTCCTG | TGG | 59.43912 | 29 |
| 1059 | 1 | GAGCAGCCACAGGAGGACCA | AGG | 64.32062 | 30 |
| 1065 | −1 | TTTGCTACCCAAAATGACCT | TGG | 60.28351 | 31 |
| 1068 | 1 | CAGGAGGACCAAGGTCATTT | TGG | 26.32254 | 32 |
| 1069 | 1 | AGGAGGACCAAGGTCATTTT | GGG | 28.83447 | 33 |
| 1080 | 1 | GGTCATTTTGGGTAGCAAAG | AGG | 69.55719 | 34 |
| 1109 | 1 | TTGAAACGCTCTCTCGATGA | AGG | 54.24251 | 35 |
| 1112 | 1 | AAACGCTCTCTCGATGAAGG | TGG | 60.45312 | 36 |
| 1129 | −1 | ACAGAAGAGAAGACAAACAG | TGG | 69.59222 | 37 |
| 1172 | −1 | GACCAAACATAGGAATACAT | AGG | 61.3781 | 38 |
| 1181 | 1 | AACCTATGTATTCCTATGTT | TGG | 36.10233 | 39 |
| 1182 | −1 | GAAATGCCACGACCAAACAT | AGG | 59.3397 | 40 |
| 1187 | 1 | TGTATTCCTATGTTTGGTCG | TGG | 47.61837 | 41 |
| 1224 | −1 | TTAAATGAATTAATATATGT | AGG | 44.76165 | 42 |
| 1324 | −1 | GGAACAACTGATGTGTCATT | TGG | 39.45477 | 43 |
| 1338 | 1 | AATGACACATCAGTTGTTCC | TGG | 39.81732 | 44 |
| 1345 | −1 | AGGATAGTGACAGACATTCC | AGG | 51.58159 | 45 |
| 1365 | −1 | TTTGTTGTTGAAATAATTGC | AGG | 31.23807 | 46 |
| 1424 | −1 | GCAAAAGGCAAAATTTAAAA | TGG | 25.80444 | 47 |
| 1439 | −1 | TTTAATCAAACTTAAGCAAA | AGG | 41.06861 | 48 |
| 1478 | 1 | TATTTTGTTAAATTATAAAT | TGG | 18.56233 | 49 |
| 1528 | 1 | TAGTATATATATATTCTGAT | TGG | 48.76865 | 50 |
| 1572 | 1 | GCAATAATAATTTAATGTAT | AGG | 42.88178 | 51 |
| 1631 | 1 | TTAAAAAATCTTCTTCAAGT | TGG | 45.32853 | 52 |
| 1755 | −1 | TTATCTAGGGTTATAATAGT | TGG | 38.51395 | 53 |
| 1768 | −1 | GTATACACATATATTATCTA | GGG | 52.79043 | 54 |
| 1769 | −1 | TGTATACACATATATTATCT | AGG | 40.10661 | 55 |
| 1881 | 1 | TCATTCAAAAGTAAAATAAT | AGG | 21.75194 | 56 |
| 1882 | 1 | CATTCAAAAGTAAAATAATA | GGG | 35.40844 | 57 |
| 1890 | 1 | AGTAAAATAATAGGGTAATT | AGG | 22.83575 | 58 |
| 1910 | −1 | TAGTAATATCAAAATTTTAA | GGG | 28.45209 | 59 |
| 1911 | −1 | TTAGTAATATCAAAATTTTA | AGG | 17.72295 | 60 |
| 1990 | 1 | TATTGAGATTGTTAAATTTA | AGG | 6.261185 | 61 |
| 2063 | −1 | TTGGTAGATATTAAAATTTA | GGG | 23.20311 | 62 |
| 2064 | −1 | TTTGGTAGATATTAAAATTT | AGG | 24.70598 | 63 |
| 2082 | −1 | GAAAGTTCAGAGGCATGATT | TGG | 33.64684 | 64 |
| 2092 | −1 | TAACATGGAGGAAAGTTCAG | AGG | 66.62639 | 65 |
| 2104 | −1 | AAAAAAACTAATTAACATGG | AGG | 67.7548 | 66 |
| 2107 | −1 | AGTAAAAAAACTAATTAACA | TGG | 49.05741 | 67 |
| 2126 | 1 | ATTAGTTTTTTTACTAAAAT | TGG | 20.24538 | 68 |
| 2154 | −1 | CTCTAAACTATTGAGATTGT | TGG | 27.96835 | 69 |
| 2214 | 1 | ATAATATAATATATATATAT | TGG | 33.84373 | 70 |
| 2365 | 1 | GACTAATTAATGATCATGTG | TGG | 67.15733 | 71 |
| 2382 | −1 | CTTAAGGGAATATATGCAAC | TGG | 42.83721 | 72 |
| 2397 | −1 | ATAATAATAAGTATACTTAA | GGG | 44.75504 | 73 |
| 2398 | −1 | TATAATAATAAGTATACTTA | AGG | 31.14549 | 74 |
| 2426 | −1 | TATATAATATATATATATAG | GGG | 51.83062 | 75 |
| 2427 | −1 | ATATATAATATATATATATA | GGG | 25.09287 | 76 |
| 2428 | −1 | TATATATAATATATATATAT | AGG | 21.06967 | 77 |
| 2527 | −1 | TGATCGATCGTTAGGGGGGA | AGG | 55.11339 | 78 |
| 2531 | −1 | ATCTTGATCGATCGTTAGGG | GGG | 69.00871 | 79 |
| 2532 | −1 | CATCTTGATCGATCGTTAGG | GGG | 62.62737 | 80 |
| 2533 | −1 | ACATCTTGATCGATCGTTAG | GGG | 62.65508 | 81 |
| 2534 | −1 | CACATCTTGATCGATCGTTA | GGG | 48.84918 | 82 |
| 2535 | −1 | ACACATCTTGATCGATCGTT | AGG | 40.46896 | 83 |
| 2692 | 1 | ATATAAGAAATATGTATGAT | CGG | 52.65835 | 84 |
| 2703 | 1 | ATGTATGATCGGAATTTTAT | TGG | 27.65155 | 85 |
| 2740 | 1 | TCAATATTATATATAGTATT | AGG | 34.80095 | 86 |
| 2818 | 1 | AACTTATATATGAATATTAT | AGG | 22.13664 | 87 |
| 2872 | −1 | TGTGATGACAAAAGTTTTTT | GGG | 25.03809 | 88 |
| 2873 | −1 | GTGTGATGACAAAAGTTTTT | TGG | 30.69267 | 89 |
| 2982 | −1 | ATTTGAGGGAGCATTTACAC | TGG | 63.0929 | 90 |
| 2996 | −1 | CCCTAGTGATCCTTATTTGA | GGG | 33.88568 | 91 |
| 2997 | 1 | TGTAAATGCTCCCTCAAATA | AGG | 33.96309 | 92 |
| 2997 | −1 | GCCCTAGTGATCCTTATTTG | AGG | 41.63481 | 93 |
| 3006 | 1 | TCCCTCAAATAAGGATCACT | AGG | 53.28568 | 94 |
| 3007 | 1 | CCCTCAAATAAGGATCACTA | GGG | 61.56296 | 95 |
| 3012 | 1 | AAATAAGGATCACTAGGGCC | AGG | 51.65425 | 96 |
| 3019 | −1 | ATGACTGATCCTGATGTTCC | TGG | 34.06368 | 97 |
| 3021 | 1 | TCACTAGGGCCAGGAACATC | AGG | 43.87242 | 98 |
| 3044 | −1 | TGTTGTAAATAATATTAATT | AGG | 20.85671 | 99 |
| 3141 | −1 | CATGAGATCCTTTTTCACAC | TGG | 54.53186 | 100 |
| 3144 | 1 | AATTATTACCAGTGTGAAAA | AGG | 40.03344 | 101 |
| 3167 | −1 | CCAAAAGTTGAGGTTCATGG | TGG | 71.52152 | 102 |
| 3170 | −1 | AAGCCAAAAGTTGAGGTTCA | TGG | 48.47928 | 103 |
| 3177 | −1 | TACTATCAAGCCAAAAGTTG | AGG | 51.74276 | 104 |
| 3178 | 1 | CCACCATGAACCTCAACTTT | TGG | 32.68026 | 105 |
| 3195 | 1 | TTTTGGCTTGATAGTAATTG | TGG | 54.13253 | 106 |
| 3199 | 1 | GGCTTGATAGTAATTGTGGA | AGG | 53.09516 | 107 |
| 3224 | −1 | TCAAACAAGAAAGTCTACAA | TGG | 60.73706 | 108 |
| 3260 | 1 | TGTAAGTCACTGTAAATTTT | AGG | 23.13386 | 109 |
| 3261 | 1 | GTAAGTCACTGTAAATTTTA | GGG | 24.03811 | 110 |
| 3265 | 1 | GTCACTGTAAATTTTAGGGT | TGG | 47.51408 | 111 |
| 3291 | −1 | TGGAAGAGTGATAGGAGATG | TGG | 64.74459 | 112 |
| 3299 | −1 | CTTATTATTGGAAGAGTGAT | AGG | 48.45991 | 113 |
| 3311 | −1 | TCAGATAATCCTCTTATTAT | TGG | 18.16136 | 114 |
| 3313 | 1 | ATCACTCTTCCAATAATAAG | AGG | 52.38055 | 115 |
| 3342 | −1 | ATTTTTTTTTAAAAAATTAA | TGG | 17.24296 | 116 |
| 3444 | −1 | TGTGTCCTCTCTATAAATAA | GGG | 40.2693 | 117 |
| 3445 | −1 | TTGTGTCCTCTCTATAAATA | AGG | 27.63962 | 118 |
| 3450 | 1 | TATATCCCTTATTTATAGAG | AGG | 51.01762 | 119 |
| 3488 | 1 | GAGATGTTTAATATATTAAG | TGG | 50.52582 | 120 |
| 3489 | 1 | AGATGTTTAATATATTAAGT | GGG | 41.77524 | 121 |
| 3490 | 1 | GATGTTTAATATATTAAGTG | GGG | 62.38698 | 122 |
| 3534 | 1 | AACTAAAATAATTTAAATGA | AGG | 46.81791 | 123 |
| 3549 | 1 | AATGAAGGAATAGTATAAAA | AGG | 26.26061 | 124 |
| 3550 | 1 | ATGAAGGAATAGTATAAAAA | GGG | 42.43128 | 125 |
| 3567 | 1 | AAAGGGTCATCATCTTCAAA | AGG | 43.17344 | 126 |

TABLE 2
gRNA sequences targeted for CsSP-2

| Posi- | | | | | |
|---|---|---|---|---|---|
| tion | | | | | |
| on | | | | | |
| SEQ. | | | | | SEQ. |
| ID. | | | | Efficiency | ID. |
| NO: 4 | Strand | Sequence | PAM | Score | NO. |
| 743 | −1 | TTTTTATCTTATCATGTTGT | TGG | 25.41093 | 127 |
| 773 | −1 | TGTAAGAGAACAGCTCTTAA | GGG | 32.52846 | 128 |
| 774 | −1 | TTGTAAGAGAACAGCTCTTA | AGG | 31.39567 | 129 |
| 812 | −1 | ACTAGTTTTAGTACGATGAT | TGG | 52.94793 | 130 |
| 907 | 1 | AATAAGAGATTAACCGTGCG | AGG | 59.61949 | 131 |
| 909 | −1 | TAGTTTTCTTTTTCCTCGCA | CGG | 48.17976 | 132 |
| 967 | 1 | TATTATATATATGTCATTAT | TGG | 18.73491 | 133 |
| 971 | 1 | ATATATATGTCATTATTGGC | AGG | 41.76418 | 134 |
| 972 | 1 | TATATATGTCATTATTGGCA | GGG | 54.92986 | 135 |
| 1048 | 1 | ATTGAAGTATACAATAGCCA | CGG | 64.48335 | 136 |
| 1049 | 1 | TTGAAGTATACAATAGCCAC | GGG | 53.86531 | 137 |
| 1050 | 1 | TGAAGTATACAATAGCCACG | GGG | 75.49947 | 138 |
| 1054 | −1 | CAACAATCTGGGTCTCCCCG | TGG | 73.79643 | 139 |
| 1065 | −1 | TTTTCTGAAGACAACAATCT | GGG | 53.83692 | 140 |
| 1066 | −1 | ATTTTCTGAAGACAACAATC | TGG | 44.86974 | 141 |
| 1091 | −1 | TGAGAGACTGTTTTAACACA | AGG | 59.78212 | 142 |
| 1109 | 1 | TTAAAACAGTCTCTCAAAGC | TGG | 53.49504 | 143 |
| 1140 | −1 | TTCATCTTATTCAAGCAAAG | AGG | 64.11009 | 144 |
| 1178 | 1 | AATCTATGAATCCCCACCAC | CGG | 66.90318 | 145 |
| 1178 | −1 | AAGCTCCAAAACCGGTGGTG | GGG | 60.93072 | 146 |
| 1179 | −1 | GAAGCTCCAAAACCGGTGGT | GGG | 54.2135 | 147 |
| 1180 | −1 | TGAAGCTCCAAAACCGGTGG | TGG | 46.33848 | 148 |
| 1183 | −1 | TTATGAAGCTCCAAAACCGG | TGG | 68.22724 | 149 |
| 1184 | 1 | TGAATCCCCACCACCGGTTT | TGG | 29.72103 | 150 |
| 1186 | −1 | GAGTTATGAAGCTCCAAAAC | CGG | 48.10068 | 151 |
| 1221 | −1 | ATATATTTATAAACGAAAAC | AGG | 40.25381 | 152 |
| 1276 | −1 | TAACGAAATAAATATCTAAA | TGG | 33.5519 | 153 |
| 1329 | −1 | GTTTTTTTCTTTTGGTAGTG | TGG | 38.70555 | 154 |
| 1337 | −1 | TGCATTATGTTTTTTTCTTT | TGG | 14.19145 | 155 |
| 1351 | 1 | AAAGAAAAAAACATAATGCA | TGG | 63.51026 | 156 |
| 1388 | 1 | CACACATATATACGATAGTG | AGG | 59.34196 | 157 |
| 1409 | 1 | GGACATGAAATACTCACCAA | AGG | 69.78964 | 158 |
| 1414 | −1 | GGTACAACTAATGTATCCTT | TGG | 46.18244 | 159 |
| 1428 | 1 | AAGGATACATTAGTTGTACC | TGG | 57.10327 | 160 |
| 1435 | −1 | AGGATAGTCACAGACATTCC | AGG | 50.29588 | 161 |
| 1448 | 1 | TGGAATGTCTGTGACTATCC | TGG | 45.01183 | 162 |
| 1455 | −1 | ATAATACGTGTATTCTTTCC | AGG | 31.45682 | 163 |
| 1488 | −1 | GTGATCCTTTCCATTAATAT | TGG | 33.16337 | 164 |
| 1489 | 1 | AATTAAATATCCAATATTAA | TGG | 20.04105 | 165 |
| 1494 | 1 | AATATCCAATATTAATGGAA | AGG | 55.69722 | 166 |
| 1532 | 1 | ATATATAAATATACAAGAAG | AGG | 52.03505 | 167 |
| 1606 | 1 | ATTATATTACAAACCAGTGA | AGG | 62.03101 | 168 |
| 1608 | −1 | ACTTAAAAGAACACCTTCAC | TGG | 43.10988 | 169 |
| 1622 | 1 | GTGAAGGTGTTCTTTTAAGT | AGG | 40.61279 | 170 |
| 1623 | 1 | TGAAGGTGTTCTTTTAAGTA | GGG | 44.06002 | 171 |
| 1632 | 1 | TCTTTTAAGTAGGGATCACT | AGG | 52.1437 | 172 |
| 1633 | 1 | CTTTTAAGTAGGGATCACTA | GGG | 60.66675 | 173 |
| 1638 | 1 | AAGTAGGGATCACTAGGGCT | TGG | 43.63704 | 174 |
| 1647 | 1 | TCACTAGGGCTTGGAGCATC | TGG | 33.29868 | 175 |
| 1664 | −1 | CACTTATTTCTTGAAGATTA | TGG | 30.34701 | 176 |
| 1676 | 1 | CATAATCTTCAAGAAATAAG | TGG | 51.71581 | 177 |
| 1684 | 1 | TCAAGAAATAAGTGGAAAAA | AGG | 33.99747 | 178 |
| 1685 | 1 | CAAGAAATAAGTGGAAAAAA | GGG | 41.53648 | 179 |
| 1815 | −1 | AAACCTCGAGTAGATATTGG | TGG | 58.65631 | 180 |
| 1818 | −1 | TCTAAACCTCGAGTAGATAT | TGG | 41.98098 | 181 |
| 1823 | 1 | TCTCCACCAATATCTACTCG | AGG | 63.24041 | 182 |
| 1844 | 1 | GGTTTAGACAAAATATTAGA | AGG | 43.87082 | 183 |
| 1869 | −1 | CCTGATAAGCAAGTTTGTAA | TGG | 46.15333 | 184 |
| 1880 | 1 | CCATTACAAACTTGCTTATC | AGG | 22.76102 | 185 |
| 1881 | 1 | CATTACAAACTTGCTTATCA | GGG | 46.00555 | 186 |
| 1913 | 1 | ATGACAGTCATTTTTACACT | AGG | 56.38646 | 187 |
| 1939 | −1 | GGCAAGAGTAATAGGAGATG | TGG | 67.01531 | 188 |
| 1947 | −1 | TTAAGTGTGGCAAGAGTAAT | AGG | 35.65879 | 189 |
| 1960 | −1 | AGCAATACAAACTTTAAGTG | TGG | 64.17131 | 190 |
| 1985 | −1 | TTTATATAATAATAATGTCA | AGG | 61.85855 | 191 |
| 2025 | 1 | AATAGATCAGATATATGATA | AGG | 43.78777 | 192 |
| 2033 | 1 | AGATATATGATAAGGATGAG | AGG | 64.39285 | 193 |
| 2104 | 1 | GTAGTAGTAGAGATCGAGTT | AGG | 52.76822 | 194 |
| 2114 | 1 | AGATCGAGTTAGGTGATCAA | TGG | 47.8583 | 195 |
| 2115 | 1 | GATCGAGTTAGGTGATCAAT | GGG | 43.40038 | 196 |
| 2134 | 1 | TGGGTATTTATAAGCCAGCT | AGG | 53.71914 | 197 |
| 2135 | 1 | GGGTATTTATAAGCCAGCTA | GGG | 59.56206 | 198 |
| 2137 | −1 | AAATTCCAACTTGCCCTAGC | TGG | 36.84358 | 199 |
| 2143 | 1 | ATAAGCCAGCTAGGGCAAGT | TGG | 60.49436 | 200 |
| 2187 | 1 | TTATTTGAATAGCGTAGTAG | TGG | 54.61689 | 201 |
| 2197 | 1 | AGCGTAGTAGTGGCTGCTCT | TGG | 43.97502 | 202 |
| 2198 | 1 | GCGTAGTAGTGGCTGCTCTT | GGG | 44.41787 | 203 |
| 2208 | 1 | GGCTGCTCTTGGGAAATGCC | AGG | 52.38622 | 204 |
| 2215 | −1 | AATAAAGCAAATATTAAACC | TGG | 60.29947 | 205 |
| 2253 | 1 | GTAAAAAGTTAAAATACCAA | AGG | 65.71087 | 206 |
| 2258 | −1 | GTGATGTTTTCAAATTCCTT | TGG | 44.14554 | 207 |
| 2309 | −1 | CATACGTGTCGAACTAGGAA | TGG | 55.15684 | 208 |
| 2314 | −1 | ACAACCATACGTGTCGAACT | AGG | 63.97993 | 209 |
| 2321 | 1 | CATTCCTAGTTCGACACGTA | TGG | 57.55443 | 210 |
| 2372 | 1 | TTGTTGATGTTGAAAGAAGT | TGG | 64.04881 | 211 |

TABLE 3
gRNA sequences targeted for CsSP-3

| Posi- | | | | | |
|---|---|---|---|---|---|
| tion | | | | | |
| on | | | | | |
| SEQ. | | | | | SEQ. |
| ID. | | | | Efficiency | ID. |
| NO: 7 | Strand | Sequence | PAM | Score | NO. |
| 979 | 1 | AGAATATATATATATATTAG | TGG | 52.75818 | 212 |
| 983 | 1 | TATATATATATATTAGTGGT | AGG | 56.18088 | 213 |
| 987 | 1 | TATATATATTAGTGGTAGGT | AGG | 46.88074 | 214 |
| 1012 | 1 | GAGCTAGCGTCTTCTAGCTG | CGG | 57.33903 | 215 |
| 1039 | 1 | TCTCTGAGCGTTGAAATAAA | CGG | 31.00695 | 216 |
| 1049 | 1 | TTGAAATAAACGGCAGCGAC | AGG | 51.04418 | 217 |
| 1059 | 1 | CGGCAGCGACAGGAAGACCG | AGG | 71.16066 | 218 |
| 1065 | −1 | TTCGCTGCTCAAAACGACCT | CGG | 66.77634 | 219 |
| 1108 | −1 | GGTGAGTATACCAAGCTCAA | AGG | 64.31989 | 220 |
| 1109 | 1 | TTGAAATGATCCTTTGAGCT | TGG | 55.54256 | 221 |
| 1128 | 1 | TTGGTATACTCACCGAGTCT | CGG | 54.52127 | 222 |
| 1129 | −1 | CAAACAGAAATGCCGAGACT | CGG | 54.73946 | 223 |
| 1169 | −1 | AGCCAAATATAGGGATTCAC | AGG | 49.91997 | 224 |
| 1178 | 1 | AACCTGTGAATCCCTATATT | TGG | 23.1867 | 225 |
| 1178 | −1 | AGAGCCCAAAGCCAAATATA | GGG | 42.41948 | 226 |
| 1179 | −1 | GAGAGCCCAAAGCCAAATAT | AGG | 31.63154 | 227 |
| 1184 | 1 | TGAATCCCTATATTTGGCTT | TGG | 26.63952 | 228 |
| 1185 | 1 | GAATCCCTATATTTGGCTTT | GGG | 29.53778 | 229 |
| 1210 | −1 | TATGTACACAGGAAGGGAAG | TGG | 62.37694 | 230 |
| 1216 | −1 | AATGGATATGTACACAGGAA | GGG | 56.73565 | 231 |
| 1217 | −1 | AAATGGATATGTACACAGGA | AGG | 56.69376 | 232 |
| 1221 | −1 | ATTAAAATGGATATGTACAC | AGG | 64.92133 | 233 |
| 1234 | −1 | TTATAAATATAAAATTAAAA | TGG | 25.89586 | 234 |
| 1279 | 1 | TATACATATATAATTAATAA | TGG | 34.02683 | 235 |
| 1309 | −1 | GGCACCACAGACGCTACATT | TGG | 42.45215 | 236 |
| 1316 | 1 | ATTACCAAATGTAGCGTCTG | TGG | 57.76274 | 237 |
| 1323 | 1 | AATGTAGCGTCTGTGGTGCC | TGG | 52.49826 | 238 |
| 1330 | −1 | AGGATTGTGACAGACATACC | AGG | 64.90611 | 239 |
| 1350 | −1 | TTCTTCTTTGAAACATGGAC | AGG | 56.04975 | 240 |
| 1355 | −1 | TTATTTTCTTCTTTGAAACA | TGG | 51.31281 | 241 |
| 1380 | 1 | AGAAAATAATTATTACAATC | AGG | 48.41061 | 242 |
| 1390 | 1 | TATTACAATCAGGATATCTT | AGG | 46.56003 | 243 |
| 1441 | −1 | TTTAATTAACAAACTATTTT | AGG | 18.17429 | 244 |
| 1464 | −1 | ATGTCAGTAAGATTATAATT | TGG | 33.42864 | 245 |
| 1479 | 1 | ATTATAATCTTACTGACATT | TGG | 32.26823 | 246 |
| 1522 | 1 | ACTATTTATTATACCAGTGA | AGG | 55.21337 | 247 |
| 1524 | −1 | ACTTAAGGGAGCACCTTCAC | TGG | 49.73888 | 248 |
| 1538 | −1 | CCCAAGTGATCCTTACTTAA | GGG | 38.09188 | 249 |
| 1539 | 1 | TGAAGGTGCTCCCTTAAGTA | AGG | 48.60231 | 250 |
| 1539 | −1 | GCCCAAGTGATCCTTACTTA | AGG | 40.75021 | 251 |
| 1548 | 1 | TCCCTTAAGTAAGGATCACT | TGG | 45.83122 | 252 |
| 1549 | 1 | CCCTTAAGTAAGGATCACTT | GGG | 69.93595 | 253 |
| 1554 | 1 | AAGTAAGGATCACTTGGGCC | AGG | 52.25638 | 254 |
| 1561 | −1 | ATGACAGATCCAGATGTTCC | TGG | 43.93102 | 255 |
| 1563 | 1 | TCACTTGGGCCAGGAACATC | TGG | 40.0198 | 256 |
| 1586 | −1 | CTCTCTCTTTATTTTATTTT | AGG | 4.865075 | 257 |
| 1623 | −1 | AATTAATGTTGTTCTTTTTT | TGG | 15.45049 | 258 |
| 1663 | −1 | AGATCTCTAAATGATGAACA | AGG | 65.19671 | 259 |
| 1722 | 1 | TTTTTTTATTAGTGTGAAGA | AGG | 48.17114 | 260 |
| 1748 | −1 | AAACCTAGGGTTGAGATTCA | AGG | 38.00239 | 261 |
| 1756 | 1 | GCTCCTTGAATCTCAACCCT | AGG | 58.80331 | 262 |
| 1761 | 1 | TTGAATCTCAACCCTAGGTT | TGG | 37.75685 | 263 |
| 1761 | −1 | AGTTGTCATCTCCAAACCTA | GGG | 52.43725 | 264 |
| 1762 | −1 | CAGTTGTCATCTCCAAACCT | AGG | 57.30496 | 265 |
| 1777 | 1 | GGTTTGGAGATGACAACTGA | AGG | 63.04415 | 266 |
| 1802 | −1 | AACAAGAAGCAAGTCTGTAA | TGG | 45.44633 | 267 |
| 1836 | 1 | ATAAGAGACAGACATTTTTA | TGG | 28.08049 | 268 |
| 1840 | 1 | GAGACAGACATTTTTATGGA | TGG | 52.94092 | 269 |
| 1874 | −1 | CTTGTGGTTGGAAGAGTGAT | AGG | 54.20481 | 270 |
| 1886 | −1 | ATGTTAGAGCCTCTTGTGGT | TGG | 53.01781 | 271 |
| 1888 | 1 | ATCACTCTTCCAACCACAAG | AGG | 57.38136 | 272 |
| 1890 | −1 | AAGAATGTTAGAGCCTCTTG | TGG | 65.83401 | 273 |
| 1914 | −1 | TATTACTATAAATATAATTA | TGG | 28.32586 | 274 |
| 1950 | −1 | ATATATTTTAAGTACTTAAT | TGG | 18.0389 | 275 |
| 2019 | 1 | TTTATATATGTGTTTGTGTG | TGG | 49.7003 | 276 |
| 2060 | 1 | AATTATTACTTAGTTCGTGA | TGG | 53.17869 | 277 |
| 2065 | 1 | TTACTTAGTTCGTGATGGAA | TGG | 52.02412 | 278 |
| 2072 | 1 | GTTCGTGATGGAATGGAACT | TGG | 45.80324 | 279 |
| 2087 | −1 | TGTAGACACTATAAATACAT | GGG | 59.04909 | 280 |
| 2088 | −1 | ATGTAGACACTATAAATACA | TGG | 56.82992 | 281 |
| 2110 | 1 | TAGTGTCTACATGATATCTT | TGG | 36.6185 | 282 |
| 2124 | −1 | TAAAAGTAAAGTTACAATTA | AGG | 32.62771 | 283 |

TABLE 4
gRNA sequences targeted for CsSP5G-1

| Posi- | | | | | |
|---|---|---|---|---|---|
| tion | | | | | |
| on | | | | | |
| SEQ. | | | | | SEQ. |
| ID. | | | | Efficiency | ID. |
| NO: 10 | Strand | Sequence | PAM | Score | NO. |
| 43 | 1 | ATATTAATATATAACAAGTT | TGG | 33.4577 | 284 |
| 127 | 1 | AATAATTAATTAAGAATATA | TGG | 32.43589 | 285 |
| 138 | 1 | AAGAATATATGGCTAGAGAT | AGG | 45.23675 | 286 |
| 139 | 1 | AGAATATATGGCTAGAGATA | GGG | 53.98613 | 287 |
| 151 | 1 | TAGAGATAGGGACCCTCTTG | TGG | 52.53234 | 288 |
| 152 | −1 | TTACTCTACCAACCACAAGA | GGG | 67.79474 | 289 |
| 153 | −1 | ATTACTCTACCAACCACAAG | AGG | 59.18615 | 290 |
| 155 | 1 | GATAGGGACCCTCTTGTGGT | TGG | 56.67036 | 291 |
| 167 | 1 | CTTGTGGTTGGTAGAGTAAT | AGG | 35.27607 | 292 |
| 178 | 1 | TAGAGTAATAGGAGATGTTT | TGG | 32.95839 | 293 |
| 192 | 1 | ATGTTTTGGATCCTTTTACA | AGG | 55.39173 | 294 |
| 192 | −1 | AGAGAGACTGACCTTGTAAA | AGG | 42.08228 | 295 |
| 218 | 1 | GTCTCTCTTAGAGTGAGTTA | TGG | 47.06405 | 296 |
| 229 | 1 | AGTGAGTTATGGTAATAGAG | AGG | 54.93598 | 297 |
| 239 | 1 | GGTAATAGAGAGGTCAACAA | TGG | 58.93327 | 298 |
| 264 | −1 | GGTTGGTTAACAATTTGGGA | AGG | 61.46045 | 299 |
| 268 | −1 | ACGAGGTTGGTTAACAATTT | GGG | 32.45336 | 300 |
| 269 | −1 | CACGAGGTTGGTTAACAATT | TGG | 23.81735 | 301 |
| 281 | −1 | CACCAATATCAACACGAGGT | TGG | 61.80226 | 302 |
| 285 | −1 | TCACCACCAATATCAACACG | AGG | 75.84355 | 303 |
| 290 | 1 | AACCAACCTCGTGTTGATAT | TGG | 44.46178 | 304 |
| 293 | 1 | CAACCTCGTGTTGATATTGG | TGG | 58.81318 | 305 |
| 306 | 1 | ATATTGGTGGTGATGACCTA | AGG | 60.13671 | 306 |
| 311 | −1 | CCAAAGTGTAGAAGGTCCTT | AGG | 42.09425 | 307 |
| 319 | −1 | TTAATTTACCAAAGTGTAGA | AGG | 48.09029 | 308 |
| 322 | 1 | CCTAAGGACCTTCTACACTT | TGG | 50.47553 | 309 |
| 357 | −1 | TGAAATCATTATGAATATTG | AGG | 50.25272 | 310 |
| 436 | 1 | CATATATTGAAAATTATTAC | AGG | 36.58192 | 311 |
| 442 | 1 | TTGAAAATTATTACAGGTCA | TGG | 43.08159 | 312 |
| 445 | 1 | AAAATTATTACAGGTCATGG | TGG | 65.82262 | 313 |
| 451 | 1 | ATTACAGGTCATGGTGGATC | CGG | 50.20677 | 314 |
| 459 | −1 | TTGCTAGGGCTAGGAGCATC | CGG | 49.91113 | 315 |
| 468 | −1 | AGATTGGGGTTGCTAGGGCT | AGG | 44.68519 | 316 |
| 473 | −1 | CCCTTAGATTGGGGTTGCTA | GGG | 47.67473 | 317 |
| 474 | −1 | TCCCTTAGATTGGGGTTGCT | AGG | 44.15699 | 318 |
| 482 | −1 | GCAAATACTCCCTTAGATTG | GGG | 58.46921 | 319 |
| 483 | 1 | GCCCTAGCAACCCCAATCTA | AGG | 36.0562 | 320 |
| 483 | −1 | TGCAAATACTCCCTTAGATT | GGG | 30.97985 | 321 |
| 484 | 1 | CCCTAGCAACCCCAATCTAA | GGG | 45.48361 | 322 |
| 484 | −1 | ATGCAAATACTCCCTTAGAT | TGG | 38.79211 | 323 |
| 498 | 1 | ATCTAAGGGAGTATTTGCAT | TGG | 59.37908 | 324 |
| 556 | 1 | ATATTATTATTAAATAGATG | AGG | 50.44408 | 325 |
| 557 | 1 | TATTATTATTAAATAGATGA | GGG | 52.51763 | 326 |
| 689 | 1 | TTTAATTTTGTATAAAACTT | TGG | 34.74999 | 327 |
| 808 | −1 | TTCATGCACACAACACATGT | TGG | 55.60825 | 328 |
| 869 | −1 | CAAAAAGTAAAGACATATTT | TGG | 14.91886 | 329 |
| 881 | 1 | CAAAATATGTCTTTACTTTT | TGG | 10.5253 | 330 |
| 916 | 1 | CATTTTATAAAGATGTTAGT | TGG | 33.17467 | 331 |
| 917 | 1 | ATTTTATAAAGATGTTAGTT | GGG | 40.20867 | 332 |
| 1105 | 1 | TTTTAGTGTCAGTTTTGAAT | TGG | 29.61892 | 333 |
| 1123 | −1 | ACAAAATTCTGTAATTATTA | GGG | 26.7988 | 334 |
| 1124 | −1 | TACAAAATTCTGTAATTATT | AGG | 13.97206 | 335 |
| 1156 | −1 | ACAAATTAAAACAAGCTTTA | GGG | 23.11439 | 336 |
| 1157 | −1 | AACAAATTAAAACAAGCTTT | AGG | 32.2923 | 337 |
| 1180 | 1 | TTTAATTTGTTAAAGTGACT | AGG | 54.58344 | 338 |
| 1228 | −1 | AACATGTAAAAAGAATTTAA | GGG | 33.91047 | 339 |
| 1229 | −1 | AAACATGTAAAAAGAATTTA | AGG | 15.92615 | 340 |
| 1258 | −1 | GCTAGCATATATGGAATTTG | TGG | 45.56144 | 341 |
| 1267 | −1 | ATTTATATAGCTAGCATATA | TGG | 26.77656 | 342 |
| 1288 | 1 | TAGCTATATAAATATAAATA | TGG | 35.14496 | 343 |
| 1293 | 1 | ATATAAATATAAATATGGAA | AGG | 60.53312 | 344 |
| 1301 | 1 | ATAAATATGGAAAGGATATA | TGG | 32.49959 | 345 |
| 1302 | 1 | TAAATATGGAAAGGATATAT | GGG | 33.709 | 346 |
| 1351 | 1 | AAAGCTGATGAGAAAGAATG | TGG | 66.11521 | 347 |
| 1356 | 1 | TGATGAGAAAGAATGTGGTT | TGG | 50.94728 | 348 |
| 1357 | 1 | GATGAGAAAGAATGTGGTTT | GGG | 37.02061 | 349 |
| 1358 | 1 | ATGAGAAAGAATGTGGTTTG | GGG | 59.03998 | 350 |
| 1379 | 1 | GGATGAATTTTGAATGATGA | AGG | 48.10583 | 351 |
| 1380 | 1 | GATGAATTTTGAATGATGAA | GGG | 53.0293 | 352 |
| 1386 | 1 | TTTTGAATGATGAAGGGATG | AGG | 52.46871 | 353 |
| 1398 | 1 | AAGGGATGAGGCTGTGTGTG | TGG | 55.94352 | 354 |
| 1421 | −1 | GGGACATGCTATAGCTAGCA | GGG | 60.76795 | 355 |
| 1422 | −1 | GGGGACATGCTATAGCTAGC | AGG | 49.84652 | 356 |
| 1441 | −1 | TTTTAATGGTGGGACAAAAG | GGG | 54.93961 | 357 |
| 1442 | −1 | ATTTTAATGGTGGGACAAAA | GGG | 35.09294 | 358 |
| 1443 | −1 | CATTTTAATGGTGGGACAAA | AGG | 27.24033 | 359 |
| 1451 | −1 | GAGGTGGCCATTTTAATGGT | GGG | 62.03513 | 360 |
| 1452 | −1 | TGAGGTGGCCATTTTAATGG | TGG | 64.3868 | 361 |
| 1455 | 1 | CTTTTGTCCCACCATTAAAA | TGG | 29.86891 | 362 |
| 1455 | −1 | GTGTGAGGTGGCCATTTTAA | TGG | 29.73425 | 363 |
| 1467 | −1 | AAAACCTTCTTAGTGTGAGG | TGG | 69.54075 | 364 |
| 1470 | −1 | GTGAAAACCTTCTTAGTGTG | AGG | 60.43941 | 365 |
| 1474 | 1 | ATGGCCACCTCACACTAAGA | AGG | 56.86845 | 366 |
| 1543 | 1 | ATATATACATACACATGTAT | AGG | 45.18352 | 367 |
| 1544 | 1 | TATATACATACACATGTATA | GGG | 55.90543 | 368 |
| 1612 | −1 | CTATGTTCGAATTCAAATTC | GGG | 34.62224 | 369 |
| 1613 | −1 | TCTATGTTCGAATTCAAATT | CGG | 36.42467 | 370 |
| 1635 | 1 | TTCGAACATAGACTCAGATT | TGG | 35.59295 | 371 |
| 1648 | −1 | AGGGTTCAGGGTCGAATTTA | GGG | 28.94531 | 372 |
| 1649 | −1 | CAGGGTTCAGGGTCGAATTT | AGG | 26.64298 | 373 |
| 1660 | −1 | AGTTCGTGTTTCAGGGTTCA | GGG | 43.58548 | 374 |
| 1661 | −1 | AAGTTCGTGTTTCAGGGTTC | AGG | 26.7905 | 375 |
| 1667 | −1 | GAGTCTAAGTTCGTGTTTCA | GGG | 35.03576 | 376 |
| 1668 | −1 | TGAGTCTAAGTTCGTGTTTC | AGG | 15.64056 | 377 |
| 1686 | 1 | CACGAACTTAGACTCAGACC | TGG | 55.22454 | 378 |
| 1692 | 1 | CTTAGACTCAGACCTGGACC | TGG | 54.41072 | 379 |
| 1693 | −1 | TTGGGTCAAGGTCCAGGTCC | AGG | 45.45826 | 380 |
| 1699 | −1 | CGGGGTTTGGGTCAAGGTCC | AGG | 43.16961 | 381 |
| 1705 | −1 | TCGGGTCGGGGTTTGGGTCA | AGG | 38.7222 | 382 |
| 1711 | −1 | CGGGGTTCGGGTCGGGGTTT | GGG | 29.12979 | 383 |
| 1712 | −1 | TCGGGGTTCGGGTCGGGGTT | TGG | 16.04517 | 384 |
| 1717 | −1 | TCGAGTCGGGGTTCGGGTCG | GGG | 28.40008 | 385 |
| 1718 | −1 | TTCGAGTCGGGGTTCGGGTC | GGG | 28.3022 | 386 |
| 1719 | −1 | GTTCGAGTCGGGGTTCGGGT | CGG | 44.74237 | 387 |
| 1723 | −1 | CGGGGTTCGAGTCGGGGTTC | GGG | 33.72492 | 388 |
| 1724 | −1 | TCGGGGTTCGAGTCGGGGTT | CGG | 29.847 | 389 |
| 1729 | −1 | TAGTTTCGGGGTTCGAGTCG | GGG | 54.61581 | 390 |
| 1730 | −1 | CTAGTTTCGGGGTTCGAGTC | GGG | 35.21533 | 391 |
| 1731 | −1 | TCTAGTTTCGGGGTTCGAGT | CGG | 47.1652 | 392 |
| 1741 | −1 | CCAGGTCCAGTCTAGTTTCG | GGG | 59.72421 | 393 |
| 1742 | −1 | TCCAGGTCCAGTCTAGTTTC | GGG | 29.76351 | 394 |
| 1743 | −1 | GTCCAGGTCCAGTCTAGTTT | CGG | 32.43822 | 395 |
| 1746 | 1 | CTCGAACCCCGAAACTAGAC | TGG | 41.77216 | 396 |
| 1752 | 1 | CCCCGAAACTAGACTGGACC | TGG | 44.07102 | 397 |
| 1759 | 1 | ACTAGACTGGACCTGGACTC | TGG | 56.19546 | 398 |
| 1759 | −1 | TAGGTCCTAGGCCAGAGTCC | AGG | 53.14281 | 399 |
| 1765 | 1 | CTGGACCTGGACTCTGGCCT | AGG | 55.36929 | 400 |
| 1771 | −1 | CTAGACCCGAGCTAGGTCCT | AGG | 55.5272 | 401 |
| 1776 | 1 | CTCTGGCCTAGGACCTAGCT | CGG | 44.89925 | 402 |
| 1777 | 1 | TCTGGCCTAGGACCTAGCTC | GGG | 55.94427 | 403 |
| 1778 | −1 | CTGAACTCTAGACCCGAGCT | AGG | 64.71982 | 404 |
| 1790 | 1 | CTAGCTCGGGTCTAGAGTTC | AGG | 38.28516 | 405 |
| 1800 | 1 | TCTAGAGTTCAGGTCCAGTC | CGG | 44.68981 | 406 |
| 1801 | 1 | CTAGAGTTCAGGTCCAGTCC | GGG | 51.19192 | 407 |
| 1802 | 1 | TAGAGTTCAGGTCCAGTCCG | GGG | 64.76165 | 408 |
| 1803 | −1 | CCTGACCTCGGACCCCGGAC | TGG | 42.34928 | 409 |
| 1808 | −1 | CAGATCCTGACCTCGGACCC | CGG | 54.44243 | 410 |
| 1809 | 1 | CAGGTCCAGTCCGGGGTCCG | AGG | 55.03576 | 411 |
| 1814 | 1 | CCAGTCCGGGGTCCGAGGTC | AGG | 39.80181 | 412 |
| 1815 | −1 | ACGAACCCAGATCCTGACCT | CGG | 70.13695 | 413 |
| 1820 | 1 | CGGGGTCCGAGGTCAGGATC | TGG | 27.07 | 414 |
| 1821 | 1 | GGGGTCCGAGGTCAGGATCT | GGG | 45.99404 | 415 |
| 1833 | 1 | CAGGATCTGGGTTCGTGTTC | TGG | 13.13476 | 416 |
| 1834 | 1 | AGGATCTGGGTTCGTGTTCT | GGG | 32.34104 | 417 |
| 1835 | 1 | GGATCTGGGTTCGTGTTCTG | GGG | 61.73636 | 418 |
| 1841 | 1 | GGGTTCGTGTTCTGGGGTTC | AGG | 32.84982 | 419 |
| 1845 | 1 | TCGTGTTCTGGGGTTCAGGT | TGG | 32.94333 | 420 |
| 1846 | 1 | CGTGTTCTGGGGTTCAGGTT | GGG | 37.64639 | 421 |
| 1850 | 1 | TTCTGGGGTTCAGGTTGGGT | TGG | 36.37666 | 422 |
| 1851 | 1 | TCTGGGGTTCAGGTTGGGTT | GGG | 39.38005 | 423 |
| 1856 | 1 | GGTTCAGGTTGGGTTGGGTC | TGG | 33.59531 | 424 |
| 1863 | 1 | GTTGGGTTGGGTCTGGAGTC | TGG | 19.45666 | 425 |
| 1864 | 1 | TTGGGTTGGGTCTGGAGTCT | GGG | 39.24034 | 426 |
| 1870 | 1 | TGGGTCTGGAGTCTGGGTCT | AGG | 26.29809 | 427 |
| 1871 | 1 | GGGTCTGGAGTCTGGGTCTA | GGG | 46.06491 | 428 |
| 1883 | 1 | TGGGTCTAGGGTCCAGATTC | AGG | 36.11531 | 429 |
| 1884 | −1 | CCTGAACCCGATCCTGAATC | TGG | 41.73702 | 430 |
| 1888 | 1 | CTAGGGTCCAGATTCAGGAT | CGG | 53.25207 | 431 |
| 1889 | 1 | TAGGGTCCAGATTCAGGATC | GGG | 49.77329 | 432 |
| 1895 | 1 | CCAGATTCAGGATCGGGTTC | AGG | 32.80081 | 433 |
| 1901 | 1 | TCAGGATCGGGTTCAGGTTA | AGG | 33.91225 | 434 |
| 1919 | 1 | TAAGGTTTGAGTCTGAGTCC | AGG | 59.59865 | 435 |
| 1925 | 1 | TTGAGTCTGAGTCCAGGTAT | AGG | 52.86993 | 436 |
| 1926 | −1 | TCCCGACCAGAACCTATACC | TGG | 43.98773 | 437 |
| 1931 | 1 | CTGAGTCCAGGTATAGGTTC | TGG | 45.95561 | 438 |
| 1935 | 1 | GTCCAGGTATAGGTTCTGGT | CGG | 54.99259 | 439 |
| 1936 | 1 | TCCAGGTATAGGTTCTGGTC | GGG | 55.16665 | 440 |
| 1964 | 1 | AGTTCGAGAGTTTGAATTCA | AGG | 34.8731 | 441 |
| 1974 | 1 | TTTGAATTCAAGGTCCAATT | TGG | 30.63103 | 442 |
| 1977 | −1 | GAACTCATCCAACTCCAAAT | TGG | 38.88806 | 443 |
| 1980 | 1 | TTCAAGGTCCAATTTGGAGT | TGG | 43.86757 | 444 |
| 1997 | 1 | AGTTGGATGAGTTCATGTCA | TGG | 67.34083 | 445 |
| 2063 | −1 | TTTAAAATTTTAATAGTGTT | TGG | 34.83327 | 446 |
| 2179 | 1 | ATTCATAATTTTTAAATTAG | AGG | 42.99532 | 447 |
| 2180 | 1 | TTCATAATTTTTAAATTAGA | GGG | 36.49527 | 448 |
| 2196 | −1 | TTATTTTTATCTTACTTATA | GGG | 25.50447 | 449 |
| 2197 | −1 | ATTATTTTTATCTTACTTAT | AGG | 20.4776 | 450 |
| 2308 | −1 | TTTACTGTACCGAATATTCA | CGG | 41.6042 | 451 |
| 2310 | 1 | TTGTAGTTACCGTGAATATT | CGG | 32.91515 | 452 |
| 2325 | 1 | ATATTCGGTACAGTAAATTA | AGG | 39.9063 | 453 |
| 2329 | 1 | TCGGTACAGTAAATTAAGGA | TGG | 57.49355 | 454 |
| 2410 | −1 | ATATATAAAAATATAAATTG | TGG | 59.46361 | 455 |
| 2441 | 1 | TATATTATTAATCTAGATAA | TGG | 49.32964 | 456 |
| 2493 | −1 | ATAATTATACTATATATTAT | AGG | 26.08693 | 457 |
| 2523 | 1 | TTATAATAATTATACATGTT | TGG | 35.08265 | 458 |
| 2538 | 1 | ATGTTTGGCAATTTCAATTT | AGG | 24.64794 | 459 |
| 2542 | 1 | TTGGCAATTTCAATTTAGGT | TGG | 51.99459 | 460 |
| 2558 | 1 | AGGTTGGTGACTGATATTCC | TGG | 39.09227 | 461 |
| 2565 | −1 | AAGCTTGGCCCGGTAGTTCC | AGG | 41.23058 | 462 |
| 2567 | 1 | ACTGATATTCCTGGAACTAC | CGG | 48.93325 | 463 |
| 2568 | 1 | CTGATATTCCTGGAACTACC | GGG | 62.10752 | 464 |
| 2575 | −1 | CGGCTCACCGAAGCTTGGCC | CGG | 48.9322 | 465 |
| 2579 | 1 | GGAACTACCGGGCCAAGCTT | CGG | 46.22153 | 466 |
| 2580 | −1 | ATGAACGGCTCACCGAAGCT | TGG | 59.46928 | 467 |
| 2595 | −1 | GTATTATTATGAAGTATGAA | CGG | 57.67305 | 468 |
| 2666 | 1 | ACGCTGTAAACAAAATAGTG | CGG | 67.72415 | 469 |
| 2768 | 1 | ATTAATTGTTTATTATGTGT | AGG | 49.28958 | 470 |
| 2776 | 1 | TTTATTATGTGTAGGACAAG | AGG | 55.5504 | 471 |
| 2779 | 1 | ATTATGTGTAGGACAAGAGG | TGG | 65.45521 | 472 |
| 2799 | 1 | TGGTGTGCTACGAGAACCCG | CGG | 69.35789 | 473 |
| 2804 | −1 | GAATCCCCACCGTCGGCCGC | GGG | 51.25283 | 474 |
| 2805 | −1 | TGAATCCCCACCGTCGGCCG | CGG | 46.05954 | 475 |
| 2806 | 1 | CTACGAGAACCCGCGGCCGA | CGG | 55.45967 | 476 |
| 2809 | 1 | CGAGAACCCGCGGCCGACGG | TGG | 52.81179 | 477 |
| 2810 | 1 | GAGAACCCGCGGCCGACGGT | GGG | 56.14245 | 478 |
| 2811 | 1 | AGAACCCGCGGCCGACGGTG | GGG | 60.30596 | 479 |
| 2811 | −1 | TACCGATGAATCCCCACCGT | CGG | 68.50256 | 480 |
| 2820 | 1 | GGCCGACGGTGGGGATTCAT | CGG | 48.29492 | 481 |
| 2841 | 1 | GGTATGTATTTGTGTTGTTC | CGG | 42.62051 | 482 |
| 2848 | 1 | ATTTGTGTTGTTCCGGCAAT | TGG | 33.46831 | 483 |
| 2849 | 1 | TTTGTGTTGTTCCGGCAATT | GGG | 46.97766 | 484 |
| 2849 | −1 | CCGTTTGCCTTCCCAATTGC | CGG | 39.46262 | 485 |
| 2853 | 1 | TGTTGTTCCGGCAATTGGGA | AGG | 55.97024 | 486 |
| 2860 | 1 | CCGGCAATTGGGAAGGCAAA | CGG | 54.12282 | 487 |
| 2872 | 1 | AAGGCAAACGGTGTTCGCGC | CGG | 41.65064 | 488 |
| 2873 | 1 | AGGCAAACGGTGTTCGCGCC | GGG | 37.60685 | 489 |
| 2874 | 1 | GGCAAACGGTGTTCGCGCCG | GGG | 56.96599 | 490 |
| 2877 | 1 | AAACGGTGTTCGCGCCGGGG | TGG | 55.4399 | 491 |
| 2880 | −1 | TTGAAGTTCTGACGCCACCC | CGG | 59.80253 | 492 |
| 2905 | −1 | ATAAAGCTCAGCAAAGTCTT | TGG | 43.946 | 493 |
| 2924 | 1 | TTTGCTGAGCTTTATAACCT | TGG | 44.20648 | 494 |
| 2930 | −1 | GGGCAGCAACAGGCAAACCA | AGG | 68.3844 | 495 |
| 2940 | −1 | TTGTAATAAAGGGCAGCAAC | AGG | 46.09672 | 496 |
| 2950 | −1 | CCTTTGGCAGTTGTAATAAA | GGG | 32.56095 | 497 |
| 2951 | −1 | CCCTTTGGCAGTTGTAATAA | AGG | 29.14878 | 498 |
| 2961 | 1 | CCCTTTATTACAACTGCCAA | AGG | 54.00419 | 499 |
| 2962 | 1 | CCTTTATTACAACTGCCAAA | GGG | 58.42829 | 500 |
| 2966 | −1 | CCCCAGATCCTGTCTCCCTT | TGG | 39.01106 | 501 |
| 2969 | 1 | TACAACTGCCAAAGGGAGAC | AGG | 46.03039 | 502 |
| 2975 | 1 | TGCCAAAGGGAGACAGGATC | TGG | 35.48964 | 503 |
| 2976 | 1 | GCCAAAGGGAGACAGGATCT | GGG | 44.90032 | 504 |
| 2977 | 1 | CCAAAGGGAGACAGGATCTG | GGG | 58.20173 | 505 |
| 2978 | 1 | CAAAGGGAGACAGGATCTGG | GGG | 63.61687 | 506 |
| 2982 | 1 | GGGAGACAGGATCTGGGGGA | AGG | 54.73323 | 507 |
| 2985 | 1 | AGACAGGATCTGGGGGAAGG | AGG | 49.86207 | 508 |
| 2998 | −1 | ATATATATATGTCTATGTGG | AGG | 65.35548 | 509 |
| 3001 | −1 | TATATATATATATGTCTATG | TGG | 58.96155 | 510 |
| 3084 | 1 | GATTCTTAATGATGATATCA | TGG | 42.04328 | 511 |
| 3110 | −1 | AAAGCTTATTATATTAATAA | TGG | 32.75644 | 512 |
| 3167 | 1 | AAGATGAAGAAGAAGAAAAC | AGG | 48.09653 | 513 |
| 3253 | 1 | ATTATTGTACTTAATTCAGC | TGG | 42.31373 | 514 |
| 3275 | −1 | TACATAATATAATATTAGCA | AGG | 60.27522 | 515 |
| 3312 | 1 | ATGAGATACACACATATATA | TGG | 43.91874 | 516 |

TABLE 5
gRNA sequences targeted for CsSP5G-2

| Position | | | | | | |
|---|---|---|---|---|---|---|
| on SEQ. | | | | | | SEQ. |
| ID. | | | | Specificity | Efficiency | ID. |
| NO: 13 | Strand | Sequence | PAM | Score | Score | NO. |
| 263 | 1 | TATAATTAATTAAGAATATA | TGG | 22.23226 | 32.43589 | 517 |
| 274 | 1 | AAGAATATATGGCTAGAGAT | AGG | 79.37203 | 45.23675 | 518 |
| 275 | 1 | AGAATATATGGCTAGAGATA | GGG | 68.00411 | 53.98613 | 519 |
| 287 | 1 | TAGAGATAGGGACCCTCTTG | TGG | 94.26857 | 52.53234 | 520 |
| 288 | −1 | TTACTCTACCAACCACAAGA | GGG | 86.20032 | 67.79474 | 521 |
| 289 | −1 | ATTACTCTACCAACCACAAG | AGG | 78.08364 | 59.18615 | 522 |
| 291 | 1 | GATAGGGACCCTCTTGTGGT | TGG | 83.63088 | 56.67036 | 523 |
| 303 | 1 | CTTGTGGTTGGTAGAGTAAT | AGG | 79.58025 | 35.27607 | 524 |
| 314 | 1 | TAGAGTAATAGGAGATGTTT | TGG | 65.94111 | 32.95839 | 525 |
| 328 | 1 | ATGTTTTGGATCCTTTTACA | AGG | 70.02417 | 55.39173 | 526 |
| 328 | −1 | AGAGAGACTGACCTTGTAAA | AGG | 61.64142 | 42.08228 | 527 |
| 354 | 1 | GTCTCTCTTAGAGTGAGTTA | TGG | 77.15915 | 47.06405 | 528 |
| 364 | 1 | GAGTGAGTTATGGTAATGAG | AGG | 88.2009 | 66.48722 | 529 |
| 375 | 1 | GGTAATGAGAGGTCAACAGA | TGG | 69.50771 | 64.33275 | 530 |
| 400 | −1 | GGTTGGTTAACAATTTGGGA | AGG | 71.89396 | 61.46045 | 531 |
| 404 | −1 | ACGAGGTTGGTTAACAATTT | GGG | 74.7603 | 32.45336 | 532 |
| 405 | −1 | CACGAGGTTGGTTAACAATT | TGG | 47.58272 | 23.81735 | 533 |
| 417 | −1 | CACCAATATCAACACGAGGT | TGG | 87.81265 | 61.80226 | 534 |
| 421 | −1 | TCACCACCAATATCAACACG | AGG | 83.79878 | 75.84355 | 535 |
| 426 | 1 | AACCAACCTCGTGTTGATAT | TGG | 89.69152 | 44.46178 | 536 |
| 429 | 1 | CAACCTCGTGTTGATATTGG | TGG | 86.47779 | 58.81318 | 537 |
| 442 | 1 | ATATTGGTGGTGATGACCTA | AGG | 85.41543 | 60.13671 | 538 |
| 447 | −1 | CCAAAGTGTAGAAGGTCCTT | AGG | 92.80423 | 42.09425 | 539 |
| 455 | −1 | TTAATTTACCAAAGTGTAGA | AGG | 72.5548 | 48.09029 | 540 |
| 458 | 1 | CCTAAGGACCTTCTACACTT | TGG | 96.23278 | 50.47553 | 541 |
| 493 | −1 | TGAAATCATTATGAATATTG | AGG | 48.22121 | 50.25272 | 542 |
| 570 | 1 | TCATATATTGAAATTATTAC | AGG | 52.69845 | 36.5369 | 543 |
| 576 | 1 | ATTGAAATTATTACAGGTCA | TGG | 77.63024 | 44.4074 | 544 |
| 579 | 1 | GAAATTATTACAGGTCATGG | TGG | 68.71918 | 67.377 | 545 |
| 585 | 1 | ATTACAGGTCATGGTGGATC | CGG | 91.58358 | 50.20677 | 546 |
| 593 | −1 | TTGCTAGGGCTAGGAGCATC | CGG | 91.68151 | 49.91113 | 547 |
| 602 | −1 | AGATTGGGGTTGCTAGGGCT | AGG | 88.79083 | 44.68519 | 548 |
| 607 | −1 | CCCTTAGATTGGGGTTGCTA | GGG | 95.67441 | 47.67473 | 549 |
| 608 | −1 | TCCCTTAGATTGGGGTTGCT | AGG | 93.93984 | 44.15699 | 550 |
| 616 | −1 | GCAAATACTCCCTTAGATTG | GGG | 90.51159 | 58.46921 | 551 |
| 617 | 1 | GCCCTAGCAACCCCAATCTA | AGG | 96.11133 | 36.0562 | 552 |
| 617 | −1 | TGCAAATACTCCCTTAGATT | GGG | 80.38169 | 30.97985 | 553 |
| 618 | 1 | CCCTAGCAACCCCAATCTAA | GGG | 91.08172 | 45.48361 | 554 |
| 618 | −1 | ATGCAAATACTCCCTTAGAT | TGG | 83.72207 | 38.79211 | 555 |
| 632 | 1 | ATCTAAGGGAGTATTTGCAT | TGG | 72.37072 | 59.37908 | 556 |
| 691 | 1 | TATTATTATTATAATAGATG | AGG | 33.94489 | 49.0571 | 557 |
| 692 | 1 | ATTATTATTATAATAGATGA | GGG | 46.17269 | 54.14818 | 558 |
| 824 | 1 | TTTAATTTTGTATAAAAATT | TGG | 30.69736 | 25.68348 | 559 |
| 941 | −1 | TTCATGCACACAACACATGT | TGG | 73.37183 | 55.60825 | 560 |
| 1002 | −1 | CAAAAGATAAAGACATATTT | TGG | 40.5511 | 14.24454 | 561 |
| 1014 | 1 | CAAAATATGTCTTTATCTTT | TGG | 49.25714 | 18.66841 | 562 |
| 1049 | 1 | CATTTTATAAAGATGTTAGT | TGG | 62.22119 | 33.17467 | 563 |
| 1050 | 1 | ATTTTATAAAGATGTTAGTT | GGG | 55.65628 | 40.20867 | 564 |
| 1235 | 1 | TTTTAGTGTCAGTTTTGAAT | TGG | 56.17701 | 29.61892 | 565 |
| 1253 | −1 | ACAAAATTCTGTAATTATTA | GGG | 40.47765 | 26.7988 | 566 |
| 1254 | −1 | TACAAAATTCTGTAATTATT | AGG | 42.0428 | 13.97206 | 567 |
| 1286 | −1 | ACAAATTAAAACAAGCTTTA | GGG | 61.75581 | 23.11439 | 568 |
| 1287 | −1 | AACAAATTAAAACAAGCTTT | AGG | 57.55848 | 32.2923 | 569 |
| 1310 | 1 | TTTAATTTGTTAAAGTGACT | AGG | 43.2 | 54.58344 | 570 |
| 1358 | −1 | AAACATGTAAAAGAATTTAA | GGG | 28.72101 | 30.13118 | 571 |
| 1359 | −1 | TAAACATGTAAAAGAATTTA | AGG | 36.55236 | 17.03894 | 572 |
| 1386 | −1 | TAGCTAGCATATATGGATTG | TGG | 85.80949 | 58.5557 | 573 |
| 1393 | −1 | ATTTATATAGCTAGCATATA | TGG | 67.09149 | 25.17453 | 574 |
| 1414 | 1 | TAGCTATATAAATATAAATA | TGG | 34.98771 | 35.14496 | 575 |
| 1419 | 1 | ATATAAATATAAATATGGAA | AGG | 38.5034 | 60.53312 | 576 |
| 1427 | 1 | ATAAATATGGAAAGGATATA | TGG | 50.85775 | 32.49959 | 577 |
| 1428 | 1 | TAAATATGGAAAGGATATAT | GGG | 55.43276 | 33.709 | 578 |
| 1476 | 1 | AAAGCTGATGAGAAAGAATG | TGG | 59.04283 | 66.42004 | 579 |
| 1481 | 1 | TGATGAGAAAGAATGTGGTT | TGG | 56.44644 | 50.94728 | 580 |
| 1482 | 1 | GATGAGAAAGAATGTGGTTT | GGG | 45.55022 | 37.02061 | 581 |
| 1483 | 1 | ATGAGAAAGAATGTGGTTTG | GGG | 32.76114 | 59.03998 | 582 |
| 1504 | 1 | GGATGAATTTTGAATGATGA | AGG | 57.96939 | 48.10583 | 583 |
| 1505 | 1 | GATGAATTTTGAATGATGAA | GGG | 51.67398 | 53.0293 | 584 |
| 1511 | 1 | TTTTGAATGATGAAGGGATG | AGG | 74.87655 | 52.46871 | 585 |
| 1523 | 1 | AAGGGATGAGGCTGTGTGTG | TGG | 91.76706 | 55.94352 | 586 |
| 1545 | −1 | GGGACATGCTATAGCTAGCA | GGG | 93.3302 | 60.76795 | 587 |
| 1546 | −1 | GGGGACATGCTATAGCTAGC | AGG | 96.3887 | 49.84652 | 588 |
| 1565 | −1 | ATTTTAATGGGGGACAAAAG | GGG | 75.18965 | 50.95202 | 589 |
| 1566 | −1 | CATTTTAATGGGGGACAAAA | GGG | 70.07484 | 28.24863 | 590 |
| 1567 | −1 | CCATTTTAATGGGGGACAAA | AGG | 72.90401 | 29.61281 | 591 |
| 1575 | −1 | TGAGGTGGCCATTTTAATGG | GGG | 88.0117 | 64.57289 | 592 |
| 1576 | −1 | GTGAGGTGGCCATTTTAATG | GGG | 88.89714 | 54.06851 | 593 |
| 1577 | −1 | TGTGAGGTGGCCATTTTAAT | GGG | 86.9284 | 20.4018 | 594 |
| 1578 | 1 | CCTTTTGTCCCCCATTAAAA | TGG | 84.04315 | 22.66327 | 595 |
| 1578 | −1 | GTGTGAGGTGGCCATTTTAA | TGG | 90.68493 | 23.47984 | 596 |
| 1590 | −1 | AAAACCTTCTTAGTGTGAGG | TGG | 86.37634 | 69.54075 | 597 |
| 1593 | −1 | GTGAAAACCTTCTTAGTGTG | AGG | 80.43362 | 60.43941 | 598 |
| 1597 | 1 | ATGGCCACCTCACACTAAGA | AGG | 96.3153 | 56.86845 | 599 |
| 1665 | 1 | ATATATACATACACATGTAT | AGG | 43.76861 | 45.18352 | 600 |
| 1666 | 1 | TATATACATACACATGTATA | GGG | 22.29849 | 55.90543 | 601 |
| 1733 | −1 | CTATGTTCGAATTCAAATTC | GGG | 59.04622 | 34.62224 | 602 |
| 1734 | −1 | TCTATGTTCGAATTCAAATT | CGG | 55.60998 | 36.42467 | 603 |
| 1756 | 1 | TTCGAACATAGACTCAGATT | TGG | 67.83184 | 35.59295 | 604 |
| 1769 | −1 | CAGGGTTCAGGGTCGAATTA | GGG | 96.49558 | 35.95472 | 605 |
| 1770 | −1 | TCAGGGTTCAGGGTCGAATT | AGG | 93.28847 | 30.0118 | 606 |
| 1780 | −1 | AGTTCGTGTTTCAGGGTTCA | GGG | 91.81519 | 43.58548 | 607 |
| 1781 | −1 | AAGTTCGTGTTTCAGGGTTC | AGG | 94.75493 | 26.7905 | 608 |
| 1787 | −1 | GAGTCTAAGTTCGTGTTTCA | GGG | 82.9438 | 35.03576 | 609 |
| 1788 | −1 | TGAGTCTAAGTTCGTGTTTC | AGG | 86.51258 | 15.64056 | 610 |
| 1806 | 1 | CACGAACTTAGACTCAGACC | TGG | 97.32676 | 55.22454 | 611 |
| 1811 | 1 | ACTTAGACTCAGACCTGGAC | TGG | 94.91925 | 50.92057 | 612 |
| 1813 | −1 | TTTGGGTCAAGGTCCAGTCC | AGG | 94.63584 | 46.64457 | 613 |
| 1824 | −1 | TCGGGTCGGGGTTTGGGTCA | AGG | 85.77517 | 38.7222 | 614 |
| 1830 | −1 | CGGGGTTCGGGTCGGGGTTT | GGG | 93.19713 | 29.12979 | 615 |
| 1831 | −1 | TCGGGGTTCGGGTCGGGGTT | TGG | 90.00364 | 16.04517 | 616 |
| 1836 | −1 | TCGAGTCGGGGTTCGGGTCG | GGG | 94.47451 | 28.40008 | 617 |
| 1837 | −1 | TTCGAGTCGGGGTTCGGGTC | GGG | 89.12695 | 28.3022 | 618 |
| 1838 | −1 | GTTCGAGTCGGGGTTCGGGT | CGG | 93.33815 | 44.74237 | 619 |
| 1842 | −1 | CGGGGTTCGAGTCGGGGTTC | GGG | 97.78823 | 33.72492 | 620 |
| 1843 | −1 | TCGGGGTTCGAGTCGGGGTT | CGG | 95.88259 | 29.847 | 621 |
| 1848 | −1 | TAGTTTCGGGGTTCGAGTCG | GGG | 89.32043 | 54.61581 | 622 |
| 1849 | −1 | CTAGTTTCGGGGTTCGAGTC | GGG | 95.12488 | 35.21533 | 623 |
| 1850 | −1 | TCTAGTTTCGGGGTTCGAGT | CGG | 89.32442 | 47.1652 | 624 |
| 1860 | −1 | CCAGGTCCAGTCTAGTTTCG | GGG | 97.39694 | 59.72421 | 625 |
| 1861 | −1 | TCCAGGTCCAGTCTAGTTTC | GGG | 95.56199 | 29.76351 | 626 |
| 1862 | −1 | GTCCAGGTCCAGTCTAGTTT | CGG | 93.75616 | 32.43822 | 627 |
| 1865 | 1 | CTCGAACCCCGAAACTAGAC | TGG | 95.01385 | 41.77216 | 628 |
| 1871 | 1 | CCCCGAAACTAGACTGGACC | TGG | 99.6993 | 44.07102 | 629 |
| 1878 | 1 | ACTAGACTGGACCTGGACTC | TGG | 98.4311 | 56.19546 | 630 |
| 1878 | −1 | TAGGTCCTAGGCCAGAGTCC | AGG | 98.14848 | 53.14281 | 631 |
| 1884 | 1 | CTGGACCTGGACTCTGGCCT | AGG | 98.33955 | 55.36929 | 632 |
| 1890 | −1 | CTAGACCCGAGCTAGGTCCT | AGG | 99.08664 | 55.22238 | 633 |
| 1895 | 1 | CTCTGGCCTAGGACCTAGCT | CGG | 97.78611 | 44.89925 | 634 |
| 1896 | 1 | TCTGGCCTAGGACCTAGCTC | GGG | 98.32979 | 55.94427 | 635 |
| 1897 | −1 | GCCTGACCTAGACCCGAGCT | AGG | 99.73282 | 62.86618 | 636 |
| 1902 | 1 | CTAGGACCTAGCTCGGGTCT | AGG | 99.47133 | 48.45849 | 637 |
| 1907 | 1 | ACCTAGCTCGGGTCTAGGTC | AGG | 98.95846 | 46.63816 | 638 |
| 1914 | 1 | TCGGGTCTAGGTCAGGCGTC | CGG | 98.55442 | 40.11313 | 639 |
| 1915 | 1 | CGGGTCTAGGTCAGGCGTCC | GGG | 99.96756 | 43.65285 | 640 |
| 1922 | 1 | AGGTCAGGCGTCCGGGTCCG | AGG | 99.22245 | 53.42575 | 641 |
| 1922 | −1 | CCCAGATCTGACCTCGGACC | CGG | 99.30512 | 55.96585 | 642 |
| 1928 | −1 | CACGAACCCAGATCTGACCT | CGG | 96.77532 | 68.87557 | 643 |
| 1932 | 1 | TCCGGGTCCGAGGTCAGATC | TGG | 99.24263 | 31.05262 | 644 |
| 1933 | 1 | CCGGGTCCGAGGTCAGATCT | GGG | 97.78879 | 47.515 | 645 |
| 1945 | 1 | TCAGATCTGGGTTCGTGTTC | TGG | 98.00119 | 23.41796 | 646 |
| 1946 | 1 | CAGATCTGGGTTCGTGTTCT | GGG | 90.6576 | 34.46177 | 647 |
| 1947 | 1 | AGATCTGGGTTCGTGTTCTG | GGG | 72.74378 | 61.20728 | 648 |
| 1953 | 1 | GGGTTCGTGTTCTGGGGTTC | AGG | 96.0542 | 32.84982 | 649 |
| 1957 | 1 | TCGTGTTCTGGGGTTCAGGT | TGG | 96.79548 | 32.94333 | 650 |
| 1958 | 1 | CGTGTTCTGGGGTTCAGGTT | GGG | 97.29827 | 37.64639 | 651 |
| 1962 | 1 | TTCTGGGGTTCAGGTTGGGT | TGG | 95.13759 | 36.37666 | 652 |
| 1963 | 1 | TCTGGGGTTCAGGTTGGGTT | GGG | 88.17509 | 39.38005 | 653 |
| 1968 | 1 | GGTTCAGGTTGGGTTGGGTC | TGG | 80.71071 | 33.59531 | 654 |
| 1975 | 1 | GTTGGGTTGGGTCTGGAGTC | TGG | 89.11478 | 19.45666 | 655 |
| 1976 | 1 | TTGGGTTGGGTCTGGAGTCT | GGG | 91.69313 | 39.24034 | 656 |
| 1982 | 1 | TGGGTCTGGAGTCTGGGTCT | AGG | 93.64853 | 26.29809 | 657 |
| 1983 | 1 | GGGTCTGGAGTCTGGGTCTA | GGG | 95.12459 | 46.06491 | 658 |
| 1995 | 1 | TGGGTCTAGGGTCCAGATTC | AGG | 81.74099 | 36.11531 | 659 |
| 1996 | −1 | CCTGAACCCGATCCTGAATC | TGG | 95.331 | 41.73702 | 660 |
| 2000 | 1 | CTAGGGTCCAGATTCAGGAT | CGG | 92.37173 | 53.25207 | 661 |
| 2001 | 1 | TAGGGTCCAGATTCAGGATC | GGG | 89.64241 | 49.77329 | 662 |
| 2007 | 1 | CCAGATTCAGGATCGGGTTC | AGG | 95.93196 | 32.80081 | 663 |
| 2013 | 1 | TCAGGATCGGGTTCAGGTTA | AGG | 87.67606 | 33.91225 | 664 |
| 2031 | 1 | TAAGGTTTGAGTCTGAGTCC | AGG | 94.41411 | 59.59865 | 665 |
| 2037 | 1 | TTGAGTCTGAGTCCAGGTAT | AGG | 90.1772 | 52.86993 | 666 |
| 2038 | −1 | TCCCGACCAGAACCTATACC | TGG | 87.92881 | 43.98773 | 667 |
| 2043 | 1 | CTGAGTCCAGGTATAGGTTC | TGG | 92.36672 | 45.95561 | 668 |
| 2047 | 1 | GTCCAGGTATAGGTTCTGGT | CGG | 94.39855 | 54.99259 | 669 |
| 2048 | 1 | TCCAGGTATAGGTTCTGGTC | GGG | 94.37527 | 55.16665 | 670 |
| 2075 | 1 | GAGTTCAGAGTTTGAATTCA | AGG | 63.71772 | 35.78178 | 671 |
| 2085 | 1 | TTTGAATTCAAGGTCCAATT | TGG | 68.36156 | 30.63103 | 672 |
| 2088 | −1 | GAACTCATCCAACTCCAAAT | TGG | 63.82882 | 38.88806 | 673 |
| 2091 | 1 | TTCAAGGTCCAATTTGGAGT | TGG | 83.895 | 43.86757 | 674 |
| 2108 | 1 | AGTTGGATGAGTTCATGTCA | TGG | 82.12315 | 67.34083 | 675 |
| 2174 | −1 | TTTAAAATTTTAATAGTGTT | TGG | 46.71727 | 34.83327 | 676 |
| 2291 | 1 | TTCATAATTTTTAAAATTAG | AGG | 22.81635 | 37.38885 | 677 |
| 2292 | 1 | TCATAATTTTTAAAATTAGA | GGG | 36.07909 | 41.07827 | 678 |
| 2308 | −1 | TATTTTTATCTTTACTTATA | GGG | 47.52393 | 23.50322 | 679 |
| 2309 | −1 | TTATTTTTATCTTTACTTAT | AGG | 46.63947 | 16.34323 | 680 |
| 2421 | −1 | TTTACTGTACCGAATATTCA | CGG | 79.17329 | 41.6042 | 681 |
| 2423 | 1 | TTGTAGTTACCGTGAATATT | CGG | 84.39107 | 32.91515 | 682 |
| 2438 | 1 | ATATTCGGTACAGTAAATTA | AGG | 78.81327 | 39.9063 | 683 |
| 2442 | 1 | TCGGTACAGTAAATTAAGGA | TGG | 89.21293 | 57.49355 | 684 |
| 2523 | −1 | ATATATAAAAATATAAATTG | TGG | 25.27378 | 59.76844 | 685 |
| 2552 | 1 | TATATTATTAATCTAGATAA | TGG | 50.26283 | 44.83698 | 686 |
| 2604 | −1 | TTATAATTATACTAATATAT | AGG | 35.57578 | 34.45299 | 687 |
| 2632 | 1 | TTATAATAATTATACATGTT | TGG | 44.45843 | 35.08265 | 688 |
| 2648 | 1 | TGTTTGGCAATTTCAATTTT | AGG | 48.67666 | 19.55566 | 689 |
| 2652 | 1 | TGGCAATTTCAATTTTAGGT | TGG | 62.81544 | 45.19466 | 690 |
| 2668 | 1 | AGGTTGGTGACTGATATTCC | TGG | 90.67651 | 39.09227 | 691 |
| 2675 | −1 | AAGCTTGGCCCGGTAGTTCC | AGG | 100 | 41.23058 | 692 |
| 2677 | 1 | ACTGATATTCCTGGAACTAC | CGG | 83.0177 | 48.93325 | 693 |
| 2678 | 1 | CTGATATTCCTGGAACTACC | GGG | 96.95467 | 62.10752 | 694 |
| 2685 | −1 | CGGCTCACCGAAGCTTGGCC | CGG | 98.17534 | 48.9322 | 695 |
| 2689 | 1 | GGAACTACCGGGCCAAGCTT | CGG | 98.10529 | 46.22153 | 696 |
| 2690 | −1 | ATGAACGGCTCACCGAAGCT | TGG | 97.02801 | 59.46928 | 697 |
| 2705 | −1 | GTATTATTATGAAGTATGAA | CGG | 54.7198 | 57.67305 | 698 |
| 2776 | 1 | ACGCTGTAAACAAAATAGTG | CGG | 81.23666 | 66.20102 | 699 |
| 2870 | 1 | ATTAATTGTTTATTATGTGT | AGG | 37.44309 | 49.28958 | 700 |
| 2878 | 1 | TTTATTATGTGTAGGACAAG | AGG | 75.38758 | 55.5504 | 701 |
| 2881 | 1 | ATTATGTGTAGGACAAGAGG | TGG | 76.37859 | 65.45521 | 702 |
| 2901 | 1 | TGGTGTGCTACGAGAACCCG | CGG | 98.37073 | 69.35789 | 703 |
| 2905 | 1 | GTGCTACGAGAACCCGCGGC | CGG | 100 | 49.67584 | 704 |
| 2906 | 1 | TGCTACGAGAACCCGCGGCC | GGG | 99.48738 | 51.94565 | 705 |
| 2906 | −1 | ATGAATCCCCACCCGGCCGC | GGG | 99.3641 | 52.12455 | 706 |
| 2907 | −1 | GATGAATCCCCACCCGGCCG | CGG | 99.86331 | 48.71998 | 707 |
| 2909 | 1 | TACGAGAACCCGCGGCCGGG | TGG | 99.98179 | 55.19917 | 708 |
| 2910 | 1 | ACGAGAACCCGCGGCCGGGT | GGG | 100 | 48.89766 | 709 |
| 2911 | 1 | CGAGAACCCGCGGCCGGGTG | GGG | 99.8583 | 44.45536 | 710 |
| 2913 | −1 | CATACCGATGAATCCCCACC | CGG | 91.97498 | 55.96558 | 711 |
| 2920 | 1 | GCGGCCGGGTGGGGATTCAT | CGG | 96.23188 | 49.33169 | 712 |
| 2941 | 1 | GGTATGTATTTGTGTTGTTC | CGG | 49.44229 | 42.62051 | 713 |
| 2948 | 1 | ATTTGTGTTGTTCCGGCAAT | TGG | 93.12907 | 33.46831 | 714 |
| 2949 | 1 | TTTGTGTTGTTCCGGCAATT | GGG | 95.02167 | 46.97766 | 715 |
| 2949 | −1 | CCGTTTGCCTTCCCAATTGC | CGG | 95.00883 | 39.46262 | 716 |
| 2953 | 1 | TGTTGTTCCGGCAATTGGGA | AGG | 88.65091 | 55.97024 | 717 |
| 2960 | 1 | CCGGCAATTGGGAAGGCAAA | CGG | 93.92485 | 54.12282 | 718 |
| 2972 | 1 | AAGGCAAACGGTGTTCGCGC | CGG | 99.47463 | 41.65064 | 719 |
| 2973 | 1 | AGGCAAACGGTGTTCGCGCC | GGG | 99.96331 | 37.60685 | 720 |
| 2974 | 1 | GGCAAACGGTGTTCGCGCCG | GGG | 99.57812 | 56.96599 | 721 |
| 2977 | 1 | AAACGGTGTTCGCGCCGGGG | TGG | 99.29854 | 55.4399 | 722 |
| 2980 | −1 | TTGAAGTTCTGACGCCACCC | CGG | 97.94034 | 59.80253 | 723 |
| 3005 | −1 | ATAAAGCTCAGCAAAGTCTT | TGG | 76.47984 | 43.946 | 724 |
| 3024 | 1 | TTTGCTGAGCTTTATAACCT | TGG | 70.03213 | 44.20648 | 725 |
| 3030 | −1 | GGGCAGCAACAGGCAAACCA | AGG | 96.32764 | 68.3844 | 726 |
| 3040 | −1 | TTGTAATAAAGGGCAGCAAC | AGG | 93.04706 | 46.09672 | 727 |
| 3050 | −1 | CCTTTGGCAGTTGTAATAAA | GGG | 84.67114 | 32.56095 | 728 |
| 3051 | −1 | CCCTTTGGCAGTTGTAATAA | AGG | 86.81624 | 29.14878 | 729 |
| 3061 | 1 | CCCTTTATTACAACTGCCAA | AGG | 95.16301 | 54.00419 | 730 |
| 3062 | 1 | CCTTTATTACAACTGCCAAA | GGG | 87.58701 | 58.42829 | 731 |
| 3066 | −1 | CCCCAGATCCTGTCTCCCTT | TGG | 97.97572 | 39.01106 | 732 |
| 3069 | 1 | TACAACTGCCAAAGGGAGAC | AGG | 93.01226 | 46.03039 | 733 |
| 3075 | 1 | TGCCAAAGGGAGACAGGATC | TGG | 98.02303 | 35.48964 | 734 |
| 3076 | 1 | GCCAAAGGGAGACAGGATCT | GGG | 98.25717 | 44.90032 | 735 |
| 3077 | 1 | CCAAAGGGAGACAGGATCTG | GGG | 94.1697 | 58.20173 | 736 |
| 3078 | 1 | CAAAGGGAGACAGGATCTGG | GGG | 96.85202 | 63.61687 | 737 |
| 3082 | 1 | GGGAGACAGGATCTGGGGGA | AGG | 98.34396 | 54.73323 | 738 |
| 3085 | 1 | AGACAGGATCTGGGGGAAGG | AGG | 95.84931 | 49.86207 | 739 |
| 3098 | −1 | ATATATATATGTCTATGTGG | AGG | 57.02276 | 65.35548 | 740 |
| 3101 | −1 | TATATATATATATGTCTATG | TGG | 42.14695 | 58.96155 | 741 |
| 3185 | 1 | GATTCTTAATGATGATATCA | TGG | 35.38161 | 43.13298 | 742 |
| 3213 | −1 | AAAGCTTATTATATTAATAA | TGG | 35.17605 | 32.75644 | 743 |
| 3250 | 1 | TATATATATATAGAATAAGA | TGG | 45.50324 | 48.36613 | 744 |
| 3261 | 1 | AGAATAAGATGGAAGAAAAC | AGG | 48.6601 | 44.82802 | 745 |

TABLE 6
gRNA sequences targeted for CsSP5G-3

| Position | | | | | |
|---|---|---|---|---|---|
| on SEQ. | | | | | |
| ID. | | | | Efficiency | SEQ. ID. |
| NO: 16 | Strand | Sequence | PAM | Score | NO. |
| 1007 | −1 | AACTTGGCTGTGGTGGAAGG | AGG | 62.58111 | 746 |
| 1010 | −1 | AGGAACTTGGCTGTGGTGGA | AGG | 53.35214 | 747 |
| 1014 | −1 | CAAAAGGAACTTGGCTGTGG | TGG | 59.7649 | 748 |
| 1017 | −1 | TGCCAAAAGGAACTTGGCTG | TGG | 49.9405 | 749 |
| 1023 | −1 | TTCAACTGCCAAAAGGAACT | TGG | 48.07818 | 750 |
| 1026 | 1 | CACCACAGCCAAGTTCCTTT | TGG | 32.23959 | 751 |
| 1030 | −1 | CGTTTACTTCAACTGCCAAA | AGG | 54.51517 | 752 |
| 1052 | 1 | TTGAAGTAAACGACAGCAAC | AGG | 41.20281 | 753 |
| 1062 | 1 | CGACAGCAACAGGCAATCCA | AGG | 59.31913 | 754 |
| 1068 | −1 | TTTGCTGAGATCTACAACCT | TGG | 39.26965 | 755 |
| 1112 | 1 | TTGAAGTTATGTCGCCACCC | AGG | 64.35662 | 756 |
| 1115 | −1 | AGACAGTGTATGCACCTGGG | TGG | 69.83127 | 757 |
| 1118 | −1 | GGCAGACAGTGTATGCACCT | GGG | 69.43409 | 758 |
| 1119 | −1 | AGGCAGACAGTGTATGCACC | TGG | 46.20011 | 759 |
| 1139 | −1 | TTCTGTTTCGACAGTTAGGA | AGG | 47.13328 | 760 |
| 1143 | −1 | TTTGTTCTGTTTCGACAGTT | AGG | 49.53212 | 761 |
| 1181 | 1 | TAGCGATGTATTCCCACCGT | CGG | 68.69506 | 762 |
| 1182 | −1 | GAGAGCCCTAGACCGACGGT | GGG | 72.28535 | 763 |
| 1183 | −1 | CGAGAGCCCTAGACCGACGG | TGG | 62.48353 | 764 |
| 1186 | −1 | CTACGAGAGCCCTAGACCGA | CGG | 67.63038 | 765 |
| 1187 | 1 | TGTATTCCCACCGTCGGTCT | AGG | 37.13047 | 766 |
| 1188 | 1 | GTATTCCCACCGTCGGTCTA | GGG | 52.6053 | 767 |
| 1224 | −1 | GTGTATATACGCATATATGT | AGG | 53.19418 | 768 |
| 1367 | 1 | TTAACTTATCGTTAACTTAT | TGG | 21.66841 | 769 |
| 1452 | 1 | AGTGTATTAATATGTACCAA | AGG | 64.2117 | 770 |
| 1457 | −1 | GCAACTACGGGAACAGCCTT | TGG | 43.45249 | 771 |
| 1469 | −1 | ACTGATATTCCCGCAACTAC | GGG | 56.57771 | 772 |
| 1470 | 1 | AAAGGCTGTTCCCGTAGTTG | CGG | 54.3468 | 773 |
| 1470 | −1 | GACTGATATTCCCGCAACTA | CGG | 47.03522 | 774 |
| 1471 | 1 | AAGGCTGTTCCCGTAGTTGC | GGG | 43.75953 | 775 |
| 1494 | −1 | AGTGTATAACTTTTTCAGGT | TGG | 45.74911 | 776 |
| 1498 | −1 | TTTAAGTGTATAACTTTTTC | AGG | 12.25519 | 777 |
| 1553 | 1 | ATAAAATTAATAATTATTAG | TGG | 40.90627 | 778 |
| 1569 | 1 | TTAGTGGCACACACTATTGA | TGG | 50.84478 | 779 |
| 1583 | −1 | AATATTACTAACGAATGACA | AGG | 71.2185 | 780 |
| 1633 | −1 | TGTACTTAGTAATATCATTT | CGG | 39.17215 | 781 |
| 1658 | −1 | ATCTTAAAGAATATTTGCAT | TGG | 51.43028 | 782 |
| 1673 | 1 | TGCAAATATTCTTTAAGATT | AGG | 33.25661 | 783 |
| 1682 | 1 | TCTTTAAGATTAGGTTCGCT | AGG | 47.68808 | 784 |
| 1683 | 1 | CTTTAAGATTAGGTTCGCTA | GGG | 53.86326 | 785 |
| 1688 | 1 | AGATTAGGTTCGCTAGGGTT | AGG | 44.74391 | 786 |
| 1713 | −1 | ACTGTTAATGTTGCAGGTTA | TGG | 38.16734 | 787 |
| 1719 | −1 | ATTAATACTGTTAATGTTGC | AGG | 25.17433 | 788 |
| 1839 | 1 | CGTATACTTAATTTTATGCT | AGG | 51.56297 | 789 |
| 1980 | 1 | TATTTGTGCAATTATAAGTT | AGG | 41.35824 | 790 |
| 1992 | −1 | GTCAAAGACCCACAACAATA | AGG | 47.19197 | 791 |
| 1994 | 1 | TAAGTTAGGCCTTATTGTTG | TGG | 52.99944 | 792 |
| 1995 | 1 | AAGTTAGGCCTTATTGTTGT | GGG | 35.64647 | 793 |
| 2051 | 1 | TTAACAGTGTTAATTTTAAA | CGG | 27.14498 | 794 |
| 2196 | −1 | TTGGTTTATGAAAAATTAGT | GGG | 43.4587 | 795 |
| 2197 | −1 | CTTGGTTTATGAAAAATTAG | TGG | 50.90688 | 796 |
| 2215 | −1 | TGCCTATATAACTAAATTCT | TGG | 33.54393 | 797 |
| 2224 | 1 | AACCAAGAATTTAGTTATAT | AGG | 34.54892 | 798 |
| 2274 | −1 | TCTCAGGACTTTCTACACTT | TGG | 48.01676 | 799 |
| 2290 | −1 | AGATTGGTGGAGATGATCTC | AGG | 51.3529 | 800 |
| 2303 | −1 | TCACCTAGGGTTGAGATTGG | TGG | 50.45115 | 801 |
| 2306 | −1 | AACTCACCTAGGGTTGAGAT | TGG | 52.86728 | 802 |
| 2311 | 1 | TCTCCACCAATCTCAACCCT | AGG | 61.53779 | 803 |
| 2316 | −1 | TCAAGTTGCTAACTCACCTA | GGG | 50.65517 | 804 |
| 2317 | −1 | CTCAAGTTGCTAACTCACCT | AGG | 56.33574 | 805 |
| 2332 | 1 | GGTGAGTTAGCAACTTGAGA | AGG | 55.71603 | 806 |
| 2339 | 1 | TAGCAACTTGAGAAGGTCTA | AGG | 50.2432 | 807 |
| 2357 | −1 | GGGATTAGGGCTATTAACAA | TGG | 57.57407 | 808 |
| 2370 | −1 | AGTATCATATAGTGGGATTA | GGG | 45.56512 | 809 |
| 2371 | −1 | AAGTATCATATAGTGGGATT | AGG | 31.11638 | 810 |
| 2377 | −1 | CTCTTAAAGTATCATATAGT | GGG | 47.53587 | 811 |
| 2378 | −1 | TCTCTTAAAGTATCATATAG | TGG | 49.25586 | 812 |
| 2407 | 1 | AGAGAGACAGTTTTTGTGAA | AGG | 50.90759 | 813 |
| 2408 | 1 | GAGAGACAGTTTTTGTGAAA | GGG | 39.7937 | 814 |
| 2421 | −1 | GAGAGTGATAAGTGATGTGT | TGG | 59.68802 | 815 |
| 2443 | −1 | ATAGGGATCCTCTTGTGGTT | GGG | 49.32786 | 816 |
| 2444 | −1 | AATAGGGATCCTCTTGTGGT | TGG | 40.13389 | 817 |
| 2446 | 1 | ATCACTCTCCCAACCACAAG | AGG | 54.01665 | 818 |
| 2448 | −1 | GGCTAATAGGGATCCTCTTG | TGG | 57.16178 | 819 |
| 2460 | −1 | ATATATATAAATGGCTAATA | GGG | 36.81696 | 820 |
| 2461 | −1 | TATATATATAAATGGCTAAT | AGG | 23.14055 | 821 |
| 2469 | −1 | ATAGTTGTTATATATATAAA | TGG | 34.2573 | 822 |
| 2520 | 1 | AGAGAGATAGAGAGAAGAAG | AGG | 53.49955 | 823 |
| 2521 | 1 | GAGAGATAGAGAGAAGAAGA | GGG | 63.65805 | 824 |
| 2537 | 1 | AAGAGGGTTTGATGAGTTTT | TGG | 25.7462 | 825 |
| 2550 | 1 | GAGTTTTTGGTTGTATAATT | TGG | 28.78721 | 826 |
| 2553 | 1 | TTTTTGGTTGTATAATTTGG | TGG | 50.48919 | 827 |
| 2574 | 1 | GGCTGACATTCAACAATTTA | TGG | 15.68184 | 828 |

TABLE 7
gRNA sequences targeted for CsSP5G-4

| Position | | | | | |
|---|---|---|---|---|---|
| on SEQ. | | | | | |
| ID. | | | | Efficiency | SEQ. ID. |
| NO: 19 | Strand | Sequence | PAM | Score | NO. |
| 732 | −1 | TAAAGTTATTGGGAGTTGTG | TGG | 66.8211 | 829 |
| 742 | −1 | CCGACTACTGTAAAGTTATT | GGG | 28.42176 | 830 |
| 743 | −1 | GCCGACTACTGTAAAGTTAT | TGG | 36.02326 | 831 |
| 753 | 1 | CCCAATAACTTTACAGTAGT | CGG | 40.53914 | 832 |
| 765 | −1 | TTATATAATTCTTATAGCAA | TGG | 48.27692 | 833 |
| 819 | −1 | AAAGGTTCTAATATATATTG | TGG | 47.44654 | 834 |
| 837 | −1 | TTAGCTTTTGTAACATCAAA | AGG | 33.39633 | 835 |
| 893 | −1 | GGCTGACATTCAACAATTTA | TGG | 15.68184 | 836 |
| 914 | −1 | GTTTTGGTTATATAATTTGG | TGG | 57.11172 | 837 |
| 917 | −1 | TGAGTTTTGGTTATATAATT | TGG | 28.40639 | 838 |
| 930 | −1 | GAAGAGGGTTTGATGAGTTT | TGG | 34.58611 | 839 |
| 945 | −1 | TGTAGAGAGAGATCAGAAGA | GGG | 60.53333 | 840 |
| 946 | −1 | ATGTAGAGAGAGATCAGAAG | AGG | 54.06301 | 841 |
| 990 | 1 | ATAGTTGTTATATATATAAA | TGG | 33.58298 | 842 |
| 998 | 1 | TATATATATAAATGGCTAAT | AGG | 23.14055 | 843 |
| 999 | 1 | ATATATATAAATGGCTAATA | GGG | 36.81696 | 844 |
| 1011 | 1 | GGCTAATAGGGATCCTCTTG | TGG | 57.16178 | 845 |
| 1013 | −1 | ATCACTCTCCCAACCACAAG | AGG | 54.01665 | 846 |
| 1015 | 1 | AATAGGGATCCTCTTGTGGT | TGG | 40.13389 | 847 |
| 1016 | 1 | ATAGGGATCCTCTTGTGGTT | GGG | 49.32786 | 848 |
| 1038 | 1 | GAGAGTGATAAGTGATGTGT | TGG | 59.68802 | 849 |
| 1051 | −1 | GAGACACAGTTTTTGTGAAA | GGG | 37.91168 | 850 |
| 1052 | −1 | AGAGACACAGTTTTTGTGAA | AGG | 52.69581 | 851 |
| 1081 | 1 | TCTCTTAAAGTATCATATAG | TGG | 51.27177 | 852 |
| 1082 | 1 | CTCTTAAAGTATCATATAGT | GGG | 49.44647 | 853 |
| 1088 | 1 | AAGTATCATATAGTGGGAAT | AGG | 36.1639 | 854 |
| 1089 | 1 | AGTATCATATAGTGGGAATA | GGG | 49.45047 | 855 |
| 1102 | 1 | GGGAATAGGGCTATTAACAA | TGG | 57.4132 | 856 |
| 1120 | −1 | TAGCAACTTGAGAAGGTCTA | AGG | 50.2432 | 857 |
| 1127 | −1 | GGTGAGTTAGCAACTTGAGA | AGG | 55.71603 | 858 |
| 1142 | 1 | CTCAAGTTGCTAACTCACCT | AGG | 56.33574 | 859 |
| 1143 | 1 | TCAAGTTGCTAACTCACCTA | GGG | 50.65517 | 860 |
| 1148 | −1 | TCTCCACCAATCTCAACCCT | AGG | 61.53779 | 861 |
| 1153 | 1 | AACTCACCTAGGGTTGAGAT | TGG | 52.86728 | 862 |
| 1156 | 1 | TCACCTAGGGTTGAGATTGG | TGG | 50.45115 | 863 |
| 1169 | 1 | AGATTGGTGGAGATGATCTC | AGG | 51.3529 | 864 |
| 1185 | 1 | TCTCAGGACTTTCTACACTT | TGG | 48.01676 | 865 |
| 1268 | 1 | ATTAATACTGTTAATGTTGC | AGG | 25.17433 | 866 |
| 1274 | 1 | ACTGTTAATGTTGCAGGTTA | TGG | 38.16734 | 867 |
| 1291 | −1 | TCGCTAGGGTTAGGAGCATC | AGG | 47.478 | 868 |
| 1300 | −1 | AGATTAGGTTCGCTAGGGTT | AGG | 44.74391 | 869 |
| 1305 | −1 | CTTTAAGATTAGGTTCGCTA | GGG | 53.86326 | 870 |
| 1306 | −1 | TCTTTAAGATTAGGTTCGCT | AGG | 47.68808 | 871 |
| 1315 | −1 | TGCAAATATTCTTTAAGATT | AGG | 33.25661 | 872 |
| 1330 | 1 | ATCTTAAAGAATATTTGCAT | TGG | 51.43028 | 873 |
| 1402 | 1 | AACTTTTAGATATATTACTT | AGG | 42.90897 | 874 |
| 1412 | 1 | TATATTACTTAGGAATCACA | AGG | 71.76671 | 875 |
| 1426 | −1 | TAGTGGCACACACTTATTGA | TGG | 44.48106 | 876 |
| 1443 | −1 | TAAAAATTAATAATTATTAG | TGG | 38.53536 | 877 |
| 1503 | 1 | TTAAAGTGTATAATTTTGTC | AGG | 40.17797 | 878 |
| 1507 | 1 | AGTGTATAATTTTGTCAGGT | TGG | 38.69861 | 879 |
| 1530 | −1 | AAGCCTGTTCCCGTAGTTGC | GGG | 43.75953 | 880 |
| 1531 | 1 | GACTGATATTCCCGCAACTA | CGG | 47.03522 | 881 |
| 1531 | −1 | AAAGCCTGTTCCCGTAGTTG | CGG | 53.51908 | 882 |
| 1532 | 1 | ACTGATATTCCCGCAACTAC | GGG | 56.57771 | 883 |
| 1538 | 1 | ATTCCCGCAACTACGGGAAC | AGG | 40.62686 | 884 |
| 1544 | 1 | GCAACTACGGGAACAGGCTT | TGG | 40.90731 | 885 |
| 1634 | −1 | TTAACTTACCATTAACTTAT | TGG | 23.66032 | 886 |
| 1637 | 1 | AAATAACACCAATAAGTTAA | TGG | 36.79107 | 887 |
| 1740 | 1 | TGTGTGTGATGATTTAATGA | TGG | 55.89947 | 888 |
| 1741 | 1 | GTGTGTGATGATTTAATGAT | GGG | 53.71776 | 889 |
| 1759 | 1 | ATGGGCGTACGCATATATGT | AGG | 58.24576 | 890 |
| 1795 | −1 | GAATTCCCACCGTCGGTCTT | GGG | 43.57541 | 891 |
| 1796 | −1 | TGAATTCCCACCGTCGGTCT | TGG | 31.13209 | 892 |
| 1797 | 1 | CTACGAGAGCCCAAGACCGA | CGG | 67.05847 | 893 |
| 1800 | 1 | CGAGAGCCCAAGACCGACGG | TGG | 58.98627 | 894 |
| 1801 | 1 | GAGAGCCCAAGACCGACGGT | GGG | 70.56006 | 895 |
| 1802 | −1 | AAGCGATGAATTCCCACCGT | CGG | 68.48212 | 896 |
| 1840 | 1 | TTTGTTCTGTTTCGACAGTT | AGG | 49.53212 | 897 |
| 1844 | 1 | TTCTGTTTCGACAGTTAGGA | AGG | 47.13328 | 898 |
| 1864 | 1 | AGGCAGACAGTGTATGCACC | CGG | 53.95754 | 899 |
| 1865 | 1 | GGCAGACAGTGTATGCACCC | GGG | 67.05624 | 900 |
| 1868 | 1 | AGACAGTGTATGCACCCGGG | TGG | 71.26007 | 901 |
| 1871 | −1 | TTGAAGTTATGTCGCCACCC | GGG | 64.36734 | 902 |
| 1872 | −1 | GTTGAAGTTATGTCGCCACC | CGG | 57.40751 | 903 |
| 1915 | 1 | TTTGCTGAAATCTACAACCT | TGG | 36.83098 | 904 |
| 1921 | −1 | CGGCAGCAACAGGCAATCCA | AGG | 56.65812 | 905 |
| 1931 | −1 | TTGAAGTAAACGGCAGCAAC | AGG | 42.77031 | 906 |
| 1941 | −1 | CTTTTGGCAGTTGAAGTAAA | CGG | 46.14269 | 907 |
| 1953 | 1 | CGTTTACTTCAACTGCCAAA | AGG | 54.51517 | 908 |
| 1957 | −1 | CACCACAGCCAAGTTCCTTT | TGG | 32.23959 | 909 |
| 1960 | 1 | TTCAACTGCCAAAAGGAACT | TGG | 48.07818 | 910 |
| 1966 | 1 | TGCCAAAAGGAACTTGGCTG | TGG | 49.9405 | 911 |
| 1969 | 1 | CAAAAGGAACTTGGCTGTGG | TGG | 59.7649 | 912 |
| 1973 | 1 | AGGAACTTGGCTGTGGTGGA | AGG | 53.35214 | 913 |
| 1976 | 1 | AACTTGGCTGTGGTGGAAGG | AGG | 62.58111 | 914 |
| 2046 | −1 | GACATATATAGATAGATAGA | TGG | 51.68794 | 915 |
| 2351 | 1 | TGTATCATCAATAATATATA | TGG | 32.79628 | 916 |

Reference is made to Table 8 presenting a summary of the sequences within the scope of the current invention.

TABLE 8
Summary of sequences within the scope of the present invention

| Sequence type | CsSP-1 | CsSP-2 | CsSP-3 | CsSP5G-1 | CsSP5G-2 | CsSP5G-3 | CsSP5G-4 |
|---|---|---|---|---|---|---|---|
| Genomic | SEQ ID | SEQ ID | SEQ ID | SEQ ID | SEQ ID | SEQ ID | SEQ ID |
| sequences | NO: 1 | NO: 4 | NO: 7 | NO: 10 | NO: 13 | NO: 16 | NO: 19 |
| Coding sequences | SEQ ID | SEQ ID | SEQ ID | SEQ ID | SEQ ID | SEQ ID | SEQ ID |
| (CDS) | NO: 2 | NO: 5 | NO: 8 | NO: 11 | NO: 14 | NO: 17 | NO: 20 |
| Amino acid | SEQ ID | SEQ ID | SEQ ID | SEQ ID | SEQ ID | SEQ ID | SEQ ID |
| sequences | NO: 3 | NO: 6 | NO: 9 | NO: 12 | NO: 15 | NO: 18 | NO: 21 |
| gRNA sequences | SEQ ID | SEQ ID | SEQ ID | SEQ ID | SEQ ID | SEQ ID | SEQ ID |
| | NO: 22- | NO: 127- | NO: 212- | NO: 284- | NO: 517- | NO: 746- | NO: 829- |
| | SEQ ID | SEQ ID | SEQ ID | SEQ ID | SEQ ID | SEQ ID | SEQ ID |
| | NO: 126 | NO: 211 | NO: 283 | NO: 516 | NO: 745 | NO: 828 | NO: 916 |
| | (Table 1) | (Table 2) | (Table 3) | (Table 4) | (Table 5) | (Table 6) | (Table 7) |

The above gRNA molecules have been cloned into suitable vectors and their sequence has been verified. In addition different Cas9 versions have been analyzed for optimal compatibility between the Cas9 protein activity and the gRNA molecule in the Cannabis plant.

The efficiency of the designed gRNA molecules have been validated by transiently transforming Cannabis tissue culture. A plasmid carrying a gRNA sequence together with the Cas9 gene has been transformed into Cannabis protoplasts. The protoplast cells have been grown for a short period of time and then were analyzed for existence of genome editing events. The positive constructs have been subjected to the herein established stable transformation protocol into Cannabis plant tissue for producing genome edited Cannabis plants in SP and/or SP5G genes.

Stage 3: Transforming Cannabis plants using Agrobacterium or biolistics (gene gun) methods. For Agrobacterium and bioloistics, a DNA plasmid carrying (Cas9 +gene specific gRNA) can be used. A vector containing a selection marker, Cas9 gene and relevant gene specific gRNA's is constructed. For biolistics, Ribonucleoprotein (RNP) complexes carrying (Cas9 protein +gene specific gRNA) are used. RNP complexes are created by mixing the Cas9 protein with relevant gene specific gRNA's.

According to some embodiments of the present invention, transformation of various Cannabis tissues was performed using particle bombardment of:
DNA vectors
Ribonucleoprotein complex (RNP's)

According to further embodiments of the present invention, transformation of various Cannabis tissues was performed using Agrobacterium (Agrobacterium tumefaciens) by:
Regeneration-based transformation
Floral-dip transformation
Seedling transformation

Transformation efficiency by A. tumefaciens has been compared to the bombardment method by transient GUS transformation experiment. After transformation, GUS staining of the transformants has been performed.

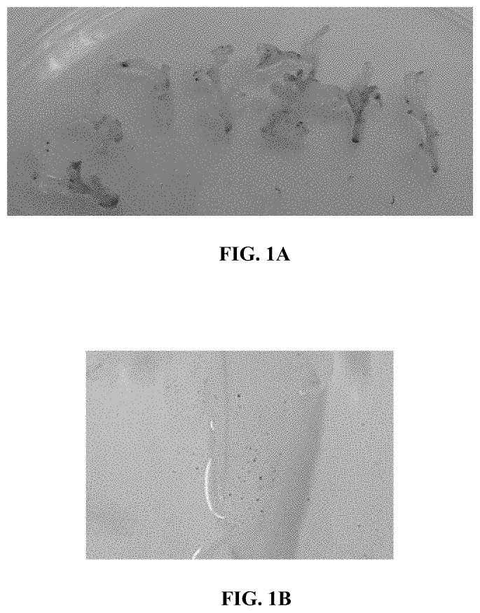

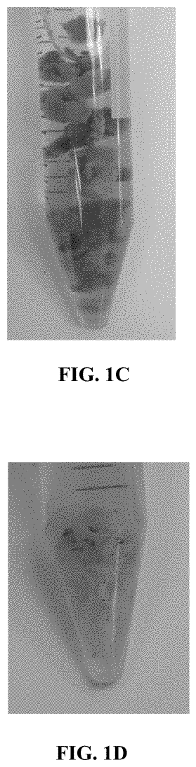

Reference is now made to FIG. 1 photographically presenting GUS staining after transient transformation of the following Cannabis tissues (A) axillary buds (B) leaf (C) calli, and (D) cotyledons.

FIG. 1 demonstrates that various Cannabis tissues have been successfully transiently transformed using biolistics system. Transformation has been performed into calli, leaves, axillary buds and cotyledons of Cannabis.

According to further embodiments of the present invention, additional transformation tools were used in Cannabis, including, but not limited to:
Protoplast PEG transformation
Extend RNP use
Directed editing screening using fluorescent tags
Electroporation

Stage 4: Regeneration in tissue-culture. When transforming DNA constructs into the plant, antibiotics is used for selection of positive transformed plants. An improved regeneration protocol was herein established for the Cannabis plant.

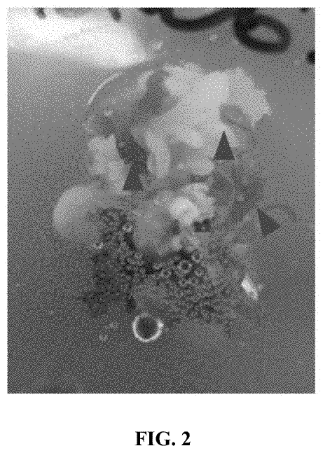

Reference is now made to FIG. 2 presenting regeneration of Cannabis tissue. In this figure, arrows indicate new meristem emergence.

Stage 5: Selection of positive transformants. Once regenerated plants appear in tissue culture, DNA is extracted from leaf sample of the transformed plant and PCR is performed using primers flanking the edited region. PCR products are then digested with enzymes recognizing the restriction site near the original gRNA sequence. If editing event occurred, the restriction site will be disrupted and the PCR product will not be cleaved. No editing event will result in a cleaved PCR product.

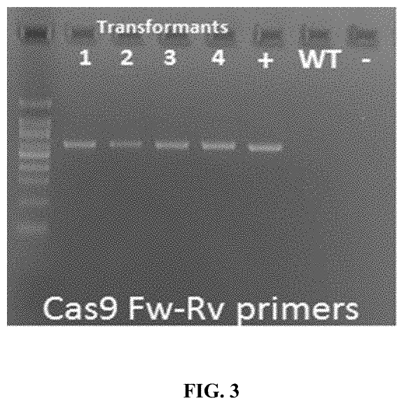

Reference is now made to FIG. 3 showing PCR detection of Cas9 DNA in shoots of transformed Cannabis plants. DNA extracted from shoots of plants transformed with Cas9 using biolistics. This figure shows that three weeks post transformation, Cas9 DNA was detected in shoots of transformed plants.

Screening for CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)

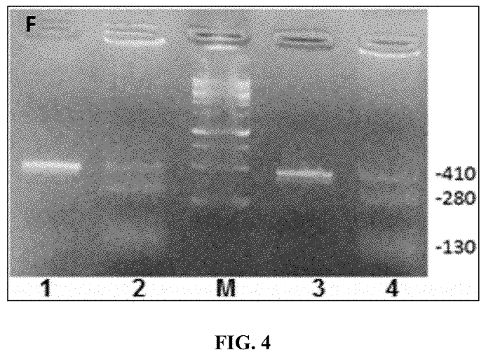

Reference is now made to FIG. 4 presenting results of in vitro analysis of CRISPR/Cas9 cleavage activity. The genomic area targeted for editing was amplified by the reverse and forward designed primers. FIG. 4 photographically presents a gel showing successful digestion of the resulted PCR amplicon containing the gene specific gRNA sequence, by RNP complex containing Cas9. The analysis included the following steps:
1) Amplicon was isolated from two exemplified Cannabis strains by primers flanking the sequence of the gene of interest targeted by the predesigned sgRNA.
2) RNP complex was incubated with the isolated amplicon.
3) The reaction mix was then loaded on agarose gel to evaluate Cas9 cleavage activity at the target site.

Stage 6: Selection of transformed Cannabis plants presenting sp and sp5g related phenotypes as described above. It is within the scope that different gRNA promoters were tested in order to maximize editing efficiency.

### EXAMPLE 2

Production of mutated Cssp-1 gene alleles by genome editing events

This example presents the production of new editing events within CsSP-1 gene.

Three single guide RNAs (sgRNA) targeting selected regions within the genomic sequence of CsSP-1 gene were designed and synthesized. These gRNAs include gRNA having nucleotide sequence as set forth in SEQ ID NO: 102 (first guide), SEQ ID NO: 109 (second guide) and SEQ ID NO: 112 (third guide), starting at position 3291, 3260 and 3167 of SEQ ID NO: 1 (WT CsSP-1 genomic sequence), respectively. The predicted Cas9 cleavage sites directed by these guide RNAs were designed to overlap with the nucleic acid recognition site of the restriction enzymes BsaXI, XapI and RseI for the first, second and third guide, respectively (see Tables 9-12). Transformation was performed using a DNA plasmid such as a plant codon optimized Streptococcus pyogenes Cas9 (pcoSpCas9) plasmid. The plasmid contained the plant codon optimized SpCas9 and the above mentioned at least one gRNA. Leaves from mature transformed plants were sampled, and their DNA was extracted and digested with the suitable enzymes. Digested genomic DNA was used as a template for PCR using a primer pair flanking the 5′ and 3′ ends of the predicted cleavage site of CsSP-1.

Tables 9-12 present the sequence of the resultant mutated Cssp-1 fragments containing gene-editing events, as compared to the corresponding WT non edited CsSP-1 fragment sequence. In these tables, gRNA sequences are underlined; PAM sequences (NGG) are presented in bold.

Reference is now made to Table 9 presenting nucleic acid sequence comparison of mutated Cssp-1 fragments containing genome editing events obtained using the first guide having nucleic acid sequence as set forth in SEQ ID NO: 102, and the corresponding WT CsSP-1 sequence.

TABLE 9
Cssp-1 gene editing events obtained using gRNA having a nucleic acid sequence
as set forth in SEQ ID NO: 102

| | | Indel | |
|---|---|---|---|
| | | posi- | |
| | | tion | |
| | | in | |
| | | SEQ | SEQ |
| +/− | | ID | ID: |
| bp | sequence | NO: 1 | NO |
| WT | 2373 TGTCAGATAATCCTCTTATTATTGGAAGAGTGATAGGAG-ATGTGGTTGATGTTTTCTCTCCAAC 2310 | | 917 |
| −1 | 2372 TGTCAGATAATCCTCTTATTATTGGAAGAGTGATAGGA--ATGTGGTTGATGTTTTCTCTCCAAC 2310 | 2335 | 918 |
| −2 | 2371 TGTCAGATAATCCTCTTATTATTGGAAGAGTGATAG--G-ATGTGGTTGATGTTTTCTCTCCAAC 2310 | 2336 | 919 |
| +1 | 2374 TGTCAGATAATCCTCTTATTATTGGAAGAGTGATAGGAGAATGTGGTTGATGTTTTCTCTCCAAC 2310 | 2335 | 920 |
| −5 | 2368 TGTCAGATAATCCTCTTATTATTGGAAGAGTGA-------ATGTGGTTGATGTTTTCTCTCCAAC 2310 | 2334 | 921 |
| −4 | 2369 TGTCAGATAATCCTCTTATTATTGGAAGAGTGATA-----ATGTGGTTGATGTTTTCTCTCCAAC 2310 | 2334 | 922 |

Reference is now made to Table 10 presenting nucleic acid sequence comparison of mutated Cssp-1 fragments containing genome editing events obtained using the first guide having nucleic acid sequence as set forth in SEQ ID NO: 109, and the corresponding WT CsSP-1 sequence.

TABLE 10
Cssp-1 gene editing events obtained using gRNA having a nucleic acid
sequence as set forth in SEQ ID NO: 109

| | | Indel | |
|---|---|---|---|
| | | Posi- | |
| | | tion | SEQ |
| +/− | | SEQ ID | ID: |
| bp | sequence | NO: 1 | NO |
| WT | 2328 TTGATGTTTTCTCTCCAACCCTAAAATTTACAGTGACTTACAACTCAAACAAGAAAGTCTACAA 2265 | | 923 |
| −1 | 2327 TTGATGTTTTCTCTCCAACCCTAAA-TTTACAGTGACTTACAACTCAAACAAGAGAGTCTACAA 2265 | 2302 | 924 |
| −46 | 2282 TTGATGTTTTCTCT----------------------------------------------ACAA 2265 | 2269 | 925 |

Reference is now made to Table 11 presenting nucleic acid sequence comparison of mutated Cssp-1 fragments containing genome editing events obtained using the first guide having nucleic acid sequence as set forth in SEQ ID NO: 112, and the corresponding WT CsSP-1 sequence.

TABLE 11
Cssp-1 gene editing events obtained using gRNA having a nucleic acid sequence as
set forth in SEQ ID NO: 112

| | | Indel | |
|---|---|---|---|
| | | posi- | |
| | | tion | |
| | | in | |
| | | SEQ | SEQ |
| +/− | | ID | ID: |
| bp | sequence | NO: l | NO |
| WT | 2249 TCCTTCCACAATTACTATTAAGCCAAAAGTTGAGGTTCA-TGGTGGTGACATGAGATCCTTTTTC 2186 | | 926 |
| +1 | 2290 TCCTTCCACAATTACTATTAAGCCAAAAGTTGAGGTTCAATGGTGGTGACATGAGATCCTTTTTC 2186 | 2211 | 927 |
| −1 | 2248 TCCTTCCACAATTACTATCAAGCCAAAAGTTGAGGTTCA--GGTGGTGACATGAGATCCTTTTTC 2186 | 2210 | 928 |
| −6 | 2243 TCCTTCCACAATTACTATTAAGCCAAAAGTTGA-------TGGTGGTGACATGAGATCCTTTTTC 2186 | 2211 | 929 |
| −2 | 2247 TCCTTCCACAATTACTATTAAGCCAAAAGTTGAGGTTC---GGTGGTGACATGAGATCCTTTTTC 2186 | 2210 | 930 |
| −3 | 2246 TCCTTCCACAATTACTATTAAGCCAAAAGTTGAGGT----TGGTGGTGACATGAGATCCTTTTTC 2186 | 2211 | 931 |
| −2 | 2247 TCCTTCCACAATTACTATTAAGCCAAAAGTTGAGGTT---TGGTGGTGACATGAGATCCTTTTTC 2186 | 2211 | 932 |
| −1 | 2248 TCCTTCCACAATTACTATTAAGCCAAAAGTTGAGGTTC--TGGTGGTGACATGAGATCCTTTTTC 2186 | 2211 | 933 |

Reference is now made to Table 12 presenting nucleic acid sequence comparison of mutated Cssp-1 fragments containing genome editing events obtained using the combination of the first, second and third guides having nucleic acid sequence as set forth in SEQ ID NO: 102, SEQ ID NO: 109 and SEQ ID NO: 112, respectively, and the corresponding WT CsSP-1 sequence.

TABLE 12
Cssp-1 gene editing events obtained using the combination of gRNAs having a
nucleic acid sequence as set forth in SEQ ID NO: 102, SEQ ID NO: 109
and SEQ ID NO: 112

| | | Indel | |
|---|---|---|---|
| | | posi- | |
| | | tion | |
| | | in | |
| | | SEQ | |
| +/− | | ID | SEQ |
| bp | sequence | NO: 1 | ID: |
| WT | 2365 AATCCTCTTATTATTGGAAGAGTGATAGGAG- | | 934 |
| | ATGTGGTTGATGTTTTCTCTCCAACCCTAAAATTTACAGTGACTTACAACTCAAACAAGAGAGTCTACAATGGCCA | | |
| | TGAGTTGTTTCCTTCCACAATTACTATTAAGCCAAAAGTTGAGGTTCATGGTGGTGACATGAGATCC 2191 | | |
| −124 | 2241 AATCCTCTTATTATTGGAAGAGTGATAGGAG- | 2211 | 935 |
| | ---------------------------------------------------------------------------- | | |
| | ------------------------------------------------TGGTGGTGACATGAGATCC 2191 | | |
| −123 | 2242 AATCCTCTTATTATTGGAAGAGTGATAGGAG- | 2211 | 936 |
| | A--------------------------------------------------------------------------- | | |
| | ------------------------------------------------TGGTGGTGACATGAGATCC 2191 | | |
| −31 | 2334 AATCCTCTTATTATTGGAAGAGTGATAGGAG- | 2304 | 937 |
| | -------------------------------ATTTACAGTGACTTACAACTCAAACAAGAGAGTCTACAATGGCCA | | |
| | TGAGTTGTTTCCTTCCACAATTACTATTAAGCCAAAAGTTGAGGTTCATGGTGGTGACATGAGATCC 2191 | | |
| −91 | 2274 AATCCTCTTATTATTGGAAGAGTGATAGGAGA | 2211 | 938 |
| | ATGTGGTTGATGTTTTCTCTCCAACCCTAAAA-------------------------------------------- | | |
| | ------------------------------------------------TGGTGGTGACATGAGATCC 2191 | | |

Reference is now made to Table 13 presenting summary of WT CsSP-1 fragments and mutated Cssp-1 fragments containing gene editing events, within the scope of the present invention. In this table, “d” represents deletion and “i” represents “insertion”, followed by the number of base pairs (bp) inserted or deleted.

TABLE 13
Summary of WT CsSP-1 and mutated Cssp-1-related
sequences within the scope of the present invention

| | | Indel | Insersion | |
|---|---|---|---|---|
| | SEQ | position in | (+)/ | Targeted |
| | ID: | SEQ ID | deletion | gRNA SEQ |
| Sequence name | NO | NO: 1 | (−) bp | ID: NO |
| CsSP-1_2373-2310 | 917 | | | |
| Cssp-1_2335_d1 | 918 | 2335 | −1 | 102 |
| Cssp-1_2336_d2 | 919 | 2336 | −2 | 102 |
| Cssp-1_2335_i1 | 920 | 2335 | +1 | 102 |
| Cssp-1_2334_d5 | 921 | 2334 | −5 | 102 |
| Cssp-1_2334_d4 | 922 | 2334 | −4 | 102 |
| CsSP-1_2328-2265 | 923 | | | |
| Cssp-1_2302_d1 | 924 | 2302 | −1 | 109 |
| Cssp-1_2269_d46 | 925 | 2269 | −46 | 109 |
| CsSP-1_2249-2186 | 926 | | | |
| Cssp-1_2211_i1 | 927 | 2211 | +1 | 112 |
| Cssp-1_2210_d1 | 928 | 2210 | −1 | 112 |
| Cssp-1_2211_d6 | 929 | 2211 | −6 | 112 |
| Cssp-1_2210_d2 | 930 | 2210 | −2 | 112 |
| Cssp-1_2211_d3 | 931 | 2211 | −3 | 112 |
| Cssp-1_2211_d2 | 932 | 2211 | −2 | 112 |
| Cssp-1_2211_d1 | 933 | 2211 | −1 | 112 |
| CsSP-1_2365-2191 | 934 | | | |
| Cssp-1_2211_d124 | 935 | 2211 | −124 | 102 + 109 + 112 |
| Cssp-1_2211_d123 | 936 | 2211 | −123 | 102 + 109 + 112 |
| Cssp-1_2304_d31 | 937 | 2304 | −31 | 102 + 109 + 112 |
| Cssp-1_2211_d91 | 938 | 2211 | −91 | 102 + 109 + 112 |

The tables above demonstrate that Cannabis plants with mutated Cssp-1 alleles containing the above identified DNA fragment sequences were achieved, by the gene editing method of the present invention. Each of the alleles encompass at least one insertion or deletion gene- editing event within the CsSP-1 gene, targeted by one or more predesigned gRNA molecules. The generated mutated Cssp-1 gene alleles are expected to result is a non-functional, silenced Cssp-1 gene, conferring improved agronomic or domestication trait in Cannabis.

According to further aspects of the present invention, the sequences of the mutated Cssp-1 DNA fragments provided by the present invention, as well as any functional variant or partial sequence thereof, is useful to identify and generate Cannabis plants with mutated CsSP-1 gene alleles, desirable for the production of Cannabis plants with improved agronomic or domestication trait.

The genome editing events herein described introduce mutations that silence or significantly reduce CsSP-1 gene expression or function in the plant.

By silencing the gene encoding SP1 protein in Cannabis, plants with improved agronomic trait such as ‘determinate’ growth habit, are produced. These sp1-knockout Cannabis plants are highly desirable since their development can be regulated. More specifically, the regularity of the vegetative-reproductive switch is controlled to produce a high yield phenotype, for example due to allowing higher planting density and synchronized growth.

### REFERENCES

Sebastian Soyk, Niels A Müller, Soon Ju Park, Inga Schmalenbach, Ke Jiang, Ryosuke Hayama, Lei Zhang, Joyce Van Eck, José M Jiménez-Gómez & Zachary B Lippman “Variation in the flowering gene SELF PRUNING 5G promotes day-neutrality and early yield in tomato” Nature Genetics, 2017 49, 162-168.

Tingdong Li, Xinping Yang, Yuan Yu, Xiaomin Si, Xiawan Zhai, Huawei Zhang, Wenxia Dong, Caixia Gao and Cao Xu “Domestication of wild tomato is accelerated by genome editing” Nature Biotechnology, 2018 36, 1160-1163.

Agustin Zsögön, Tomáš Čermák, Emmanuel Rezende Naves, Marcela Morato Notini, Kai H Edel, Stefan Weinl, Luciano Freschi, Daniel F Voytas, Jörg Kudla and Lázar° Eustáquio Pereira Peres “De novo domestication of wild tomato using genome editing” Nature Biotechnology, 2018 36, 1211-1216.

Zachary H. Lemmon, Nathan T. Reem, Justin Dalrymple, Sebastian Soyk, Kerry E. Swartwood, Daniel Rodriguez-Leal, Joyce Van Eck and Zachary B. Lippman “Rapid improvement of domestication traits in an orphan crop by genome editing” Nature Plants, 2018 4, 766-770.

Xie, K. and Yang Y. “RNA-guided genome editing in plants using a CRISPR—Cas system.” Molecular plant, 2013 6 (6), 1975-1983.

1. A modified Cannabis plant exhibiting at least one improved domestication trait compared with wild type Cannabis, wherein said modified plant comprises at least one mutated Cannabis SELF PRUNING (SP) (CsSP) gene selected from the group consisting of CsSP-1 having a genomic nucleotide sequence as set forth in SEQ ID NO: 1 or a functional variant thereof, CsSP-2 having a genomic nucleotide sequence as set forth in SEQ ID NO: 4 or a functional variant thereof, CsSP-3 having a genomic nucleotide sequence as set forth in SEQ ID NO: 7 or a functional variant thereof and any combination thereof, and/or at least one mutated Cannabis SELF PRUNING 5G (SP5G) (CsSP5G) gene selected from the group consisting of CsSP5G-1 having a genomic nucleotide sequence as set forth in SEQ ID NO: 10 or a functional variant thereof, CsSP5G-2 having a genomic nucleotide sequence as set forth in SEQ ID NO: 13 or a functional variant thereof, CsSP5G-3 having a genomic nucleotide sequence as set forth in SEQ ID NO: 16 or a functional variant thereof, CsSP5G-4 having a genomic nucleotide sequence as set forth in SEQ ID NO: 19 or a functional variant thereof and any combination thereof.

2. The modified Cannabis plant according to claim 1, wherein said functional variant has at least 75% sequence identity to said CsSP or said CsSP5G nucleotide sequence.

3. The modified Cannabis plant according to claim 1, wherein said mutation is introduced using mutagenesis, small interfering RNA (siRNA), microRNA (miRNA), artificial miRNA (amiRNA), DNA introgression, endonucleases or any combination thereof

4. The modified Cannabis plant according to claim 1, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof

5. The modified Cannabis plant according to claim 1, wherein the mutated CsSP or CsSP5G gene is a CRISPR/Cas9- induced heritable mutated allele.

6. The modified Cannabis plant of claim 1, wherein said plant is homozygous for said at list one CsSP or said at list one CsSP5G mutated gene, or said plant is a Cssp Cssp5g double mutant.

7. The modified Cannabis plant according to claim 1, wherein at least one of the following holds true:
a. said mutation in said CsSP-1 is generated in planta via introduction of a construct comprising (i) Cas DNA and gRNA sequence selected from the group consisting of SEQ ID NO: 22-SEQ ID NO: 126 and any combination thereof, or (ii) a ribonucleoprotein (RNP) complex comprising Cas protein and gRNA sequence selected from the group consisting of SEQ ID NO: 22-126 and any combination thereof;
b. said mutation in said CsSP-2 is generated in planta via introduction of a construct comprising (i) Cas DNA and gRNA sequence selected from the group consisting of SEQ ID NO: 127-SEQ ID NO: 211 and any combination thereof, or (ii) a ribonucleoprotein (RNP) complex comprising Cas protein and gRNA sequence selected from the group consisting of SEQ ID NO: 127-211 and any combination thereof;
c. said mutation in said CsSP-3 is generated in planta via introduction of a construct comprising (i) Cas DNA and gRNA sequence selected from the group consisting of SEQ ID NO: 212-SEQ ID NO: 283 and any combination thereof, or (ii) a ribonucleoprotein (RNP) complex comprising Cas protein and gRNA sequence selected from the group consisting of SEQ ID NO: 212-283 and any combination thereof;
d. said mutation in said CsSP5G-1 is generated in planta via introduction of a construct comprising (i) Cas DNA and gRNA sequence selected from the group consisting of SEQ ID NO: 284-SEQ ID NO: 516 and any combination thereof, or (ii) a ribonucleoprotein (RNP) complex comprising Cas protein and gRNA sequence selected from the group consisting of SEQ ID NO: 284-516 and any combination thereof;
e. said mutation in said CsSP5G-2 is generated in planta via introduction of a construct comprising (i) Cas DNA and gRNA sequence selected from the group consisting of SEQ ID NO: 517-SEQ ID NO: 745 and any combination thereof, or (ii) a ribonucleoprotein (RNP) complex comprising Cas protein and gRNA sequence selected from the group consisting of SEQ ID NO: 517-745 and any combination thereof;
f. said mutation in said CsSP5G-3 is generated in planta via introduction of a construct comprising (i) Cas DNA and gRNA sequence selected from the group consisting of SEQ ID NO: 746-SEQ ID NO: 828 and any combination thereof, or (ii) a ribonucleoprotein (RNP) complex comprising Cas protein and gRNA sequence selected from the group consisting of SEQ ID NO: 746-828 and any combination thereof; and
g. said mutation in said CsSP5G-4 is generated in planta via introduction of a construct comprising (i) Cas DNA and gRNA sequence selected from the group consisting of SEQ ID NO: 829-SEQ ID NO: 916 and any combination thereof, or (ii) a ribonucleoprotein (RNP) complex comprising Cas protein and gRNA sequence selected from the group consisting of SEQ ID NO: 829-916 and any combination thereof.

8. The modified Cannabis plant according to claim 1, wherein said plant has decreased expression levels of at least one of said CsSP genes, and/or decreased expression levels of at least one of said CsSP5G genes.

9. The modified Cannabis plant according to claim 1, wherein said domestication trait is selected from the group consisting of reduced flowering time, earliness, synchronous flowering, earlier flowering, suppressed or reduced day-length sensitivity, determinant or semi-determinant architecture or growth habit, early termination of sympodial cycling, suppressed sympodial shoot termination, similar sympodial shoot termination as compared to a corresponding wild type cannabis plant, earlier axillary shoot flowering, compact growth habit, reduced height, reduced number of sympodial units, adaptation to mechanical harvest, higher harvest index and any combination thereof

10. The modified Cannabis plant according to claim 6, wherein said Cssp Cssp5g double mutant is characterized by having a more than additive effect on a trait selected from the group consisting of compactness, earlier axillary shoot flowering, earlier termination of sympodial cycling, harvest index and any combination thereof as compared to wild type and/or sp mutant Cannabis plants.

11. The modified Cannabis plant according to claim 1, wherein said modified plant comprises a mutated Cannabis self pruning (sp)-1 (Cssp-1) gene allele, said mutated allele comprising a genomic modification selected from an indel at position corresponding to position 2210, 2211, 2269, 2302, 2304 2334, 2335, 2336, and any combination thereof, of Cannabis SP-1 (CsSP-1) gene having a polynucleotide sequence corresponding to a sequence having at least 80% sequence identity to the sequence as set forth in SEQ ID NO: 1, or a functional fragment or variant thereof.

12. The modified Cannabis plant according to claims 11, wherein said Cssp-1 allele comprises an indel at a polynucleotide sequence corresponding to a sequence having at least 80% sequence identity to the sequence as set forth in SEQ ID NO: 1 selected from 1 bp deletion at position 2335, 2 bp deletion at position 2336, 1 bp insertion at position 2335, 5 bp deletion at position 2334, 4 bp deletion at position 2334, 1 bp deletion at position 2302, 46 bp deletion at position 2269, 1 bp insertion at position 2211, 1 bp deletion at position 2210, 6 bp deletion at position 2211, 2 bp deletion at position 2210, 3 bp deletion at position 2211, 2 bp deletion at position 2211, 1 bp deletion at position 2211, 124 bp deletion at position 2211, 123 bp deletion at position 2211, 31 bp deletion at position 2304, 91 bp deletion at position 2211 and any combination thereof.

13. The modified Cannabis plant according to claim 11, wherein said Cssp-1 allele comprises a polynucleotide sequence having at least 80% identity to a polynucleotide sequence selected from SEQ ID NO: 918-922, SEQ ID NO: 924-925, SEQ ID NO: 927-933, SEQ ID NO: 935-938, or a complementary sequence thereof, or any combination thereof.

14. The modified Cannabis plant according to claim 11, wherein said mutated Cssp-1 allele confers improved domestication trait as compared to a Cannabis plant comprising a wild type CsSP-1 allele comprising a polynucleotide sequence having at least 80% identity to a polynucleotide sequence selected from SEQ ID NO: 917, SEQ ID NO: 923, SEQ ID NO:
926, SEQ ID NO: 934 or any combination thereof, and/or having a polynucleotide sequence having at least 80% identity to the polynucleotide sequence as set forth in SEQ ID NO: 1.

15. The modified Cannabis plant according to claim 11, wherein said modified plant is generated via introduction of a gRNA comprising a polynucleotide sequence corresponding to a sequence selected from the group consisting of SEQ ID NO: 102, SEQ ID NO: 109 and SEQ ID NO: 112, a complementary sequence thereof, and any combination thereof.

16. A plant part, plant cell, tissue culture of regenerable cells, protoplasts or callus or plant seed of a plant according to claim 1.

17. A method for producing a modified Cannabis plant according to claim 1, said method comprises steps of genetically introducing by targeted genome modification, a loss of function mutation in at least one Cannabis SELF PRUNING (SP) (CsSP) gene selected from the group consisting of CsSP-1 having a genomic nucleotide sequence as set forth in SEQ ID NO: 1 or a functional variant thereof, CsSP-2 having a genomic nucleotide sequence as set forth in SEQ ID NO: 4 or a functional variant thereof, CsSP-3 having a genomic nucleotide sequence as set forth in SEQ ID NO: 7 or a functional variant thereof and any combination thereof, and/or at least one Cannabis SELF PRUNING 5G (SPSG) (CsSP5G) gene selected from the group consisting of CsSP5G-1 having a genomic nucleotide sequence as set forth in SEQ ID NO: 10 or a functional variant thereof, CsSP5G-2 having a genomic nucleotide sequence as set forth in SEQ ID NO: 13 or a functional variant thereof, CsSP5G-3 having a genomic nucleotide sequence as set forth in SEQ ID NO: 16 or a functional variant thereof, CsSP5G-4 having a genomic nucleotide sequence as set forth in SEQ ID NO: 19 or a functional variant thereof and any combination thereof.

18. A method of improving at least one domestication trait compared with wild type Cannabis, comprising steps of producing a modified Cannabis plant according to claim 1, seed or plant part thereof, that is homozygous for at least one mutated CsSP5G gene selected from the group consisting of CsSP5G-1 having a genomic nucleotide sequence as set forth in SEQ ID NO: 10 or a functional variant thereof, CsSP5G-2 having a genomic nucleotide sequence as set forth in SEQ ID NO: 13 or a functional variant thereof, CsSP5G-3 having a genomic nucleotide sequence as set forth in SEQ ID NO: 16 or a functional variant thereof, CsSP5G-4 having a genomic nucleotide sequence as set forth in SEQ ID NO: 19 or a functional variant thereof and any combination thereof in a sp background and enabling growth of said Cannabis plant, seed or plant part thereof.

19. The method according to claim17, wherein said method comprises steps of:
a. identifying at least one Cannabis SP (CsSP) and/or at least one Cannabis SP5G (CsSP5G) allele;
b. synthetizing at least one guide RNA (gRNA) comprising a nucleotide sequence complementary to said at least one identified CsSP and/or CsSP5G allele;
c. transforming Cannabis plant cells with a construct comprising (a) Cas nucleotide sequence operably linked to said at least one gRNA, or (b) a ribonucleoprotein (RNP) complex comprising Cas protein and said at least one gRNA;
d. screening the genome of said transformed plant cells for induced targeted loss of function mutation in at least one of said CsSP and/or CsSP5G allele;
e. regenerating Cannabis plants carrying said loss of function mutation in at least one of said CsSP and/or CsSP5G allele; and
f. screening said regenerated plants for a Cannabis plant with improved domestication trait.

20. The method according to claim 19, wherein at least one of the following holds true:
a. said mutation in said CsSP-1 is generated in planta via introduction of a construct comprising (i) Cas DNA and gRNA sequence selected from the group consisting of SEQ ID NO: 22-SEQ ID NO: 126 and any combination thereof, or (ii) a ribonucleoprotein (RNP) complex comprising Cas protein and gRNA sequence selected from the group consisting of SEQ ID NO: 22-126 and any combination thereof;
b. said mutation in said CsSP-2 is generated in planta via introduction of a construct comprising (i) Cas DNA and gRNA sequence selected from the group consisting of SEQ ID NO: 127-SEQ ID NO: 211 and any combination thereof, or (ii) a ribonucleoprotein (RNP) complex comprising Cas protein and gRNA sequence selected from the group consisting of SEQ ID NO: 127-211 and any combination thereof;
c. said mutation in said CsSP-3 is generated in planta via introduction of a construct comprising (i) Cas DNA and gRNA sequence selected from the group consisting of SEQ ID NO: 212-SEQ ID NO: 283 and any combination thereof, or (ii) a ribonucleoprotein (RNP) complex comprising Cas protein and gRNA sequence selected from the group consisting of SEQ ID NO: 212-283 and any combination thereof;
d. said mutation in said CsSP5G-1 is generated in planta via introduction of a construct comprising (i) Cas DNA and gRNA sequence selected from the group consisting of SEQ ID NO: 284-SEQ ID NO: 516 and any combination thereof, or (ii) a ribonucleoprotein (RNP) complex comprising Cas protein and gRNA sequence selected from the group consisting of SEQ ID NO: 284-516 and any combination thereof;
e. said mutation in said CsSP5G-2 is generated in planta via introduction of a construct comprising (i) Cas DNA and gRNA sequence selected from the group consisting of SEQ ID NO: 517-SEQ ID NO: 745 and any combination thereof, or (ii) a ribonucleoprotein (RNP) complex comprising Cas protein and gRNA sequence selected from the group consisting of SEQ ID NO: 517-745 and any combination thereof;
f. said mutation in said CsSP5G-3 is generated in planta via introduction of a construct comprising (i) Cas DNA and gRNA sequence selected from the group consisting of SEQ ID NO: 746-SEQ ID NO: 828 and any combination thereof, or (ii) a ribonucleoprotein (RNP) complex comprising Cas protein and gRNA sequence selected from the group consisting of SEQ ID NO: 746-828 and any combination thereof; and
g. said mutation in said CsSP5G-4 is generated in planta via introduction of a construct comprising (i) Cas DNA and gRNA sequence selected from the group consisting of SEQ ID NO: 829-SEQ ID NO: 916 and any combination thereof, or (ii) a ribonucleoprotein (RNP) complex comprising Cas protein and gRNA sequence selected from the group consisting of SEQ ID NO: 829-916 and any combination thereof.

21. A Cannabis plant, plant part, plant seed, tissue culture of regenerable cells, protoplasts, callus or plant cell produced by the method according to claim 17.

22. An isolated polynucleotide sequence having at least 75% sequence identity to a polynucleotide sequence selected from the group consisting of SEQ ID NO: 1-2, SEQ ID NO: 4-5, SEQ ID NO: 7-8, SEQ ID NO: 10-11, SEQ ID NO: 13-14, SEQ ID NO: 16-17, SEQ ID NO: 19-20, SEQ ID NO: 22-283, SEQ ID NO: 284-916, SEQ ID NO: 918-922, SEQ ID NO: 924-925, SEQ ID NO: 927-933 and SEQ ID NO: 935-938, or an isolated amino acid sequence having at least 75% sequence similarity to amino acid sequence selected from the group consisting of SEQ ID NO: 3, SEQ ID NO: 6,SEQ ID NO: 9, SEQ ID NO: 12, SEQ ID NO: 15, SEQ ID NO: 18 and SEQ ID NO: 21.

23. A method for generating, identifying and/or screening for a Cannabis plant according to claim 11, comprising detecting the presence of at least one polynucleotide sequence selected from the group consisting of a sequence having at least 80% identity to SEQ ID NO: 918-922, SEQ ID NO: 924-925, SEQ ID NO: 927-933, SEQ ID NO: 935-938, or a complementary sequence thereof, and a combination thereof.

24. A detection kit for identifying a Cannabis plant with improved domestication trait by determining the presence or absence of a mutant Cssp-1 allele in a Cannabis plant, comprising a polynucleotide fragment having at least 80% identity to SEQ ID NO: 918-922, SEQ ID NO: 924-925, SEQ ID NO: 927-933, SEQ ID NO: 935-938, or a complementary sequence thereof, and any combination thereof.
